SEQUENCE LISTING (80 sequences)

SEQ ID NO: 1 - DNA, 9 bp, Artificial Sequence
  Description of Artificial Sequence; note = synthetic construct
nyrggagar

SEQ ID NO: 2 - RNA, 134 nt, Artificial Sequence
  Description of Artificial Sequence; note = synthetic construct
ggguugaagacugcaggagagugguuguuaaccagauuuuaacaucugagccaaauaacccgccgaagaaguaaaucuuuacggugcauuauucuuagccauauauuggcaacgaauaagcgaggacuguaguu

SEQ ID NO: 3 - RNA, 60 nt, Artificial Sequence
  Description of Artificial Sequence; note = synthetic construct
ccucuggagagaaccguuuaaucggucgccgaaggagcaagcucugcgcauaugcagagu

SEQ ID NO: 4 - RNA, 105 nt, Artificial Sequence
  Description of Artificial Sequence; note = synthetic construct
ggacuguaguuggaggaaccucuggagagaaccguuuaaucggucgccgaaggagcaagcucugcgcauaugcagagugaaacucucaggcaaaaggacagagga

SEQ ID NO: 5 - RNA, 131 nt, Artificial Sequence
  Description of Artificial Sequence; note = synthetic construct
ggguugaagacugcaggagagugguuguuaaccagauuuuaacaucugagccaaauaacccgccgaaguaaaucuuuacggugcauuauucuuagccauauauuggcaacgaauaagcgaggacuguaguu

SEQ ID NO: 6 - RNA, 83 nt, Artificial Sequence
  Description of Artificial Sequence; note = synthetic construct
yrggagarcrccgaagrgyaaacyyucaggyrrracyryrrucuggaragcrccgaagggaraacucucaggyrrrgagagrr

SEQ ID NO: 7 - RNA, 238 nt, Artificial Sequence
  Description of Artificial Sequence; note = synthetic construct
uaauucggaugaaccauucaggagaaggucuauugaucuaccgacggggcaaaaaguuguuaaccgacuuugaaacucucaggucuuguuuacaaguagaacugcauggggacgaaucucuggagagacucccucucgcuuuaaauagcguagaggaaaacgagcaccgaaggagcaaauccgcuacuauagcggauaaucucucagguaaaaggacagagacaagcgaaagaaaaug

SEQ ID NO: 8 - DNA, 41 bp, Artificial Sequence
  Description of Artificial Sequence; note = synthetic construct
taatacgactcactatagggttgaagactgcaggagagtgg

SEQ ID NO: 9 - DNA, 20 bp, Artificial Sequence
  Description of Artificial Sequence; note = synthetic construct
tcctctgtccttttgcctga

SEQ ID NO: 10 - DNA, 43 bp, Artificial Sequence
  Description of Artificial Sequence; note = synthetic construct
taatacgactcactatagggatatgagcgaatgacagcaaggg

SEQ ID NO: 11 - DNA, 35 bp, Artificial Sequence
  Description of Artificial Sequence; note = synthetic construct
ggttctctgtcctggcacctgaaagtttactttgc

SEQ ID NO: 12 - DNA, 67 bp, Artificial Sequence
  Description of Artificial Sequence; note = synthetic construct
cagcctatgcaagagattagaatcttgatataatttattacaagatgaataatataagaaaaatctg

SEQ ID NO: 13 - DNA, 14 bp, Artificial Sequence
  Description of Artificial Sequence; note = synthetic construct
crccgaagrngyaa

SEQ ID NO: 14 - RNA, 11 nt, Artificial Sequence
  Description of Artificial Sequence; note = synthetic construct
ancyyucaggy

SEQ ID NO: 15 - DNA, 8 bp, Artificial Sequence
  Description of Artificial Sequence; note = synthetic construct
rrracyry

SEQ ID NO: 16 - RNA, 10 nt, Artificial Sequence
  Description of Artificial Sequence; note = synthetic construct
nucuggarag

SEQ ID NO: 17 - DNA, 14 bp, Artificial Sequence
  Description of Artificial Sequence; note = synthetic construct
crccgaaggngnar

SEQ ID NO: 18 - RNA, 12 nt, Artificial Sequence
  Description of Artificial Sequence; note = synthetic construct
aancucucaggy

SEQ ID NO: 19 - DNA, 10 bp, Artificial Sequence
  Description of Artificial Sequence; note = synthetic construct
rrrgacagag

SEQ ID NO: 20 - DNA, 7 bp, Artificial Sequence
  Description of Artificial Sequence; note = synthetic construct
ggaggaa

SEQ ID NO: 21 - RNA, 87 nt, Artificial Sequence
  Description of Artificial Sequence; note = synthetic construct
ccucuggagagaaccguuuaaucggucgccgaaggagcaagcucugcgcauaugcagagugaaacucugaggcaaaaggacagagga

SEQ ID NO: 22 - RNA, 111 nt, Artificial Sequence
  Description of Artificial Sequence; note = synthetic construct
gggauaugagcgaaugacagcaaggggagagaccugaccgaaaaccucgggauacaggcgccgaaggagcaaacugcggagugaaucucucaggcaaaagaacucuugcuc

SEQ ID NO: 23 - DNA, 7 bp, Artificial Sequence
  Description of Artificial Sequence; note = synthetic construct
gacgcaa

SEQ ID NO: 24 - RNA, 125 nt, Artificial Sequence
  Description of Artificial Sequence; note = synthetic construct
cucuggagaguguuugugcggaugcgcaaaccaccaaaggggacguuuugcguaugcaaaguaaacuuucaggugccaggacagagaaccuucauuuuacaugagguguuucucuguccuuuuuu

SEQ ID NO: 25 - RNA, 240 nt, Artificial Sequence
  Description of Artificial Sequence; note = synthetic construct
uaauucggaugaaccauucaggagaaggucuauugaucuaccgacggggcaaaaaguuguuauaccagcuuugaaacucucaggucuugguuacaaguagaacugcauggggacgaaucucuggagagacucccucucgcuuucauagagcgcggaggaaaacgagcaccgaaggagcaaauccgcuacuuuagcggauaaucucucagguaaaaggacagagacaagcgaaagaaaaag

SEQ ID NO: 26 - RNA, 215 nt, Artificial Sequence
  Description of Artificial Sequence; note = synthetic construct
aaaagcggaugaaagcaaggggagagacugcaaggaugcagcgccgaaggagcaaacacaauuagggugugaaucucucaggcaaaaagacucuugcucgacgcaggcagcucuggagagcgucuaacacuagaccaccuacgaagacauuuccuuuuuacgauaaggggaaagaaacuuucugguaaccggacagagcuuuacacaacuuacug

SEQ ID NO: 27 - RNA, 217 nt, Artificial Sequence
  Description of Artificial Sequence; note = synthetic construct
augagcgaaugacagcaaggggagagaccugaccgaaaaccucgggauacaggcgccgaaggagcaaacugcggagugaaucucucaggcaaaagaacucuugcucgacgcaacucuggagaguguuugugcggaugcgcaaaccaccuuuggggacgucuuugcguaugcaaaguaaacuuucaggugccaggacagagaaccuucauuuuacaug

SEQ ID NO: 28 - RNA, 195 nt, Artificial Sequence
  Description of Artificial Sequence; note = synthetic construct
uuguucagaugaaguuagcgggagagcuuuggcuuuugccauacaccgaagaaguaaaucuuucagguaucuauuuaauuagagaugaccgcuauuggaugaacccuuggagagacucuuaaagagcaccgaaggagaaagcauaaaaaaagcgaaacucucagguaaaaggacaggggacagauaaaauaucuu

SEQ ID NO: 29 - RNA, 190 nt, Artificial Sequence
  Description of Artificial Sequence; note = synthetic construct
auguucggaugaagguaaugggagagugauauuuuaaauauccaccgaagagggaaaucuuucagguauuaggaccguuacuggacgagccucuggagagacucuuuuaaaaaagagcaccgaaggagcaaggucaaauuuuuugacugaaacucucagguaaaaggacagaggauaagguuaguuacuu

SEQ ID NO: 30 - RNA, 200 nt, Artificial Sequence
  Description of Artificial Sequence; note = synthetic construct
uauucuaguugaagaguauaagagagauccuauuuuaaaggacgccgaagggacaaucuauguuuaucccaauaaaacauagagaaauucucaggcaaaagaauuauacuuugauagacucuggaaaguaaacagagagagagcgaacgugggguuuguucucucuuuauuuuuuuaacagagaggacaaaccuuggggu

SEQ ID NO: 31 - RNA, 203 nt, Artificial Sequence
  Description of Artificial Sequence; note = synthetic construct
agguucggaugaagguaauaggagagaggucuuuugacccaccgaagaugcaaaaaucuuucagguaccauuguuuauggcgaggacuguuauuggacgaaacucuggagagacucuuuuuuauaaaagagcaccgaaggagcaaguuggguaaaaccaaugaaacucucagguaaaaggacagagcguagaagugaaguuua

SEQ ID NO: 32 - RNA, 126 nt, Artificial Sequence
  Description of Artificial Sequence; note = synthetic construct
uuagcgggugaauguaaacagagagacugugaaaagcagcgccgacggggaaagcauaaguuaugugaaacucucaggcaaaaggauguuuacgggacgcaacucuggagucauuuuuaugucacg

SEQ ID NO: 33 - RNA, 127 nt, Artificial Sequence
  Description of Artificial Sequence; note = synthetic construct
auuagcgggugaauguaagcagagagacugcgaaaagcagcgccgacggggaaagcauauauuaugugaaacucucaggcaaaaggauguuuacgggacgcaacucuggagucauuuuuguguuacg

SEQ ID NO: 34 - RNA, 215 nt, Artificial Sequence
  Description of Artificial Sequence; note = synthetic construct
aucagcggaagauuacaaggggagaguuuacaacgaauaguaacgccgaaggagcaagugaagagcgaaucucucaggccaaaaagacucuuguaugacgcaacucuggagaguguuuacgaagguaaaccacccacgaagcaaauauuuguucuuuuuugaagaaugaauaugcaacuuucugguauaaggacagagauuucuucacuauggag

SEQ ID NO: 35 - RNA, 133 nt, Artificial Sequence
  Description of Artificial Sequence; note = synthetic construct
aaaagcgagugaucaguauuagagagaauagagcguuaagacucuaucgccgaaggugcaaguaauuuauuacgaaacucucaggcaaaaggauaauacuguaacgcguuccugaauuggugauuuauaaaca

SEQ ID NO: 36 - RNA, 178 nt, Artificial Sequence
  Description of Artificial Sequence; note = synthetic construct
uuaagcgauugaucaguauuagagagaauagagaucaaaacuugaaucauuauaaguuaaacguuuuuagucuauuacaauuuugacucucuaucgccgaaggugcaagugaaauaacgaaacucucaggcaaaaggauaauacuguaacgcauuccugaaauguguauuaaaacagg

SEQ ID NO: 37 - RNA, 230 nt, Artificial Sequence
  Description of Artificial Sequence; note = synthetic construct
auaccgaaugacgucauucaggagaagaaaguuagacuuucgccgaaggaauuacacucucaggugucuuaagacaggacugauugacagacggacuucuggagagaccuauaaguagcaacaucuuuguauugacaccaagaugugcucuaggcgccgaaggggcaagaagaguaaaacaacuccuccaaucucucaggcaaaaggacagaagcuaaaagccaauauua

SEQ ID NO: 38 - RNA, 131 nt, Artificial Sequence
  Description of Artificial Sequence; note = synthetic construct
guaaaaccgagugacauuauuaggauaacugauaauugacggacuucuggagagaccuacuaggcgccgaaggggcaaggcuguuugcucaaacucucaggcaaaaggacagaaaagaaaaaaagaauuuu

SEQ ID NO: 39 - RNA, 134 nt, Artificial Sequence
  Description of Artificial Sequence; note = synthetic construct
aaguggggaaucguuugauuuuccaugacuguaaauggacggaacucuggagagaccguaaaggcaccgaaggggcaaggcaggcaacugcucaaacucucagguaaaaggacagagcuaggauagaccgcuuu

SEQ ID NO: 40 - RNA, 185 nt, Artificial Sequence
  Description of Artificial Sequence; note = synthetic construct
aaaccgaaugaugucaugcaggagaagaauuuuuuucgccgaaggaguuauacucucagguguucaguuuuugaacgggacuguuugauggacggacuucuggagagaccuuauuaggcgccgaaggggcaaggcauacugcucaaucucucaggcaaaaggacagaagguaaaauacaaacacc

SEQ ID NO: 41 - RNA, 222 nt, Artificial Sequence
  Description of Artificial Sequence; note = synthetic construct
cggccgcuugaauccgcgcgggagaguuccggguacgugugcccggacgccgaaggagcaagucccucccuugaaucucucaggccccguuaccgcgcgggcgaggcacaucugaaaagcgggccgcuguccaguggcuccacccaaggugcaagccagugacccgugacggucauggcgaaccucucagguuccgaugacagauggggaggaacgaccucg

SEQ ID NO: 42 - RNA, 229 nt, Artificial Sequence
  Description of Artificial Sequence; note = synthetic construct
auggcugcugaccccgugcgggagaguccuccggaagucgucggaggcgccgaaggagcaaauccuccccggaaucucucaggcacacguaccgcacggacgaggucacucuggaaagcagggcggaugucuauggcuuccgcucucaccgacggugaaagccggagcgcccucgggcggaccggcgaagcucucagguugagaugacagagggggaggccgucggggu

SEQ ID NO: 43 - RNA, 222 nt, Artificial Sequence
  Description of Artificial Sequence; note = synthetic construct
cugucuagaugaagauagcgagagauuaucuuccauaaauggaagauagccgaaggggaaauacaaaggcccgccaagccuuuguagaagcucucaggcggcaggaucgcuaucggauaggccucuggaaagucucguaaagagcaccgaaggagcaauacauauggaaggccauauguagaagcucucagguagaaaaacagaggaguugugauggcacuu

SEQ ID NO: 44 - RNA, 206 nt, Artificial Sequence
  Description of Artificial Sequence; note = synthetic construct
gaaaugggaugaagaaugcgggagagacccuaaccgggcgccgaaggagcaagcggguauauggccuguauacucgugaaacucucaggcaaaaggaccgcauucggaccauaucccggaaagccucuaaagaggcaccgaaggagcaauucuucuauaaagaagaaucucucagguaaacagacgggggaauaaaaggcguaagg

SEQ ID NO: 45 - RNA, 191 nt, Artificial Sequence
  Description of Artificial Sequence; note = synthetic construct
ugagcgucgcaucaucguugggagaaaccgcuucauugcggugccgaaggagcaaccgccccggaaacucucaggcaaaaggaccagcgaugacgacggaacucuggagagaagccaccuugacuaaaggacggcucgccgaagggauaacaaucucaggcgacaaggacagagggggcucuugaaccggc

SEQ ID NO: 46 - RNA, 202 nt, Artificial Sequence
  Description of Artificial Sequence; note = synthetic construct
uguaucguacggccacgucgggagagaccggcuuuaggcagccggcgccgaaggagcaaccgccccggaaacucucaggcaaaaggaccgcguggcuuugacagcaucuggaaagaggcgccgacgagcuugaggcucggacagcguccgccgacgggauaauacucucaggcacagcgacagauggggcuucgacugguug

SEQ ID NO: 47 - RNA, 203 nt, Artificial Sequence
  Description of Artificial Sequence; note = synthetic construct
ucuugcacugucuguuugcgggagagagccguuaaggccgccgaaggggaaaacgcccgaaaucucucagguacaaggaaccgcaggcggguaagacaacucuggaaagucgggggcaacuccgcgccgaagguguaaguauggcuuuauauauagccaugcgagucucucaggccugagacagaggggcacgaaccaaccgc

SEQ ID NO: 48 - RNA, 203 nt, Artificial Sequence
  Description of Artificial Sequence; note = synthetic construct
ucuugcacugucuguuugcgggagagagccguuaaggccgccgaaggggaaaacgcccgaaaucucucagguacaaggaaccgcaggcggguaagacaacucuggaaagucgggggcaacuccgcgccgaagguguaaguauggcuuuauauauagccaugcgagucucucaggccugagacagaggggcacgaaccaacugc

SEQ ID NO: 49 - RNA, 224 nt, Artificial Sequence
  Description of Artificial Sequence; note = synthetic construct
aagagcccucgacccucgcgggagacaucgggauucgaucccgaggccgaaggcgcaaccgccccggaaacgcucaggcaaaaggaccgcgcggguuuaggaacgcuggaaagcagucucuccaccggaggggcucgccgaaggagcaaggccaaacccguccggcagaggggcgaggccggaaucucucaggcccaagggacagcgggggcgacuugccggcg

SEQ ID NO: 50 - RNA, 213 nt, Artificial Sequence
  Description of Artificial Sequence; note = synthetic construct
uuccucaaaggccucucgcgggagagaucgggccuugcccggcgcugaaggcgaaaccgccccggaaacgcucaagcagaaggaccgcgcgagacguugaacgcuggaaagcagagcgcgcgccgcucucgccgaaggagcaaggccugccgacugucuggucggccgcugaaucucucaggcgccaaggacagcgggggcagaaggcggguc

SEQ ID NO: 51 - RNA, 208 nt, Artificial Sequence
  Description of Artificial Sequence; note = synthetic construct
ucgaguccuaccuguuugcgggagagagcagcgagagcugccgccgaaggggaaaucgcccgaaaucucucaggcaaaagaaccguagacgggaaagacacucuggaaagucggggcuugcccccgcgccgaagguguaagcgccgcugacagaguuccgguugcgcgagucucucaggcuucagacagaggggcacggacuggucgc

SEQ ID NO: 52 - RNA, 197 nt, Artificial Sequence
  Description of Artificial Sequence; note = synthetic construct
gguguucaaaggcuggacgggagagaucggcuacugccgacgccgacggagcaaccgaucuggagagagacgccucgagcguccaccgaaggggaaagccggcaggcccgguuaagcucucagguagccgagacagauuuggggauuucgcugcgccccaaggaaacucucaggcaaaagugaccgcaccgccgaac

SEQ ID NO: 53 - RNA, 180 nt, Artificial Sequence
  Description of Artificial Sequence; note = synthetic construct
aucuggauucgaccucguugggagaaaccgguucgauccggugccgaaggagcaaccgccccggaaacucucaggccaaaggaccagcaaggugccgguaggacucuggagagaagcguucgcgcucgccgaagggauaacaaucucaggcaaagggacagagggggcucgaauuugucg

SEQ ID NO: 54 - RNA, 211 nt, Artificial Sequence
  Description of Artificial Sequence; note = synthetic construct
uccaccaacgugcaguucgggagagaccguccagcggacggcgccgacggagcaaccaccccggaaacucucaggcaaaaggaccgaccugcuggaacaucuggagaguggcgcgcgguacggcgcccaccgaaggggaucccuggcgcguuugcagcggcgcacgggugaagcucucagguaaauggacagaugggguugcggccgggcc

SEQ ID NO: 55 - RNA, 251 nt, Artificial Sequence
  Description of Artificial Sequence; note = synthetic construct
gcgcugucacgcaugucgcgggagagagcggccgauugcggcugccgccgaaggcgcaauucgcccggaaucgcucagguaacccauaccgcgacugcaucgaguagcgccuggcgcgcucgaaacagcacucuggagagaccuggcgcggccaccuucgcggugcgcaagcagcccaggcgccgaaggugcaaacccgcucgcgcggggcaacucucaggcaaaaggacagaggggcggaaaauucgacc

SEQ ID NO: 56 - RNA, 212 nt, Artificial Sequence
  Description of Artificial Sequence; note = synthetic construct
ruccaccaacgugcaguucgggagagaccguccagcggacggcgccgacggagcaaccaccccggaaacucucaggcaaaaggaccgaccugcuggaacaucuggagaguggcgcgcgguacggcgcccaccgaaggggaucccuggcgcguuugcagcggcgcacgggugaagcucucagguaaauggacagaugggguugcggccgggcc

SEQ ID NO: 57 - RNA, 251 nt, Artificial Sequence
  Description of Artificial Sequence; note = synthetic construct
gcgcugucacgcaugucgcgggagagagcggccgauugcggcugccgccgaaggcgcaauucgcccggaaucgcucagguaacccauaccgcgacugcaucgaguagcgccuggcgcgcucgaaacagcacucuggagagaccuggcguggccaccuucgcggugcgcaagcagcccaggcgccgaaggugcaaacccgcucgcgcggggcaacucucaggcaaaaggacagaggggcggaaaauucgacc

SEQ ID NO: 58 - RNA, 251 nt, Artificial Sequence; identical to SEQ ID NO: 55
  SEQ ID NO59

SEQ ID NO: 59 - RNA, 206 nt, Artificial Sequence
  Description of Artificial Sequence; note = synthetic construct
gcuaauauuccgguucugcgggagagaggggccacgcccccgccgaagacgcaagcucccauaaucgcucaggcaaccguaccgcagcgccguauagaaucaagccgauuggagagaggccgccccgcgcggcccaccgaaggggcaaguggccuaaggccgcgcaacucucagguaaaaaggacaaggggagaggcuguuaccca

SEQ ID NO: 60 - RNA, 221 nt, Artificial Sequence
  SEQ ID NO60
cauaaucgccgggucauacaggagagagcggcuuucuggccgccgccgaaggcgcaagcgcacccgcaaucgcucaggcaaaaggacuguaucaucgggccggcggcagccgguucgcgcaaucuggagagcggcguccgcgcgacgcccaccgaaggggcuaacggcuuauccggccggaaaaucucaggugcagggacagaggggugugguugaugagg

SEQ ID NO: 61 - RNA, 271 nt, Artificial Sequence
  Description of Artificial Sequence; note = synthetic construct
acauaaucgggaaaugugcaggagaguguuacacccaacuacaauguaaccaccgaaggcgcagacacccuuaaaucgcucagguaucagggacugcacauugaaacaaacaaucuggagagcggcguuggaauaacguccaccgaaggggagaaggccgucugaaccaccauucagacaaccgcgcaaagcagugagcagacugguuugccaucaugcggauacagccgaaaaucucagguucaaggacagauagggucauccgcgcaca

SEQ ID NO: 62 - RNA, 220 nt, Artificial Sequence
  Description of Artificial Sequence; note = synthetic construct
gcugauauuucauccaggcaggagagugcgcuguaucaaaauacagugucaccgaaggcguaaccccccggaaucgcucagguguggccauaaggcuugaguaacugcuugugauuggcaaucuggagagugcugaaacagcuucagccaccgaaggggcaugcggaaaguugaccguaaaacucucagguaaaaggacagagggguaaguugauaucug

SEQ ID NO: 63 - RNA, 252 nt, Artificial Sequence
  Description of Artificial Sequence; note = synthetic construct
acauuccggccauccauccgggagagcgcgucggccgcauacggccagucgccgccgaagggguagcacccgaaaacucucaggcauccaggaccggguggaucggcugggcaugcguccggccgcacucuggagagcggcgccgcgucaccaucgcgggcgcccaccgaaggggcucacggaaccgucgcacgcguugugucgcuucccaaucucucagguaccaaggacagaggggccacccgcgcagca

SEQ ID NO: 64 - RNA, 219 nt, Artificial Sequence
  Description of Artificial Sequence; note = synthetic construct
acguucggaugaagguggcuggagaagaguuuaggcucuaccgacgagguaaaaucuuucaggugcaauaguuuaacuaugugaggacaguuacuggacgaacccuuggagagauccacucuguacuauaguacagaaaauggacgccgaaggcgcaaauauaaagcaaauuuaugugaaacgcucaggcaaaaggacaggggagaaaagugauuucca

SEQ ID NO: 65 - RNA, 208 nt, Artificial Sequence
  Description of Artificial Sequence; note = synthetic construct
gaguucggaugaagguagcuggagagcgggaaauugaccccacaccgacgauguaaaaucuuucaggugcgauggcagggacuguuacuggacgaacccuuggagagauccauuuuagaaauggacgccgaagcgcaaaagagcgguuaauuuuucaaucguuuuucaaacgcucaggcaaaaggacaggggcaaaagacaaucuauu

SEQ ID NO: 66 - RNA, 225 nt, Artificial Sequence
  Description of Artificial Sequence; note = synthetic construct
aaguucggaugaagguagcuggagaguagggaauugacccuacgccgacgagguaaacucuuucaggcgcugauuauaagcagggacuguuacuggacgaacccuuggagagagccguuauauuaaaaagagauaaaacggccgccgaaggcgcaaaaagagcgguuaauuuuuccguuuuuucaaacgcucaggcaaaaggacaggggcaacaagauauucggu

SEQ ID NO: 67 - RNA, 256 nt, Artificial Sequence
  Description of Artificial Sequence; note = synthetic construct
ccguucggaugaagguagcaggagaguggggaauuaaccccacaccgacgaggcaacuuuguuguuggaagcauuccauaaacaaagcacucuuucaggugccgcaaggcguggacuguuacuggacgagccucuggagagacuaccgauugcauuaacuugcaauaauagguggcgccgaaggcgaaagugucgcuguguuguuaagcgacgcgaaacgcucaggcaaaaggacagaggagaggaugacugcaca

SEQ ID NO: 68 - RNA, 248 nt, Artificial Sequence
  Description of Artificial Sequence; note = synthetic construct
uuguuccguugaagacugcaggagagugguuguuaaccagauuuuaacaucugagccaaauaacccgccgaagaaguaaaucuuucaggugcauuauucuuagccauauauuggcaacgaauaagcgaggacuguaguuggaggaaccucuggagagaaccguuuaaucggucgccgaaggagcaagcucugcgcauaugcagagugaaacucucaggcaaaaggacagaggagugaaaggccaaucu

SEQ ID NO: 69 - RNA, 228 nt, Artificial Sequence
  Description of Artificial Sequence; note = synthetic construct
auguuccgaugaagacugcaggagagugguuauuaaccaaacuuuaacauuugguuagauuaacucgccgaagaauuaacuauuucaggugcuaccuugguagcggggacuguaguuggaggaaccucuggagagaaccguuaaaucggucgccgaaggagcaaguccugcacaugugugcggggugaaacucucaggcaaaaggacagaggaguggaaaguuacaac

SEQ ID NO: 70 - RNA, 247 nt, Artificial Sequence
  Description of Artificial Sequence; note = synthetic construct
uuguuccgaugaaggcuacaggagagugguaauuaaccauauuuuaacauuugguuagucauacccgccgaagaaguaaaucuuucaggugcaauauucuuauugguuauaucaagagaauaugcgaggacuguaguuggaggaaccucuggagagaaccguuaaaucggucgccgaaggagcaaguccugcccaugugcagggugaaacucucaggcaaaaggacagaggaguggaaaguuauacc

SEQ ID NO: 71 - RNA, 209 nt, Artificial Sequence
  Description of Artificial Sequence; note = synthetic construct
aucggcccgcaacacggugggagaagcggcacugccgcugccgaaggcgcaacagcccguaaucgcucaggcccgauaccauccgcaguacaacucuggagagaccggccgaugccggcgccgaaggggcacgaaacgcaggcaggccacgcgccaggccgcguuuuuaaacucucaggcaaaaggacagaggggcgcgaggaagaccg

SEQ ID NO: 72 - RNA, 210 nt, Artificial Sequence
  Description of Artificial Sequence; note = synthetic construct
aaucggcagguaacacggugggagaagcggcacugccgcugccgaaggcgcaacagcccguaaucgcucaggcccgauaccaucuucaacacaacucuggagagaccgguucacuccggcgccgaaggggcacggaacgcaggcaggccacaggccaggccgcguucuuaaacucucaggcaaaaggacagaggggcgcgaggaagaccg

SEQ ID NO: 73 - RNA, 260 nt, Artificial Sequence
  Description of Artificial Sequence; note = synthetic construct
aagcauuucauggggugcgucgagagaucgagaacggcucgguuuaauccuauucgggugcguggcugaagcuauaaucaccucgcuucauggcgaggaaggcgcaagguucgucauugcucagcuugauguuauucgcagcacaccuguggagacgccagugcuaugccguguugaaggggugcgagaagcaggcggauuggcgcuugcuugguuuucaaagucucaggcaaaaggagagagcgauaugaagauugcuc

SEQ ID NO: 74 - RNA, 217 nt, Artificial Sequence
  Description of Artificial Sequence; note = synthetic construct
acagccgcugacgcgaugugggagaaccuccaugucgaggcgccguaggagcaaucuccuccccgagaaucucucaggcccaagcaccacaccgccgaggcaacucuggagacagggacggucgcaccgaccgugccugaccgaagguguagagcggcgccaugaugcgacgccgcagacucucagguuucaggacagagcggggagggcgcaacca

SEQ ID NO: 75 - RNA, 249 nt, Artificial Sequence
  Description of Artificial Sequence; note = synthetic construct
caacgcaucgcucuagugcgggagaguucuguggcugccagcuacggacgccgaaggagcaauaccucuccgucaaccucucaggcacccggaccgcgcgagacuacgaugccucuggaaagcgguggcgaccccuggcgguccucacccgccgauggggaaaggcgauucaccugacgguggacagagucgccgaaucucucaggcgccuggcgugcaggugaagacagagggagagggccgcuaguc

SEQ ID NO: 76 - RNA, 217 nt, Artificial Sequence; identical to SEQ ID NO: 74
  Description of Artificial Sequence; note = synthetic construct

SEQ ID NO: 77 - RNA, 248 nt, Artificial Sequence
  Description of Artificial Sequence; note = synthetic construct
aacgcaucgcucuagugcgggagaguucuguggcugccagcuacggacgccgaaggagcaauaccucuccgucaaccucucaggcacccggaccgcgcgagacuacgaugccucuggaaagcgguggcgaccccuggcgguccucacccgccgauggggaaaggcgauucaccugacgguggacagagucgccgaaucucucaggcgccuggcgugcaggugaagacagagggagagggccgcuaguc

SEQ ID NO: 78 - RNA, 232 nt, Artificial Sequence
  Description of Artificial Sequence; note = synthetic construct
acggcugcugaccccgcgcgggagaguccuccggacaucaccggaggcgccgaaggagcaaauccuccccggaaucucucaggcucacguaccgcacggacgaggucacucuggaaagcagggcgggugucgacggcuuccgcucucaccgacggugaaagccgggcagagcuccagggcucgcccggugaagcucucagguugagaugacagagggggaggccguccgggu

SEQ ID NO: 79 - RNA, 227 nt, Artificial Sequence
  Description of Artificial Sequence; note = synthetic construct
cggccguuugaauccgcgcgggagaguccccggccgcgccggggcgccgaaggagcaagucccucccuugaaucucucaggcaccguuaccgcgcgggcgaggcacaucugaaaagcggaccgcccccgacggcggucccacccaaggugcaagcccugaucgccguacuccgguggccguggcgaaccucucagguuccgaugacagauggggaggaccgaccucg

SEQ ID NO: 80 - RNA, 188 nt, Artificial Sequence
  Description of Artificial Sequence; note = synthetic construct
auaaucggaugaagauaugaggagagauuucauuuuaaugaaacaccgaagaaguaaaucuuucagguaaaaaggacucauauuggacgaaccucuggagagcuuaucuaagagauaacaccgaaggagcaaagcuaauuuuagccuaaacucucagguaaaaggacggaguaauugugcaauuuaua

(19) United States
(12) Patent Application Publication
(10) Pub. No.: US 2013/0029342 A1
(43) Pub. Date: Jan. 31, 2013

(54) GLYCINE RIBOSWITCHES, METHODS FOR THEIR USE, AND COMPOSITIONS FOR USE WITH GLYCINE RIBOSWITCHES

(71) Applicants: Ronald R. Breaker, Guilford, CT (US); Jeffrey Barrick, Lansing, MI (US); Maumita Mandal, Fremont, CA (US)

(72) Inventors: Ronald R. Breaker, Guilford, CT (US); Jeffrey Barrick, Lansing, MI (US); Maumita Mandal, Fremont, CA (US)

(73) Assignee: Yale University

(21) Appl. No.: 13/563,407

(22) Filed: Jul. 31, 2012

Related U.S. Application Data
(62) Division of application No. 11/664,655, filed on Dec. 6, 2007.
(60) Provisional application No. 60/617,309, filed on Oct. 7, 2004.

(51) Int. Cl. C12N 15/113; C12Q 1/68; C12N 15/63

(52) U.S. Cl. 435/6.12; 435/320.1; 536/24.1

(57) ABSTRACT

Riboswitches are structural elements in mRNA that change state when bound by a trigger molecule, and are thus able to regulate gene expression. They can be dissected into two separate domains: one that selectively binds the target (aptamer domain) and another that influences genetic control (expression platform domain). Bacterial glycine riboswitches consist of two tandem aptamer domains which cooperatively bind glycine to regulate the expression of downstream genes. These natural switches are targets for antibiotics and other small molecule therapies. Modified versions of these natural riboswitches can be employed as designer genetic switches that are controlled by specific effector compounds. Disclosed are isolated and recombinant riboswitches, and compositions and methods for selecting and identifying compounds that can activate, inactivate, or block a riboswitch.

### CROSS-REFERENCE TO RELATED APPLICATIONS

This application is a divisional of application Ser. No. 11/664,655, filed Dec. 6, 2007, which claims benefit of U.S. Provisional Application No. 60/617,309, filed Oct. 7, 2004. U.S. application Ser. No. 11/664,655, filed Dec. 6, 2007, and U.S. Provisional Application No. 60/617,309, filed Oct. 7, 2004, are hereby incorporated herein by reference in their entirety.

### REFERENCE TO SEQUENCE LISTING

The Sequence Listing submitted Jul. 31, 2012 as a text file named “YU_{—}8_{—}8403_AMD_AFD_Sequence_Listing.txt,” created on Jul. 31, 2012, and having a size of 34,213 bytes is hereby incorporated by reference pursuant to 37 C.F.R. §1.52(e)(5).

### STATEMENT REGARDING FEDERALLY SPONSORED RESEARCH

This invention was made with government support under Grants NIH 1024197-1-A05274-615002 awarded by the National Institutes of Health, and Grant 1024351-1-D01084-615002 awarded by the National Science Foundation. The government has certain rights in the invention.

### FIELD OF THE INVENTION

The disclosed invention is generally in the field of gene expression and specifically in the area of regulation of gene expression.

### BACKGROUND OF THE INVENTION

Precision genetic control is an essential feature of living systems, as cells must respond to a multitude of biochemical signals and environmental cues by varying genetic expression patterns. Most known mechanisms of genetic control involve the use of protein factors that sense chemical or physical stimuli and then modulate gene expression by selectively interacting with the relevant DNA or messenger RNA sequence. Proteins can adopt complex shapes and carry out a variety of functions that permit living systems to sense accurately their chemical and physical environments. Protein factors that respond to metabolites typically act by binding DNA to modulate transcription initiation (e.g. the lac repressor protein; Matthews, K. S., and Nichols, J. C., 1998, Prog. Nucleic Acids Res. Mol. Biol. 58, 127-164) or by binding RNA to control either transcription termination (e.g. the PyrR protein; Switzer, R. L., et al., 1999, Prog. Nucleic Acids Res. Mol. Biol. 62, 329-367) or translation (e.g. the TRAP protein; Babitzke, P., and Golinick, P., 2001, J. Bacteriol. 183, 5795-5802). Protein factors responds to environmental stimuli by various mechanisms such as allosteric modulation or post-translational modification, and are adept at exploiting these mechanisms to serve as highly responsive genetic switches (e.g. see Ptashne, M., and Gann, A. (2002). Genes and Signals. Cold Spring Harbor Laboratory Press, Cold Spring Harbor, N.Y.).

In addition to the widespread participation of protein factors in genetic control, it is also known that RNA can take an active role in genetic regulation. Recent studies have begun to reveal the substantial role that small non-coding RNAs play in selectively targeting mRNAs for destruction, which results in down-regulation of gene expression (e.g. see Hannon, G. J. 2002, Nature 418, 244-251 and references therein). This process of RNA interference takes advantage of the ability of short RNAs to recognize the intended mRNA target selectively via Watson-Crick base complementation, after which the bound mRNAs are destroyed by the action of proteins. RNAs are ideal agents for molecular recognition in this system because it is far easier to generate new target-specific RNA factors through evolutionary processes than it would be to generate protein factors with novel but highly specific RNA binding sites.

Although proteins most requirements that biology has for enzyme, receptor and structural functions, RNA also can serve in these capacities. For example, RNA has sufficient structural plasticity to form numerous ribozyme domains (Cech & Golden, Building a catalytic active site using only RNA. In: The RNA World R. F. Gesteland, T. R. Cech, J. F. Atkins, eds., pp. 321-350 (1998); Breaker, In vitro selection of catalytic polynucleotides. Chem. Rev. 97, 371-390 (1997)) and receptor domains (Osborne & Ellington, Nucleic acid selection and the challenge of combinatorial chemistry. Chem. Rev. 97, 349-370 (1997); Hermann & Patel, Adaptive recognition by nucleic acid aptamers, Science 287, 820-825 (2000)) that exhibit considerable enzymatic power and precise molecular recognition. Furthermore, these activities can be combined to create allosteric ribozymes (Soukup & Breaker, Engineering precision RNA molecular switches. Proc. Natl. Acad. Sci. USA 96, 3584-3589 (1999); Seetharaman et al., Immobilized riboswitches for the analysis of complex chemical and biological mixtures. Nature Biotechnol. 19, 336-341 (2001)) that are selectively modulated by effector molecules.

These properties of RNA are consistent with speculation (Gold et al., From oligonucleotide shapes to genomic SELEX: novel biological regulatory loops. Proc. Natl. Acad. Sci. USA 94, 59-64 (1997); Gold et al., SELEX and the evolution of genomes. Curr. Opin. Gen. Dev. 7, 848-851 (1997); Nou & Kadner, Adenosylcobalamin inhibits ribosome binding to btuB RNA. Proc. Natl. Acad. Sci. USA 97, 7190-7195 (2000); Gelfand et al., A conserved RNA structure element involved in the regulation of bacterial riboflavin synthesis genes. Trends Gen. 15, 439-442 (1999); Miranda-Rios et al., A conserved RNA structure (thi box) is involved in regulation of thiamin biosynthetic gene expression in bacteria. Proc. Natl. Acad. Sci. USA 98, 9736-9741 (2001); Stormo & Ji, Do mRNAs act as direct sensors of small molecules to control their expression?Proc. Natl. Acad. Sci. USA 98, 9465-9467 (2001)) that certain mRNAs might employ allosteric mechanisms to provide genetic regulatory responses to the presence of specific metabolites. Although a thiamine pyrophosphate (TPP)-dependent sensor/regulatory protein had been proposed to participate in the control of thiamine biosynthetic genes (Webb & Downs, Characterization of thiL, encoding thiamin-monophosphate kinase, in Salmonella typhimurium. J. Biol. Chem. 272, 15702-15707 (1997)), no such protein factor has been shown to exist.

Transcription of the lysC gene of B. subtilis is repressed by high concentrations of lysine (Kochhar, S., and Paulus, H. 1996, Microbiol. 142:1635-1639; Mäder, U., et al., 2002, J. Bacteriol. 184:4288-4295; Patte, J. C. 1996. Biosynthesis of lysine and threonine. In: Escherichia coli and Salmonella: Cellular and Molecular Biology, F. C. Neidhardt, et al., eds., Vol. 1, pp. 528-541. ASM Press, Washington, D.C.; Patte, J.-C., et al., 1998, FEMS Microbiol. Lett. 169:165-170), but that no protein factor had been identified that served as the genetic regulator (Liao, H.-H., and Hseu, T.-H. 1998, FEMS Microbiol. Lett. 168:31-36). The lysC gene encodes aspartokinase II, which catalyzes the first step in the metabolic pathway that converts L-aspartic acid into L-lysine (Belitsky, B. R. 2002. Biosynthesis of amino acids of the glutamate and aspartate families, alanine, and polyamines. In: Bacillus subtilis and its Closest Relatives: from Genes to Cells. A. L. Sonenshein, J. A. Hoch, and R. Losick, eds., ASM Press, Washington, D.C.).

### BRIEF SUMMARY OF THE INVENTION

It has been discovered that certain natural mRNAs serve as metabolite-sensitive genetic switches wherein the RNA directly binds a small organic molecule. This binding process changes the conformation of the mRNA, which causes a change in gene expression by a variety of different mechanisms. Modified versions of these natural “riboswitches” (created by using various nucleic acid engineering strategies) can be employed as designer genetic switches that are controlled by specific effector compounds. Such effector compounds that activate a riboswitch are referred to herein as trigger molecules. The natural switches are targets for antibiotics and other small molecule therapies. In addition, the architecture of riboswitches allows actual pieces of the natural switches to be used to construct new non-immunogenic genetic control elements, for example the aptamer (molecular recognition) domain can be swapped with other non-natural aptamers (or otherwise modified) such that the new recognition domain causes genetic modulation with user-defined effector compounds. The changed switches become part of a therapy regimen-turning on, or off, or regulating protein synthesis. Newly constructed genetic regulation networks can be applied in such areas as living biosensors, metabolic engineering of organisms, and in advanced forms of gene therapy treatments.

Riboswitches can have single or multiple aptamer domains. Aptamer domains in riboswitches having multiple aptamer domains can exhibit cooperative binding of trigger molecules or can not exhibit cooperative binding of trigger molecules. In the latter case, the aptamer domains can be said to be independent binders. Riboswitches having multiple aptamers can have one or multiple expression platform domains. For example, a riboswitch having two aptamer domains that exhibit cooperative binding of their trigger molecules can be linked to a single expression platform domain that is regulated by both aptamer domains. Riboswitches having multiple aptamers can have one or more of the aptamers joined via a linker. Where such aptamers exhibit cooperative binding of trigger molecules, the linker can be a cooperative linker.

Disclosed are isolated and recombinant riboswitches, recombinant constructs containing such riboswitches, heterologous sequences operably linked to such riboswitches, and cells and transgenic organisms harboring such riboswitches, riboswitch recombinant constructs, and riboswitches operably linked to heterologous sequences. The heterologous sequences can be, for example, sequences encoding proteins or peptides of interest, including reporter proteins or peptides. Preferred riboswitches are, or are derived from, naturally occurring riboswitches.

Also disclosed are chimeric riboswitches containing heterologous aptamer domains and expression platform domains. That is, chimeric riboswitches are made up an aptamer domain from one source and an expression platform domain from another source. The heterologous sources can be from, for example, different specific riboswitches or different classes of riboswitches. The heterologous aptamers can also come from non-riboswitch aptamers. The heterologous expression platform domains can also come from non-riboswitch sources.

Also disclosed are compositions and methods for selecting and identifying compounds that can activate, deactivate or block a riboswitch. Activation of a riboswitch refers to the change in state of the riboswitch upon binding of a trigger molecule. A riboswitch can be activated by compounds other than the trigger molecule and in ways other than binding of a trigger molecule. The term trigger molecule is used herein to refer to molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques). Non-natural trigger molecules can be referred to as non-natural trigger molecules.

Deactivation of a riboswitch refers to the change in state of the riboswitch when the trigger molecule is not bound. A riboswitch can be deactivated by binding of compounds other than the trigger molecule and in ways other than removal of the trigger molecule. Blocking of a riboswitch refers to a condition or state of the riboswitch where the presence of the trigger molecule does not activate the riboswitch.

Also disclosed are compounds, and compositions containing such compounds, that can activate, deactivate or block a riboswitch. Also disclosed are compositions and methods for activating, deactivating or blocking a riboswitch. Riboswitches function to control gene expression through the binding or removal of a trigger molecule. Compounds can be used to activate, deactivate or block a riboswitch. The trigger molecule for a riboswitch (as well as other activating compounds) can be used to activate a riboswitch. Compounds other than the trigger molecule generally can be used to deactivate or block a riboswitch. Riboswitches can also be deactivated by, for example, removing trigger molecules from the presence of the riboswitch. A riboswitch can be blocked by, for example, binding of an analog of the trigger molecule that does not activate the riboswitch.

Also disclosed are compositions and methods for altering expression of an RNA molecule, or of a gene encoding an RNA molecule, where the RNA molecule includes a riboswitch, by bringing a compound into contact with the RNA molecule. Riboswitches function to control gene expression through the binding or removal of a trigger molecule. Thus, subjecting an RNA molecule of interest that includes a riboswitch to conditions that activate, deactivate or block the riboswitch can be used to alter expression of the RNA. Expression can be altered as a result of, for example, termination of transcription or blocking of ribosome binding to the RNA. Binding of a trigger molecule can, depending on the nature of the riboswitch, reduce or prevent expression of the RNA molecule or promote or increase expression of the RNA molecule.

Also disclosed are compositions and methods for regulating expression of an RNA molecule, or of a gene encoding an RNA molecule, by operably linking a riboswitch to the RNA molecule. A riboswitch can be operably linked to an RNA molecule in any suitable manner, including, for example, by physically joining the riboswitch to the RNA molecule or by engineering nucleic acid encoding the RNA molecule to include and encode the riboswitch such that the RNA produced from the engineered nucleic acid has the riboswitch operably linked to the RNA molecule. Subjecting a riboswitch operably linked to an RNA molecule of interest to conditions that activate, deactivate or block the riboswitch can be used to alter expression of the RNA.

Also disclosed are compositions and methods for regulating expression of a naturally occurring gene or RNA that contains a riboswitch by activating, deactivating or blocking the riboswitch. If the gene is essential for survival of a cell or organism that harbors it, activating, deactivating or blocking the riboswitch can in death, stasis or debilitation of the cell or organism. For example, activating a naturally occurring riboswitch in a naturally occurring gene that is essential to survival of a microorganism can result in death of the microorganism (if activation of the riboswitch turns off or represses expression). This is one basis for the use of the disclosed compounds and methods for antimicrobial and antibiotic effects.

Also disclosed are compositions and methods for regulating expression of an isolated, engineered or recombinant gene or RNA that contains a riboswitch by activating, deactivating or blocking the riboswitch. The gene or RNA can be engineered or can be recombinant in any manner. For example, the riboswitch and coding region of the RNA can be heterologous, the riboswitch can be recombinant or chimeric, or both. If the gene encodes a desired expression product, activating or deactivating the riboswitch can be used to induce expression of the gene and thus result in production of the expression product. If the gene encodes an inducer or repressor of gene expression or of another cellular process, activation, deactivation or blocking of the riboswitch can result in induction, repression, or de-repression of other, regulated genes or cellular processes. Many such secondary regulatory effects are known and can be adapted for use with riboswitches. An advantage of riboswitches as the primary control for such regulation is that riboswitch trigger molecules can be small, non-antigenic molecules.

Also disclosed are compositions and methods for altering the regulation of a riboswitch by operably linking an aptamer domain to the expression platform domain of the riboswitch (which is a chimeric riboswitch). The aptamer domain can then mediate regulation of the riboswitch through the action of, for example, a trigger molecule for the aptamer domain. Aptamer domains can be operably linked to expression platform domains of riboswitches in any suitable manner, including, for example, by replacing the normal or natural aptamer domain of the riboswitch with the new aptamer domain. Generally, any compound or condition that can activate, deactivate or block the riboswitch from which the aptamer domain is derived can be used to activate, deactivate or block the chimeric riboswitch.

Also disclosed are compositions and methods for inactivating a riboswitch by covalently altering the riboswitch (by, for example, crosslinking parts of the riboswitch or coupling a compound to the riboswitch). Inactivation of a riboswitch in this manner can result from, for example, an alteration that prevents the trigger molecule for the riboswitch from binding, that prevents the change in state of the riboswitch upon binding of the trigger molecule, or that prevents the expression platform domain of the riboswitch from affecting expression upon binding of the trigger molecule.

Also disclosed are methods of identifying compounds that activate, deactivate or block a riboswitch. For examples, compounds that activate a riboswitch can be identified by bringing into contact a test compound and a riboswitch and assessing activation of the riboswitch. If the riboswitch is activated, the test compound is identified as a compound that activates the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

Identification of compounds that block a riboswitch can be accomplished in any suitable manner. For example, an assay can be performed for assessing activation or deactivation of a riboswitch in the presence of a compound known to activate or deactivate the riboswitch and in the presence of a test compound. If activation or deactivation is not observed as would be observed in the absence of the test compound, then the test compound is identified as a compound that blocks activation or deactivation of the riboswitch.

Also disclosed are biosensor riboswitches. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a riboswitch that includes a conformation dependent label, the signal from which changes depending on the activation state of the riboswitch. Such a biosensor riboswitch preferably uses an aptamer domain from or derived from a naturally occurring riboswitch. Also disclosed are methods of detecting compounds using biosensor riboswitches. The method can include bringing into contact a test sample and a biosensor riboswitch and assessing the activation of the biosensor riboswitch. Activation of the biosensor riboswitch indicates the presence of the trigger molecule for the biosensor riboswitch in the test sample.

Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

Also disclosed are methods for selecting, designing or deriving new riboswitches and/or new aptamers that recognize new trigger molecules. Such methods can involve production of a set of aptamer variants in a riboswitch, assessing the activation of the variant riboswitches in the presence of a compound of interest, selecting variant riboswitches that were activated (or, for example, the riboswitches that were the most highly or the most selectively activated), and repeating these steps until a variant riboswitch of a desired activity, specificity, combination of activity and specificity, or other combination of properties results. Also disclosed are riboswitches and aptamer domains produced by these methods.

The disclosed riboswitches, including the derivatives and recombinant forms thereof, generally can be from any source, including naturally occurring riboswitches and riboswitches designed de novo. Any such riboswitches can be used in or with the disclosed methods. However, different types of riboswitches can be defined and some such sub-types can be useful in or with particular methods (generally as described elsewhere herein). Types of riboswitches include, for example, naturally occurring riboswitches, derivatives and modified forms of naturally occurring riboswitches, chimeric riboswitches, and recombinant riboswitches. A naturally occurring riboswitch is a riboswitch having the sequence of a riboswitch as found in nature. Such a naturally occurring riboswitch can be an isolated or recombinant form of the naturally occurring riboswitch as it occurs in nature. That is, the riboswitch has the same primary structure but has been isolated or engineered in a new genetic or nucleic acid context. Chimeric riboswitches can be made up of, for example, part of a riboswitch of any or of a particular class or type of riboswitch and part of a different riboswitch of the same or of any different class or type of riboswitch; part of a riboswitch of any or of a particular class or type of riboswitch and any non-riboswitch sequence or component. Recombinant riboswitches are riboswitches that have been isolated or engineered in a new genetic or nucleic acid context.

Different classes of riboswitches refer to riboswitches that have the same or similar trigger molecules or riboswitches that have the same or similar overall structure (predicted, determined, or a combination). Riboswitches of the same class generally, but need not, have both the same or similar trigger molecules and the same or similar overall structure. Riboswitch classes include glycine-responsive riboswitches, guanine-responsive riboswitch, adenine-responsive riboswitch, lysine-responsive riboswitch, thiamine pyrophosphate-responsive riboswitch, adenosylcobalamin-responsive riboswitch, flavin mononucleotide-responsive riboswitch, and a S-adenosylmethionine-responsive riboswitch.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1D show the structure and properties of a glycine-responsive riboswitch from Vibrio cholerae. The sequence in FIG. 1A is SEQ ID NO: 1. The sequences in FIG. 1B are GGGUUGAAGACUGCAGCAGAGUGCGUUGUUAA CCAGAUUUUAACAUCUGACGCCAAAUAACCCGCCGAAGAAGUAAAUCUUUA CGGUGCAUUAUUCUUAGCCAAUAAUUGGCAACGAAUAAGCGAGGACUGUA UCAGGCAAAAGGACAGAGGA (SEQ ID NO:2 (VC I)), (linker) and CCUCUGGAGAGAACCGU UUAAUCGGUCGCCGAAGGAGCAAGUCUGCGCAUAUGCAGAGUGAAACUC UCAGGCAAAAGGACAGAGGA (SEQ ID NO:3 (VC II)).

FIGS. 2A-2F show the distribution and alignment of glycine-responsive riboswitch sequences in a variety of organisms.

FIGS. 3A-3C show the structure and in-line probing of VC II RNA of a glycine responsive riboswitch. The sequence in FIG. 3A is SEQ ID NO:4.

FIGS. 4A-4B ligand specificity of a glycine-responsive riboswitch.

FIG. 5 shows cooperative binding of two glycine molecules by VC I-II RNA of a glycine responsive riboswitch.

FIGS. 6A-6B show expected and measured response to ligand binding with RNA constructs carrying one aptamer or two aptamers of a glycine responsive riboswitch.

FIGS. 7A-7C show cooperative binding between the type I and type II aptamers of the Vibrio cholerae glycine-responsive riboswitch. The sequence in FIG. 7A is SEQ ID NO:5.

FIGS. 8A-8C show the structure and properties of a glycine-responsive riboswitch from Bacillus subtilis.

FIGS. 9A-9C show in vitro transcription of the Bacillus subtilis glycine-responsive riboswitch in the presence of various compounds. The sequences in FIG. 9A are SEQ ID NO:6 (I) and SEQ ID NO:7 (II).

FIGS. 10A-10B show the effect of glycine and glycine analogs on a glycine-responsive riboswitch.

### DETAILED DESCRIPTION OF THE INVENTION

The disclosed methods and compositions can be understood more readily by reference to the following detailed description of particular embodiments and the Example included therein and to the Figures and their previous and following description.

Certain natural mRNAs serve as metabolite-sensitive genetic switches wherein the RNA directly binds a small organic molecule. This binding process changes the conformation of the mRNA, which causes a change in gene expression by a variety of different mechanisms. Modified versions of these natural “riboswitches” (created by using various nucleic acid engineering strategies) can be employed as designer genetic switches that are controlled by specific effector compounds (referred to herein as trigger molecules). The natural switches are targets for antibiotics and other small molecule therapies. In addition, the architecture of riboswitches allows actual pieces of the natural switches to be used to construct new non-immunogenic genetic control elements, for example the aptamer (molecular recognition) domain can be swapped with other non-natural aptamers (or otherwise modified) such that the new recognition domain causes genetic modulation with user-defined effector compounds. The changed switches become part of a therapy regimen—turning on, or off, or regulating protein synthesis.

Newly constructed genetic regulation networks can be applied in such areas as living biosensors, metabolic engineering of organisms, and in advanced forms of gene therapy treatments.

Messenger RNAs are typically thought of as passive carriers of genetic information that are acted upon by protein- or small RNA-regulatory factors and by ribosomes during the process of translation. It was discovered that certain mRNAs carry natural aptamer domains and that binding of specific metabolites directly to these RNA domains leads to modulation of gene expression. Natural riboswitches exhibit two surprising functions that are not typically associated with natural RNAs. First, the mRNA element can adopt distinct structural states wherein one structure serves as a precise binding pocket for its target metabolite. Second, the metabolite-induced allosteric interconversion between structural states causes a change in the level of gene expression by one of several distinct mechanisms. Riboswitches typically can be dissected into two separate domains: one that selectively binds the target (aptamer domain) and another that influences genetic control (expression platform). It is the dynamic interplay between these two domains that results in metabolite-dependent allosteric control of gene expression.

Distinct classes of riboswitches have been identified and are shown to selectively recognize activating compounds (referred to herein as trigger molecules). For example, glycine, coenzyme B₁₂, thiamine pyrophosphate (TPP), and flavin mononucleotide (FMN) activate riboswitches present in genes encoding key enzymes in metabolic or transport pathways of these compounds. The aptamer domain of each riboswitch class conforms to a highly conserved consensus sequence and structure. Thus, sequence homology searches can be used to identify related riboswitch domains. Riboswitch domains have been discovered in various organisms from bacteria, archaea, and eukarya.

One class of riboswitches that recognizes glycine has been discovered. Representative RNAs that carry the consensus sequence and structural features of guanine riboswitches are located in the 5′-untranslated region (UTR) of numerous genes of prokaryotes, where they control expression of proteins involved in glycine cleavage. The glycine-responsive riboswitch associated with the gcvT operon of Bacillus subtilis functions as a genetic ‘ON’ switch, wherein glycine binding causes a structural rearrangement that precludes formation of an intrinsic transcription terminator stem. Further, the gcvT riboswitch includes two aptamers that exhibit cooperative binding for glycine, the trigger molecule (see Examples). Glycine-sensing riboswitches are a class of RNA genetic control elements that modulate gene expression in response to changing concentrations of this compound.

Numerous other riboswitches are known that can be used together or as part of a chimeric riboswitch along with glycine-sensing riboswitches and their components.

Examples of such riboswitches and their use are described in U.S. Application Publication No. 2005-0053951, which is hereby incorporated by reference in its entirety and in particular for its description of the structure and operation of particular riboswitches.

1. General Organization of Riboswitch RNAs

Bacterial riboswitch RNAs are genetic control elements that are located primarily within the 5′-untranslated region (5′-UTR) of the main coding region of a particular mRNA. Structural probing studies reveal that riboswitch elements are generally composed of two domains: a natural aptamer (T. Hermann, D. J. Patel, Science 2000, 287, 820; L. Gold, et al., Annual Review of Biochemistry 1995, 64, 763) that serves as the ligand-binding domain, and an ‘expression platform’ that interfaces with RNA elements that are involved in gene expression (e.g. Shine-Dalgarno (SD) elements; transcription terminator stems). These conclusions are drawn from the observation that aptamer domains synthesized in vitro bind the appropriate ligand in the absence of the expression platform (see Examples 2, 3 and 6 of U.S. Application Publication No, 2005-0053951). Moreover, structural probing investigations suggest that the aptamer domain of most riboswitches adopts a particular secondary- and tertiary-structure fold when examined independently, that is essentially identical to the aptamer structure when examined in the context of the entire 5′ leader RNA. This implies that, in many cases, the aptamer domain is a modular unit that folds independently of the expression platform (see Examples 2, 3 and 6 of U.S. Application Publication No. 2005-0053951).

Ultimately, the ligand-bound or unbound status of the aptamer domain is interpreted through the expression platform, which is responsible for exerting an influence upon gene expression. The view of a riboswitch as a modular element is further supported by the fact that aptamer domains are highly conserved amongst various organisms (and even between kingdoms as is observed for the TPP riboswitch (Sudarsan, et al., RNA 2003, 9, 644)), whereas the expression platform varies in sequence, structure, and in the mechanism by which expression of the appended open reading frame is controlled. For example, ligand binding to the TPP riboswitch of the tenA mRNA of B. subtilis causes transcription termination (Mironov et al., Cell 2002, 111, 747). This expression platform is distinct in sequence and structure compared to the expression platform of the TPP riboswitch in the thiM RNA from E. coli, wherein TPP binding causes inhibition of translation by a SD blocking mechanism (see Example 2 of U.S. Application Publication No. 2005-0053951). The TPP aptamer domain is easily recognizable and of near identical functional character between these two transcriptional units, but the genetic control mechanisms and the expression platforms that carry them out are very different.

Aptamer domains for riboswitch RNAs typically range from ˜70 to 170 nt in length (FIG. 11 of U.S. Application Publication No. 2005-0053951). This observation was somewhat unexpected given that in vitro evolution experiments identified a wide variety of small molecule-binding aptamers, which are considerably shorter in length and structural intricacy (Hermann and Patel, Science 2000, 287, 820; Gold et al., Annual Review of Biochemistry 1995, 64, 763; Famulok, Current Opinion in Structural Biology 1999, 9, 324). Although the reasons for the substantial increase in complexity and information content of the natural aptamer sequences relative to artificial aptamers remains to be proven, this complexity is most likely required to form RNA receptors that function with high affinity and selectivity. Apparent K_{D} values for the ligand-riboswitch complexes range from low nanomolar to low micromolar. It is also worth noting that some aptamer domains, when isolated from the appended expression platform, exhibit improved affinity for the target ligand over that of the intact riboswitch (˜10 to 100-fold; see Example 2 of U.S. Application Publication No. 2005-0053951). Presumably, there is an energetic cost in sampling the multiple distinct RNA conformations required by a fully intact riboswitch RNA, which is reflected by a loss in ligand affinity. Since the aptamer domain must serve as a molecular switch, this might also add to the functional demands on natural aptamers that might help rationalize their more sophisticated structures.

2. Riboswitch Regulation of Transcription Termination in Bacteria

Bacteria primarily make use of two methods for termination of transcription. Certain genes incorporate a termination signal that is dependent upon the Rho protein, (Richardson, Biochimica et Biophysica Acta 2002, 1577, 251) while others make use of Rho-independent terminators (intrinsic terminators) to destabilize the transcription elongation complex (Gusarov and Nudler, Molecular Cell 1999, 3, 495; Nudler and Gottesman, Genes to Cells 2002, 7, 755). The latter RNA elements are composed of a CC-rich stem-loop followed by a stretch of 6-9 uridyl residues. Intrinsic terminators are widespread throughout bacterial genomes (Lillo et al., 2002, 18, 971), and are typically located at the 3′-termini of genes or operons. Interestingly, an increasing number of examples are being observed for intrinsic terminators located within 5′-UTRs.

Amongst the wide variety of genetic regulatory strategies employed by bacteria there is a growing class of examples wherein RNA polymerase responds to a termination signal within the 5′-UTR in a regulated fashion (Henkin, Current Opinion in Microbiology 2000, 3, 149). During certain conditions the RNA polymerase complex is directed by external signals either to perceive or to ignore the termination signal. Although transcription initiation might occur without regulation, control over mRNA synthesis (and of gene expression) is ultimately dictated by regulation of the intrinsic terminator. Presumably, one of at least two mutually exclusive mRNA conformations results in the formation or disruption of the RNA structure that signals transcription termination. A trans-acting factor, which in some instances is a RNA (Grundy et al., Proceedings of the National Academy of Sciences of the United States of America 2002, 99, 11121; T. M. Henkin, C. Yanofsky, Bioessays 2002, 24, 700) and in others is a protein (Stulke, Archives of Microbiology 2002, 177, 433), is generally required for receiving a particular intracellular signal and subsequently stabilizing one of the RNA conformations. Riboswitches offer a direct link between RNA structure modulation and the metabolite signals that are interpreted by the genetic control machinery. A brief overview of the FMN riboswitch from a B. subtilis mRNA is provided below to illustrate this mechanism.

It was discovered that certain mRNAs involved in thiamine biosynthesis bind to thiamine (vitamin B₁) or its bioactive pyrophosphate derivative (TPP) without the participation of protein factors. The mRNA-effector complex adopts a distinct structure that sequesters the ribosome-binding site and leads to a reduction in gene expression. This metabolite-sensing mRNA system provides an example of a genetic “riboswitch” (referred to herein as a riboswitch) whose origin might predate the evolutionary emergence of proteins. It has been discovered that the mRNA leader sequence of the btuB gene of Escherichia coli can bind coenzyme B₁₂ selectively, and that this binding event brings about a structural change in the RNA that is important for genetic control (see Example 1 of U.S. Application Publication No. 2005-0053951). It was also discovered that mRNAs that encode thiamine biosynthetic proteins also employ a riboswitch mechanism (see Example 2 of U.S. Application Publication No. 2005-0053951).

A previously unknown riboswitch class was discovered in bacteria that is selectively triggered by glycine. A representative of these glycine-sensing RNAs from Bacillus subtilis operates as a rare genetic on switch for the gcvT operon, which codes for proteins that form the glycine cleavage system. Most glycine riboswitches integrate two ligand-binding domains that function cooperatively to more closely approximate a two-state genetic switch. This advanced form of riboswitch may have evolved to ensure that excess glycine is efficiently used to provide carbon flux through the citric acid cycle and maintain adequate amounts of the amino acid for protein synthesis. Thus, riboswitches perform key regulatory roles and exhibit complex performance characteristics that previously had been observed only with protein factors.

Although the specific natural riboswitches disclosed herein are the first examples of mRNA elements that control genetic expression by metabolite binding, it is expected that this genetic control strategy is widespread in biology. It has been suggested (White III, Coenzymes as fossils of an earlier metabolic state. J. Mol. Evol. 7, 101-104 (1976); White III, In: The Pyridine Nucleotide Coenzymes. Acad. Press, NY pp. 1-17 (1982); Benner et al., Modern metabolism as a palimpsest of the RNA world. Proc. Natl. Acad. Sci. USA 86, 7054-7058 (1989)) that TPP, coenzyme B₁₂ and FMN emerged as biological cofactors during the RNA world (Joyce, The antiquity of RNA-based evolution. Nature 418, 214-221 (2002)). If these metabolites were being biosynthesized and used before the advent of proteins, then certain riboswitches might be modern examples of the most ancient form of genetic control. A search of genomic sequence databases has revealed that sequences corresponding to the TPP aptamer exist in organisms from bacteria, archaea and eukarya-largely without major alteration. Although new metabolite-binding mRNAs are likely to emerge as evolution progresses, it is possible that the known riboswitches are molecular fossils from the RNA world.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference to each of various individual and collective combinations and permutation of these compounds can not be explicitly disclosed, each is specifically contemplated and described herein. For example, if a riboswitch or aptamer domain is disclosed and discussed and a number of modifications that can be made to a number of molecules including the riboswitch or aptamer domain are discussed, each and every combination and permutation of riboswitch or aptamer domain and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Riboswitches

Riboswitches are expression control elements that are part of an RNA molecule to be expressed and that change state when bound by a trigger molecule. Riboswitches typically can be dissected into two separate domains: one that selectively binds the target (aptamer domain) and another that influences genetic control (expression platform domain). It is the dynamic interplay between these two domains that results in metabolite-dependent allosteric control of gene expression. Disclosed are isolated and recombinant riboswitches, recombinant constructs containing such riboswitches, heterologous sequences operably linked to such riboswitches, and cells and transgenic organisms harboring such riboswitches, riboswitch recombinant constructs, and riboswitches operably linked to heterologous sequences. The heterologous sequences can be, for example, sequences encoding proteins or peptides of interest, including reporter proteins or peptides. Preferred riboswitches are, or are derived from, naturally occurring riboswitches.

The disclosed riboswitches, including the derivatives and recombinant forms thereof, generally can be from any source, including naturally occurring riboswitches and riboswitches designed de novo. Any such riboswitches can be used in or with the disclosed methods. However, different types of riboswitches can be defined and some such sub-types can be useful in or with particular methods (generally as described elsewhere herein). Types of riboswitches include, for example, naturally occurring riboswitches, derivatives and modified forms of naturally occurring riboswitches, chimeric riboswitches, and recombinant riboswitches. A naturally occurring riboswitch is a riboswitch having the sequence of a riboswitch as found in nature. Such a naturally occurring riboswitch can be an isolated or recombinant form of the naturally occurring riboswitch as it occurs in nature. That is, the riboswitch has the same primary structure but has been isolated or engineered in a new genetic or nucleic acid context. Chimeric riboswitches can be made up of, for example, part of a riboswitch of any or of a particular class or type of riboswitch and part of a different riboswitch of the same or of any different class or type of riboswitch; part of a riboswitch of any or of a particular class or type of riboswitch and any non-riboswitch sequence or component. Recombinant riboswitches are riboswitches that have been isolated or engineered in a new genetic or nucleic acid context.

Riboswitches can have single or multiple aptamer domains. Aptamer domains in riboswitches having multiple aptamer domains can exhibit cooperative binding of trigger molecules or can not exhibit cooperative binding of trigger molecules (that is, the aptamers need not exhibit cooperative binding). In the latter case, the aptamer domains can be said to be independent binders. Riboswitches having multiple aptamers can have one or multiple expression platform domains. For example, a riboswitch having two aptamer domains that exhibit cooperative binding of their trigger molecules can be linked to a single expression platform domain that is regulated by both aptamer domains. Riboswitches having multiple aptamers can have one or more of the aptamers joined via a linker. Where such aptamers exhibit cooperative binding of trigger molecules, the linker can be a cooperative linker.

Aptamer domains can be said to exhibit cooperative binding if they have a Hill coefficient n between x and x-1, where x is the number of aptamer domains (or the number of binding sites on the aptamer domains) that are being analyzed for cooperative binding. Thus, for example, a riboswitch having two aptamer domains (such as glycine-responsive riboswitches) can be said to exhibit cooperative binding if the riboswitch has Hill coefficient between 2 and 1. It should be understood that the value of x used depends on the number of aptamer domains being analyzed for cooperative binding, not necessarily the number of aptamer domains present in the riboswitch. This makes sense because a riboswitch may have multiple aptamer domains where only some exhibit cooperative binding.

Different classes of riboswitches refer to riboswitches that have the same or similar trigger molecules or riboswitches that have the same or similar overall structure (predicted, determined, or a combination). Riboswitches of the same class generally, but need not, have both the same or similar trigger molecules and the same or similar overall structure. Riboswitch classes include glycine-responsive riboswitches, guanine-responsive riboswitch, adenine-responsive riboswitch, lysine-responsive riboswitch, thiamine pyrophosphate-responsive riboswitch, adenosylcobalamin-responsive riboswitch, flavin mononucleotide-responsive riboswitch, and a S-adenosylmethionine-responsive riboswitch.

Also disclosed are chimeric riboswitches containing heterologous aptamer domains and expression platform domains. That is, chimeric riboswitches are made up an aptamer domain from one source and an expression platform domain from another source. The heterologous sources can be from, for example, different specific riboswitches, different types of riboswitches, or different classes of riboswitches. The heterologous aptamers can also come from non-riboswitch aptamers. The heterologous expression platform domains can also come from non-riboswitch sources.

Riboswitches can be modified from other known, developed or naturally-occurring riboswitches. For example, switch domain portions can be modified by changing one or more nucleotides while preserving the known or predicted secondary, tertiary, or both secondary and tertiary structure of the riboswitch. For example, both nucleotides in a base pair can be changed to nucleotides that can also base pair. Changes that allow retention of base pairing are referred to herein as base pair conservative changes.

Modified or derivative riboswitches can also be produced using in vitro selection and evolution techniques. In general, in vitro evolution techniques as applied to riboswitches involve producing a set of variant riboswitches where part(s) of the riboswitch sequence is varied while other parts of the riboswitch are held constant. Activation, deactivation or blocking (or other functional or structural criteria) of the set of variant riboswitches can then be assessed and those variant riboswitches meeting the criteria of interest are selected for use or further rounds of evolution. Useful base riboswitches for generation of variants are the specific and consensus riboswitches disclosed herein. Consensus riboswitches can be used to inform which part(s) of a riboswitch to vary for in vitro selection and evolution.

Also disclosed are modified riboswitches with altered regulation. The regulation of a riboswitch can be altered by operably linking an aptamer domain to the expression platform domain of the riboswitch (which is a chimeric riboswitch). The aptamer domain can then mediate regulation of the riboswitch through the action of, for example, a trigger molecule for the aptamer domain. Aptamer domains can be operably linked to expression platform domains of riboswitches in any suitable manner, including, for example, by replacing the normal or natural aptamer domain of the riboswitch with the new aptamer domain. Generally, any compound or condition that can activate, deactivate or block the riboswitch from which the aptamer domain is derived can be used to activate, deactivate or block the chimeric riboswitch.

Also disclosed are inactivated riboswitches. Riboswitches can be inactivated by covalently altering the riboswitch (by, for example, crosslinking parts of the riboswitch or coupling a compound to the riboswitch). Inactivation of a riboswitch in this manner can result from, for example, an alteration that prevents the trigger molecule for the riboswitch from binding, that prevents the change in state of the riboswitch upon binding of the trigger molecule, or that prevents the expression platform domain of the riboswitch from affecting expression upon binding of the trigger molecule.

Also disclosed are biosensor riboswitches. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a riboswitch that includes a conformation dependent label, the signal from which changes depending on the activation state of the riboswitch. Such a biosensor riboswitch preferably uses an aptamer domain from or derived from a naturally occurring riboswitch. Biosensor riboswitches can be used in various situations and platforms. For example, biosensor riboswitches can be used with solid supports, such as plates, chips, strips and wells.

Also disclosed are modified or derivative riboswitches that recognize new trigger molecules. New riboswitches and/or new aptamers that recognize new trigger molecules can be selected for, designed or derived from known riboswitches. This can be accomplished by, for example, producing a set of aptamer variants in a riboswitch, assessing the activation of the variant riboswitches in the presence of a compound of interest, selecting variant riboswitches that were activated (or, for example, the riboswitches that were the most highly or the most selectively activated), and repeating these steps until a variant riboswitch of a desired activity, specificity, combination of activity and specificity, or other combination of properties results.

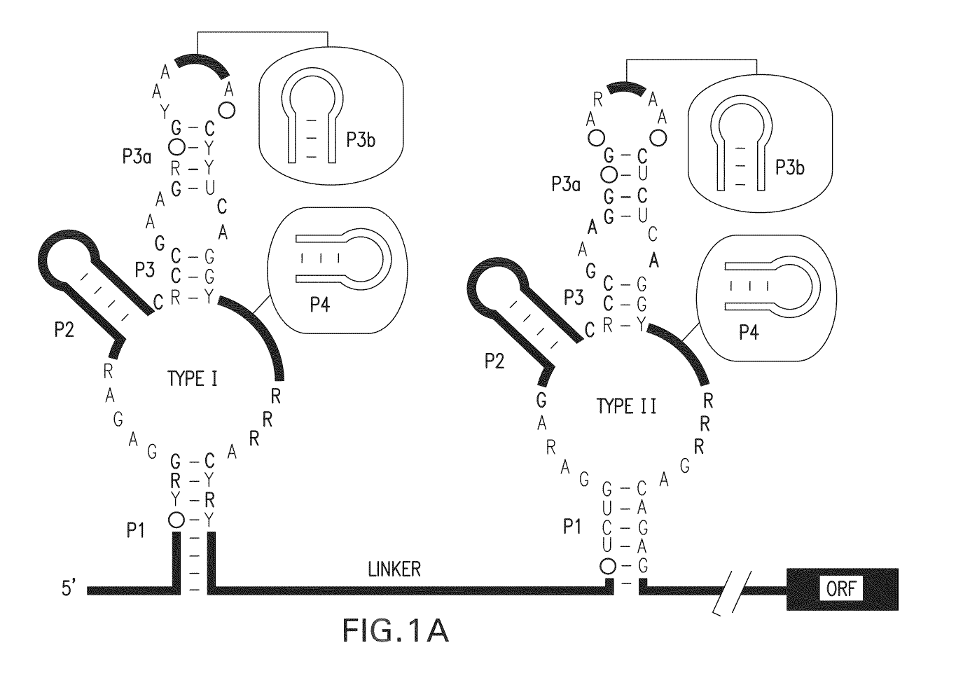

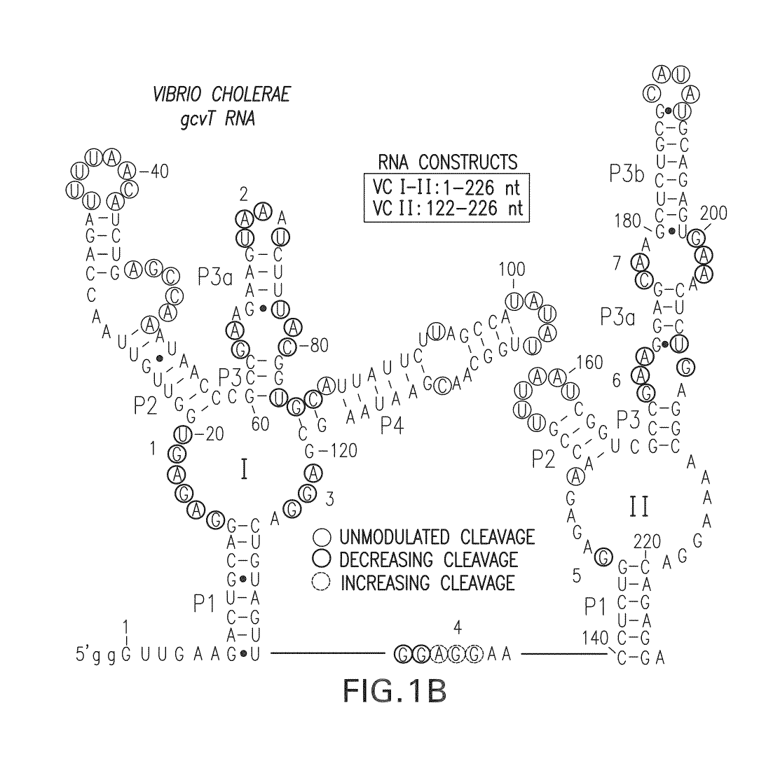

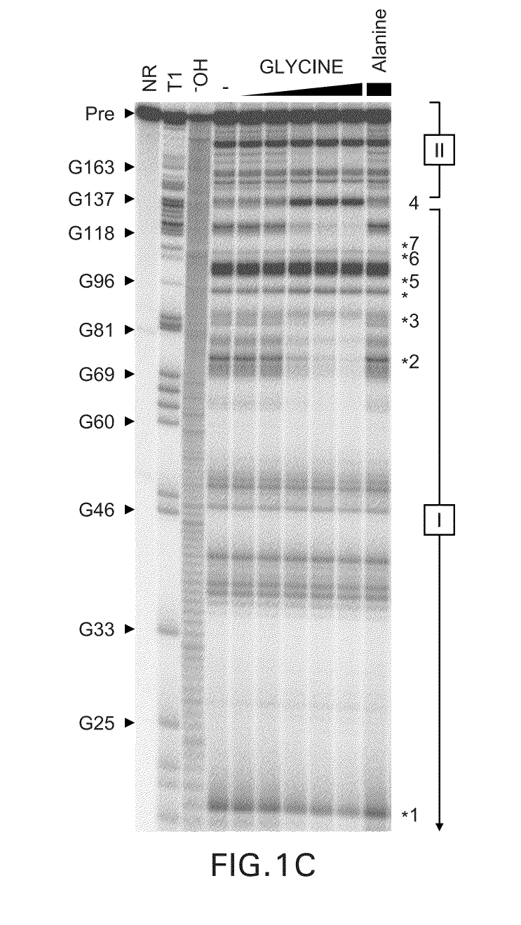

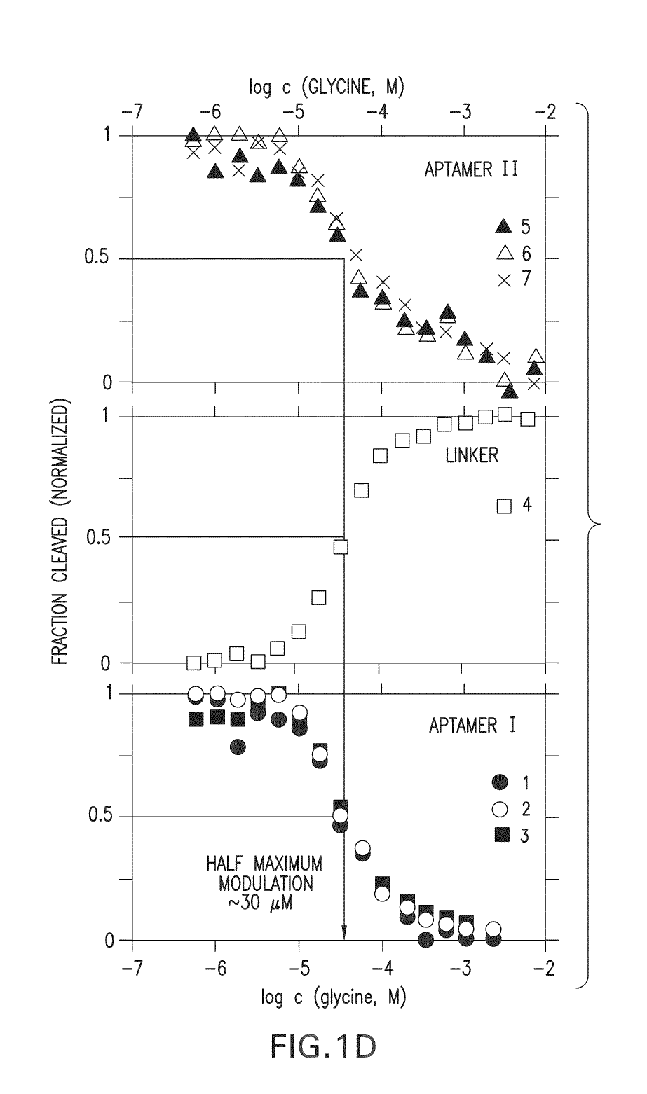

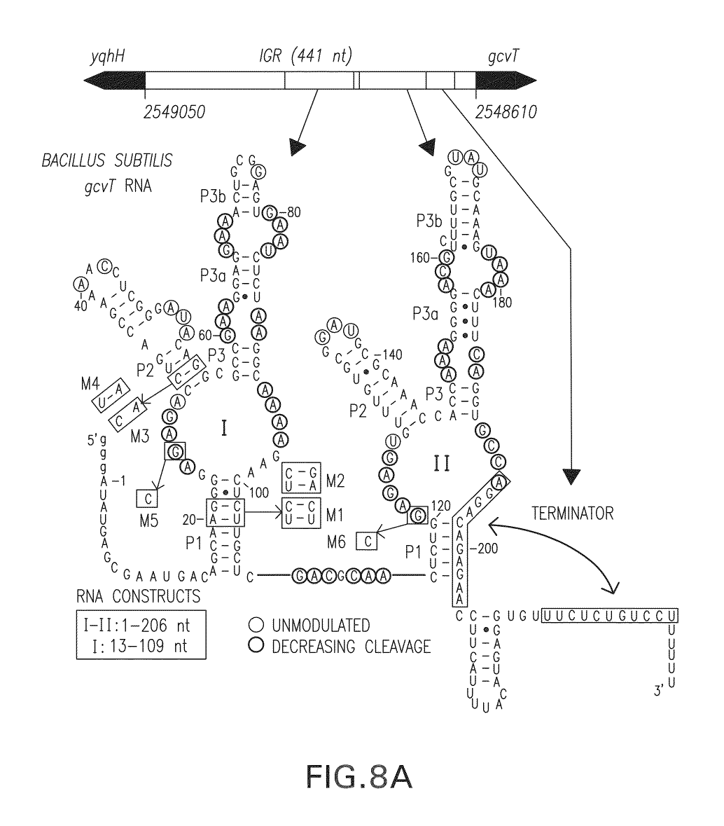

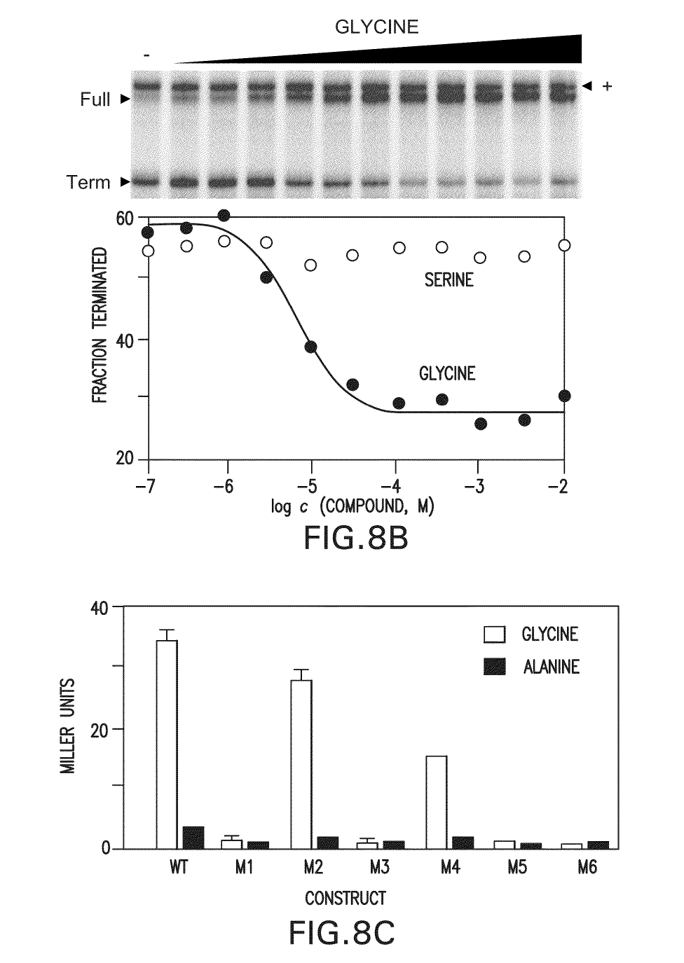

Particularly useful aptamer domains can form a stem structure referred to herein as the P1 stem structure (or simply P1). The P1 stems of a variety of riboswitches are shown in FIG. 11 of U.S. Application Publication No. 2005-0053951. FIGS. 1 and 8 show P1 stems of glycine-responsive riboswitches. The hybridizing strands in the P1 stem structure are referred to as the aptamer strand (also referred to as the P1a strand) and the control strand (also referred to as the P1b strand). The control strand can form a stem structure with both the aptamer strand and a sequence in a linked expression platform that is referred to as the regulated strand (also referred to as the P1c strand). Thus, the control strand (P1b) can form alternative stem structures with the aptamer strand (P1a) and the regulated strand (P1c). Activation and deactivation of a riboswitch results in a shift from one of the stem structures to the other (from P1a/P1b to P1b/P1c or vice versa). The formation of the P1b/P1c stem structure affects expression of the RNA molecule containing the riboswitch. Riboswitches that operate via this control mechanism are referred to herein as alternative stem structure riboswitches (or as alternative stem riboswitches). Some glycine-responsive riboswitches having two aptamers utilize this mechanism using a P1 stem in the second aptamer (see FIGS. 1 and 8).

In general, any aptamer domain can be adapted for use with any expression platform domain by designing or adapting a regulated strand in the expression platform domain to be complementary to the control strand of the aptamer domain. Alternatively, the sequence of the aptamer and control strands of an aptamer domain can be adapted so that the control strand is complementary to a functionally significant sequence in an expression platform. For example, the control strand can be adapted to be complementary to the Shine-Dalgarno sequence of an RNA such that, upon formation of a stem structure between the control strand and the SD sequence, the SD sequence becomes inaccessible to ribosomes, thus reducing or preventing translation initiation. Note that the aptamer strand would have corresponding changes in sequence to allow formation of a P1 stem in the aptamer domain. In the case of riboswitches having multiple aptamers exhibiting cooperative binding, one the P1 stem of the activating aptamer (the aptamer that interacts with the expression platform domain) need be designed to form a stem structure with the SD sequence.

As another example, a transcription terminator can be added to an RNA molecule (most conveniently in an untranslated region of the RNA) where part of the sequence of the transcription terminator is complementary to the control strand of an aptamer domain (the sequence will be the regulated strand). This will allow the control sequence of the aptamer domain to form alternative stem structures with the aptamer strand and the regulated strand, thus either forming or disrupting a transcription terminator stem upon activation or deactivation of the riboswitch. Any other expression element can be brought under the control of a riboswitch by similar design of alternative stem structures.

For transcription terminators controlled by riboswitches, the speed of transcription and spacing of the riboswitch and expression platform elements can be important for proper control. Transcription speed can be adjusted by, for example, including polymerase pausing elements (e.g., a series of uridine residues) to pause transcription and allow the riboswitch to form and sense trigger molecules. For example, with the FMN riboswitch, if FMN is bound to its aptamer domain, then the antiterminator sequence is sequestered and is unavailable for formation of an antiterminator structure (FIG. 12 of U.S. Application Publication No. 2005-0053951). However, if FMN is absent, the antiterminator can form once its nucleotides emerge from the polymerase. RNAP then breaks free of the pause site only to reach another U-stretch and pause again. The transcriptional terminator then forms only if the terminator nucleotides are not tied up by the antiterminator.

Disclosed are regulatable gene expression constructs comprising a nucleic acid molecule encoding an RNA comprising a riboswitch operably linked to a coding region, wherein the riboswitch regulates expression of the RNA, wherein the riboswitch and coding region are heterologous. The riboswitch can comprise an aptamer domain and an expression platform domain, wherein the aptamer domain and the expression platform domain are heterologous. The riboswitch can comprise an aptamer domain and an expression platform domain, wherein the aptamer domain comprises a P1 stem, wherein the P1 stem comprises an aptamer strand and a control strand, wherein the expression platform domain comprises a regulated strand, wherein the regulated strand, the control strand, or both have been designed to form a stem structure. The riboswitch can comprise two or more aptamer domains and an expression platform domain, wherein at least one of the aptamer domains and the expression platform domain are heterologous. The riboswitch can comprise two or more aptamer domains and an expression platform domain, wherein at least one of the aptamer domains comprises a P1 stem, wherein the P1 stem comprises an aptamer strand and a control strand, wherein the expression platform domain comprises a regulated strand, wherein the regulated strand, the control strand, or both have been designed to form a stem structure.

Disclosed are riboswitches, wherein the riboswitch is a non-natural derivative of a naturally-occurring riboswitch. The riboswitch can comprise an aptamer domain and an expression platform domain, wherein the aptamer domain and the expression platform domain are heterologous. The riboswitch can be derived from a naturally-occurring glycine-responsive riboswitch, guanine-responsive riboswitch, adenine-responsive riboswitch, lysine-responsive riboswitch, thiamine pyrophosphate-responsive riboswitch, adenosylcobalamin-responsive riboswitch, flavin mononucleotide-responsive riboswitch, or a S-adenosylmethionine-responsive riboswitch. The riboswitch can be activated by a trigger molecule, wherein the riboswitch produces a signal when activated by the trigger molecule.

Numerous riboswitches and riboswitch constructs are described and referred to herein. It is specifically contemplated that any specific riboswitch or riboswitch construct or group of riboswitches or riboswitch constructs can be excluded from some aspects of the invention disclosed herein. For example, fusion of the xpt-pbuX riboswitch with a reporter gene could be excluded from a set of riboswitches fused to reporter genes.

1. Aptamer Domains

Aptamers are nucleic acid segments and structures that can bind selectively to particular compounds and classes of compounds. Riboswitches have aptamer domains that, upon binding of a trigger molecule result in a change the state or structure of the riboswitch. In functional riboswitches, the state or structure of the expression platform domain linked to the aptamer domain changes when the trigger molecule binds to the aptamer domain. Aptamer domains of riboswitches can be derived from any source, including, for example, natural aptamer domains of riboswitches, artificial aptamers, engineered, selected, evolved or derived aptamers or aptamer domains. Aptamers in riboswitches generally have at least one portion that can interact, such as by forming a stem structure, with a portion of the linked expression platform domain. This stem structure will either form or be disrupted upon binding of the trigger molecule.

Consensus aptamer domains of a variety of natural riboswitches are shown in FIG. 1 herein and in FIG. 11 of U.S. Application Publication No. 2005-0053951. These aptamer domains (including all of the direct variants embodied therein) can be used in riboswitches. The consensus sequences and structures indicate variations in sequence and structure. Aptamer domains that are within the indicated variations are referred to herein as direct variants. These aptamer domains can be modified to produce modified or variant aptamer domains. Conservative modifications include any change in base paired nucleotides such that the nucleotides in the pair remain complementary. Moderate modifications include changes in the length of stems or of loops (for which a length or length range is indicated) of less than or equal to 20% of the length range indicated. Loop and stem lengths are considered to be “indicated” where the consensus structure shows a stem or loop of a particular length or where a range of lengths is listed or depicted. Moderate modifications include changes in the length of stems or of loops (for which a length or length range is not indicated) of less than or equal to 40% of the length range indicated. Moderate modifications also include and functional variants of unspecified portions of the aptamer domain. Unspecified portions of the aptamer domains are indicated by solid lines in FIG. 1 herein and in FIG. 11 of U.S. Application Publication No. 2005-0053951.

The P1 stem and its constituent strands can be modified in adapting aptamer domains for use with expression platforms and RNA molecules. Such modifications, which can be extensive, are referred to herein as P1 modifications. P1 modifications include changes to the sequence and/or length of the P1 stem of an aptamer domain.

The aptamer domains shown in FIG. 1 and in FIG. 11 of U.S. Application Publication No. 2005-0053951 (including any direct variants) are particularly useful as initial sequences for producing derived aptamer domains via in vitro selection or in vitro evolution techniques.

Aptamer domains of the disclosed riboswitches can also be used for any other purpose, and in any other context, as aptamers. For example, aptamers can be used to control ribozymes, other molecular switches, and any RNA molecule where a change in structure can affect function of the RNA.

2. Expression Platform Domains

Expression platform domains are a part of riboswitches that affect expression of the RNA molecule that contains the riboswitch. Expression platform domains generally have at least one portion that can interact, such as by forming a stem structure, with a portion of the linked aptamer domain. This stem structure will either form or be disrupted upon binding of the trigger molecule. The stem structure generally either is, or prevents formation of, an expression regulatory structure. An expression regulatory structure is a structure that allows, prevents, enhances or inhibits expression of an RNA molecule containing the structure. Examples include Shine-Dalgarno sequences, initiation codons, transcription terminators, and stability and processing signals.

### B. Trigger Molecules

Trigger molecules are molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques). Non-natural trigger molecules can be referred to as non-natural trigger molecules.

### C. Compounds

Also disclosed are compounds, and compositions containing such compounds, that can activate, deactivate or block a riboswitch. Riboswitches function to control gene expression through the binding or removal of a trigger molecule. Compounds can be used to activate, deactivate or block a riboswitch. The trigger molecule for a riboswitch (as well as other activating compounds) can be used to activate a riboswitch. Compounds other than the trigger molecule generally can be used to deactivate or block a riboswitch. Riboswitches can also be deactivated by, for example, removing trigger molecules from the presence of the riboswitch. A riboswitch can be blocked by, for example, binding of an analog of the trigger molecule that does not activate the riboswitch.

Also disclosed are compounds for altering expression of an mRNA molecule, or of a gene encoding an RNA molecule, where the RNA molecule includes a riboswitch. This can be accomplished by bringing a compound into contact with the RNA molecule. Riboswitches function to control gene expression through the binding or removal of a trigger molecule. Thus, subjecting an RNA molecule of interest that includes a riboswitch to conditions that activate, deactivate or block the riboswitch can be used to alter expression of the RNA. Expression can be altered as a result of, for example, termination of transcription or blocking of ribosome binding to the RNA. Binding of a trigger molecule can, depending on the nature of the riboswitch, reduce or prevent expression of the RNA molecule or promote or increase expression of the RNA molecule.

Also disclosed are compounds for regulating expression of an RNA molecule, or of a gene encoding an RNA molecule. Also disclosed are compounds for regulating expression of a naturally occurring gene or RNA that contains a riboswitch by activating, deactivating or blocking the riboswitch. If the gene is essential for survival of a cell or organism that harbors it, activating, deactivating or blocking the riboswitch can in death, stasis or debilitation of the cell or organism.

Also disclosed are compounds for regulating expression of an isolated, engineered or recombinant gene or RNA that contains a riboswitch by activating, deactivating or blocking the riboswitch. If the gene encodes a desired expression product, activating or deactivating the riboswitch can be used to induce expression of the gene and thus result in production of the expression product. If the gene encodes an inducer or repressor of gene expression or of another cellular process, activation, deactivation or blocking of the riboswitch can result in induction, repression, or de-repression of other, regulated genes or cellular processes. Many such secondary regulatory effects are known and can be adapted for use with riboswitches. An advantage of riboswitches as the primary control for such regulation is that riboswitch trigger molecules can be small, non-antigenic molecules.

Also disclosed are methods of identifying compounds that activate, deactivate or block a riboswitch. For examples, compounds that activate a riboswitch can be identified by bringing into contact a test compound and a riboswitch and assessing activation of the riboswitch. If the riboswitch is activated, the test compound is identified as a compound that activates the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

Identification of compounds that block a riboswitch can be accomplished in any suitable manner. For example, an assay can be performed for assessing activation or deactivation of a riboswitch in the presence of a compound known to activate or deactivate the riboswitch and in the presence of a test compound. If activation or deactivation is not observed as would be observed in the absence of the test compound, then the test compound is identified as a compound that blocks activation or deactivation of the riboswitch.

Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound.

This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

1. Chemical Definitions Section

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“A¹,” “A²,” “A,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 40 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “halogenated alkyl” is used in another, it is not meant to imply that the term “alkyl” does not also refer to specific terms such as “halogenated alkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bonded through a single, terminal ether linkage; that is, an “alkoxy” group can be defined as —OA¹ where A² is alkyl as defined above. Polymers of alkoxy groups are referred to herein as “polyethers” such as —OA¹-OA² or —OA¹-(OA²)ₐ-OA³, where “a” is some integer and A¹, A², and A³ are alkyl groups.

The term “alkenyl” as used herein is a hydrocarbon group of from 2 to 40 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol, as described below.

The term “alkynyl” as used herein is a hydrocarbon group of 2 to 40 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” is a short hand notation for C═O.

The terms “amine” or “amino” as used herein are represented by the formula NA¹A²A³, where A¹, A², and A can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O—.

The term “ester” as used herein is represented by the formula —OC(O)A¹ or —C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above.

The term “polyester” as used herein is represented by the formula -(A¹OC(O)A²OC(O))ₐ—, where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described herein and “a” is some integer. “Polyester” is also the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

The term “sulfo-oxo” as used herein is represented by the formulas —S(O)A¹ (i.e., “sulfonyl”), A¹S(O)A² (i.e., “sulfoxide”), —S(O)₂A¹, A¹SO₂A (i.e., “sulfone”), —OS(O)₂A¹, or —OS(O)₂OA¹, where A¹ and A² can be hydrogen, an alkyl, halogenated alkyl, alkenyl, alkynyl, aryl, heteroaryl, cycloalkyl, cycloalkenyl, heterocycloalkyl, or heterocycloalkenyl group described above. Throughout this specification “S(O)” is a short hand notation for S═O.

The term “sulfonylamino” or “sulfonamide” as used herein is represented by the formula —S(O)₂ NH—.

The term “thiol” as used herein is represented by the formula —SH.

“L,” “X,” “R,” as used herein can, independently, possess one or more of the groups listed above. For example, if L is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

2. Materials and Compositions

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers inc., 1989).

In one aspect disclosed herein are compositions having a glycine residue bonded to a linker having one or more moieties, where the composition is capable of binding to a riboswitch. The disclosed compounds can be represented by Formula I:

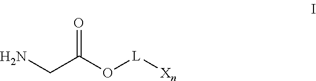

where L is a linker, X is a moiety, and n is an integer from 1 to 10. It can be desirable that the disclosed compounds be bioavailable, bind a riboswitch tightly, be non-toxic to a subject, and have desirable pharmacokinetic properties. Such compounds are useful with guanine-responsive riboswitches (and riboswitches derived from guanine-responsive riboswitches).

Every compound within the above definition is intended to be and should be considered to be specifically disclosed herein. Further, every subgroup that can be identified within the above definition is intended to be and should be considered to be specifically disclosed herein. As a result, it is specifically contemplated that any compound, or subgroup of compounds can be either specifically included for or excluded from use or included in or excluded from a list of compounds. For example, as one option, a group of compounds is contemplated where each compound is as defined above but is not glycine. As another example, a group of compounds is contemplated where each compound is as defined above and is able to activate a glycine-responsive riboswitch.

i. Linker (L)

The linker moiety of the disclosed compositions (L) can be any moiety that can connect the glycine residue to one or more moieties (X). As disclosed herein, the moiety (X) can be originally present on the linker, derived from functional groups present on the linker through a functional group transformation, or bonded to the linking moiety prior to, during, or after the linking moiety is coupled to the glycine residue. The attachment of the linker (L) to the glycine residue and/or moiety can be via a covalent bond by reaction methods known in the art. For example, the moiety (X) can be already present on the linker or first coupled to the linker, and then attached to the glycine residue. Alternatively, the linker can be first coupled to the glycine residue and then attached to the moiety.

The linker can be of varying lengths, such as from 1 to 50 atoms in length. For example, the linker can be from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 atoms in length, where any of the stated values can form an upper and/or lower end point where appropriate. Further, the linker can be substituted or unsubstituted. When substituted, the linker can contain substituents attached to the backbone of the linker or substituents embedded in the backbone of the linker. For example, an amine substituted linker can contain an amine group attached to the backbone of the linker or a nitrogen in the backbone of the linker. Examples of suitable substituents include, but are not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol as described herein.

Suitable linkers include, but are not limited to, substituted or unsubstituted, branched or unbranched, alkyl, alkenyl, or alkynyl groups, ethers, esters, polyethers, polyesters, polyalkylenes, polyamines, heteroatom substituted alkyl, alkenyl, or alkynyl groups, cycloalkyl groups, cycloalkenyl groups, heterocycloalkyl groups, heterocycloalkenyl groups, and the like, and derivatives thereof.

In some examples, the linker can comprise a C₁-C₁₂ branched or straight-chain alkyl, such as methyl, ethyl, n-propyl, iso-propyl, n-butyl, iso-butyl, sec-butyl, tert-butyl, n-pentyl, iso-pentyl, neopentyl, hexyl, heptyl, octyl, nonyl, decyl, undecyl, or dodecyl group. These alkyl linkers can be unsubstituted or substituted with substituents such as, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol as described herein. In a specific example, the linker can comprise —(CH₂)ₘ—, wherein m is from 1 to 12. In other examples, the linker can comprise —(CH₂)ₘ—, wherein m is from 2, 3, 5, 6, 7, 8, 9, or 11; that is, in these examples n is not 1, 4, 10 or 12. Examples of such compounds are illustrated in Formula II, where m is an integer of from 1 to 12 and X is a moiety.

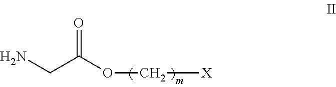

In other examples, the linker can comprise a C₂-C₁₂ branched or straight chain alkenyl or alkynyl. Such linkers can have one or more double or triple carbon-carbon bond. Such alkenyl or alkynyl linkers can be unsubstituted or substituted with substituents such as, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol as described herein.

In other examples, the linker can comprise a C₂-C₂₀ branched or straight-chain alkyl, wherein one or more carbon atoms is substituted with oxygen (e.g., an ether) or an amino group. For example, suitable linkers can include, but are not limited to, a methoxymethyl, methoxyethyl, methoxypropyl, methoxybutyl, ethoxymethyl, ethoxyethyl, ethoxypropyl, propoxymethyl, propoxyethyl, methylaminomethyl, methylaminoethyl, methylaminopropyl, methylaminobutyl, ethylaminomethyl, ethylaminoethyl, ethylaminopropyl, propylaminomethyl, propylaminoethyl, hoxymxymethoxymethyl, ethoxymethoxymethyl, methoxyethoxymethyl, methoxymethoxyethyl and the like, and derivatives thereof. Such linkers can be unsubstituted or substituted with substituents such as, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol as described herein. In one specific example, the linker can comprise a polyether, i.e., (CH₂—O—CH₂)ₘ, where m is an integer from 1 to 20. Examples of such compounds are illustrated in Formula III, where m and p are integers of from 1 to 20, Y is O or NH, and X is a moiety.

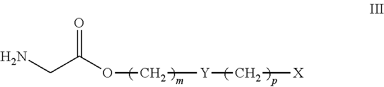

Still other examples of linkers can be polyesters. The polyester can be unsubstituted or substituted with substituents such as, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol as described herein.

Suitable linkers are readily commercially available and/or can be synthesized by those of ordinary skill in the art. And the particular linker that can be used in the disclosed composites can be chosen by one of ordinary skill in the art based on factors such as cost, convenience, availability, compatibility with various reaction conditions, the type of first and/or second active substance with which the linker is to interact, and the like.

ii. Moiety (X)

The disclosed compounds can have one or more moieties (X). Such moieties can be inert or can be reactive. For example, such moieties can be —H or not present. As another example, such moieties can be nucleophilic moieties that can react with electrophilic moieties, forming a bond. As another example, the moiety (X) can be an electrophilic moiety that can react with nucleophilic moieties, forming a bond.

By “nucleophilic moiety” is meant any moiety that contains or can be made to contain an electron rich atom; examples of nucleophilic functional groups are disclosed herein. By “electrophilic moiety” is meant any moiety that contains or can be made to contain an electron deficient atom; examples of electrophilic functional groups are also disclosed herein.

a. Nucleophilic Moieties

Examples of nucleophilic moieties include, but are not limited to, amine groups, carboxylate groups, hydroxyl groups, and thiol groups. Such nucleophilic groups can be present on the linker, described above, added to the linker, or derived from a functional group on the linker. In some examples, the nucleophilic moiety can be an amino acid residue. For example, in the disclosed compounds the moiety can be a residue of one of the twenty naturally occurring amino acids. For example, the nucleophilic or potentially nucleophilic amine present in any of the twenty amino acids can be used. Examples of such compounds are disclosed in Formula IV, where L is the linker and R is the side-chain of an amino acid (e.g., H for glycine, —C₃ for alanine, —CH(CH₃)₂ for valine, —CH₂OH for serine, and the like).

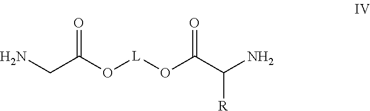

In a particular example, the functional group is another glycine residue.

It is also contemplated that in addition to or instead of the amine group, other groups on many amino acids can also be nucleophilic and thus bond to an electrophilic group. For example, carboxylate or carboxylic acid groups in the side-chain of aspartic acid or glutamic acid, hydroxyl groups in the side chain of serine, threonine, and tyrosine, the thiol group in cysteine, or the amine group of lysine can bind. Other examples of nucleophilic moieties include, but are not limited to, carbohydrates, polysaccharides, lipids, saturated and unsaturated fatty acids, or cholesterols that possess a nucleophilic or potentially nucleophilic amine, carboxylate, alcohol, or thiol functional group. These and other examples are disclosed herein.

Further, it is contemplated that more than one type of nucleophilic moiety can be present in the disclosed compounds.

b. Electrophilic Moieties

Examples of such electrophilic moieties include, but are not limited to, aldehydes, esters and activated esters (e.g., succinimidyl esters, sulfosuccinimidyl esters), derivatized carboxylic acids and carboxylates, imines, isocyanates, isothiocyanates, and maleimides. These moieties are well known in the art of organic chemistry.

Some specific examples of suitable electrophilic moieties include, but are not limited to, residues of gluteraldehyde, glyoxal, methylglyoxal, benzaldehyde, dialkyl oxalates, dialkyl fumarate, dialkyl malonate, dialkyl succinate, dialkyl adipate, dialkyl azelates, dialkyl suberate, dialkyl sebacate, dialkyl terephthalate, dialkylisophthalate, diallylphthalate, and the like.

Succinimidyl ester moieties can also react with amine, carboxylate, alcohol, or thiol functional groups. Succinimidyl esters are particularly reactive towards amines, where the resulting amide bond that is formed is as stable as a peptide bond. However, some succinimidyl ester linkers may not be compatible with a specific application because they can be quite insoluble in aqueous solution. To overcome this limitation, sulfosuccinimidyl esters, which typically have higher water solubility than succinimidyl ester linkers, can be used. Sulfosuccinimidyl esters can generally be prepared in situ from simple carboxylic acids by dissolving the acid in an amine-free buffer that contains N-hydroxyoslfosuccinimide and 1-ethyl-3-(3-dimethylaminopropyl)carbodiimide. Also, 4-sulfo-2,3,5,6-tetrafluorophenol (STP) ester can be prepared from 4-sulfo-2,3,5,6-tetrafluorophenol in the same way as sulfosuccinimidyl esters.

### D. Constructs, Vectors and Expression Systems

The disclosed riboswitches can be used in with any suitable expression system.

Recombinant expression is usefully accomplished using a vector, such as a plasmid. The vector can include a promoter operably linked to riboswitch-encoding sequence and RNA to be expression (e.g., RNA encoding a protein). The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosminds, and artificial chromosomes. A variety of prokaryotic and eukaryotic expression vectors suitable for carrying riboswitch-regulated constructs can be produced. Such expression vectors include, for example, pET, pET3d, pCR2.1, pBAD, pUC, and yeast vectors. The vectors can be used, for example, in a variety of in vivo and in vitro situation.

Viral vectors include adenovirus, adeno-associated virus, herpes virus, vaccinia virus, polio virus, AIDS virus, neuronal trophic virus, Sindbis and other RNA viruses, including these viruses with the HIV backbone. Also useful are any viral families which share the properties of these viruses which make them suitable for use as vectors. Retroviral vectors, which are described in Verma (1985), include Murine Maloney Leukemia virus, MMLV, and retroviruses that express the desirable properties of MMLV as a vector. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA.

A “promoter” is generally a sequence or sequences of DNA that function when in a relatively fixed location in regard to the transcription start site. A “promoter” contains core elements required for basic interaction of RNA polymerase and transcription factors and can contain upstream elements and response elements.

“Enhancer” generally refers to a sequence of DNA that functions at no fixed distance from the transcription start site and can be either 5′ (Laimnins, 1981) or 3′ (Lusky et al., 1983) to the transcription unit. Furthermore, enhancers can be within an intron (Banerji et al., 1983) as well as within the coding sequence itself (Osborne et al., 1984). They are usually between 10 and 300 bp in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

The vector can include nucleic acid sequence encoding a marker product. This marker product is used to determine if the gene has been delivered to the cell and once delivered is being expressed. Preferred marker genes are the E. Coli lacZ gene which encodes β-galactosidase and green fluorescent protein.

In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern and Berg, 1982), mycophenolic acid, (Mulligan and Berg, 1980) or hygromycin (Sugden et al., 1985).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

1. Viral Vectors

Preferred viral vectors are Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Preferred retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermos table and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors have higher transaction (ability to introduce genes) abilities than do most chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

i. Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

ii. Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles.

Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A preferred viral vector is one based on an adenovirus which has had the E1 gene removed and these virons are generated in a cell line such as the human 293 cell line. In another preferred embodiment both the E1 and E3 genes are removed from the adenovirus genome.

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

2. Viral Promoters and Enhancers

Preferred promoters controlling transcription from vectors in mammalian host cells can be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

It is preferred that the promoter and/or enhancer region be active in all eukaryotic cell types. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In a preferred embodiment of the transcription unit, the polyadenylation region is derived from the SV40 early polyadenylation signal and consists of about 400 bases. It is also preferred that the transcribed units contain other standard sequences alone or in combination with the above sequences improve expression from, or stability of, the construct.

3. Markers

The vectors can include nucleic acid sequence encoding a marker product. This marker product is used to determine if the gene has been delivered to the cell and once delivered is being expressed. Preferred marker genes are the E. Coli lacZ gene which encodes β-galactosidase and green fluorescent protein.

In some embodiments the marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR⁻ cells and mouse LTK⁻ cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell, Biol. 5: 410-413 (1985)). The three examples employ bacterial genes under eukaryotic control to convey resistance to the appropriate drug (G418 or neomycin (geneticin), xgpt (mycophenolic acid) or hygromycin, respectively. Others include the neomycin analog G418 and puramycin.

### E. Biosensor Riboswitches

Also disclosed are biosensor riboswitches. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a riboswitch that includes a conformation dependent label, the signal from which changes depending on the activation state of the riboswitch. Such a biosensor riboswitch preferably uses an aptamer domain from or derived from a naturally occurring riboswitch.

### F. Reporter Proteins and Peptides

For assessing activation of a riboswitch, or for biosensor riboswitches, a reporter protein or peptide can be used. The reporter protein or peptide can be encoded by the RNA the expression of which is regulated by the riboswitch. The examples describe the use of some specific reporter proteins. The use of reporter proteins and peptides is well known and can be adapted easily for use with riboswitches. The reporter proteins can be any protein or peptide that can be detected or that produces a detectable signal. Preferably, the presence of the protein or peptide can be detected using standard techniques (e.g., radioimmunoassay, radio-labeling, immunoassay, assay for enzymatic activity, absorbance, fluorescence, luminescence, and Western blot). More preferably, the level of the reporter protein is easily quantifiable using standard techniques even at low levels. Useful reporter proteins include luciferases, green fluorescent proteins and their derivatives, such as firefly luciferase (FL) from Photinus pyralis, and Renilla luciferase (RL) from Renilla reniformis.

### G. Conformation Dependent Labels

Conformation dependent labels refer to all labels that produce a change in fluorescence intensity or wavelength based on a change in the form or conformation of the molecule or compound (such as a riboswitch) with which the label is associated. Examples of conformation dependent labels used in the context of probes and primers include molecular beacons, Amplifluors, FRET probes, cleavable FRET probes, TaqMan probes, scorpion primers, fluorescent triplex oligos including but not limited to triplex molecular beacons or triplex FRET probes, fluorescent water-soluble conjugated polymers, PNA probes and QPNA probes. Such labels, and, in particular, the principles of their function, can be adapted for use with riboswitches. Several types of conformation dependent labels are reviewed in Schweitzer and Kingsmore, Curr. Opin. Biotech. 12:21-27 (2001).

Stem quenched labels, a form of conformation dependent labels, are fluorescent labels positioned on a nucleic acid such that when a stem structure forms a quenching moiety is brought into proximity such that fluorescence from the label is quenched. When the stem is disrupted (such as when a riboswitch containing the label is activated), the quenching moiety is no longer in proximity to the fluorescent label and fluorescence increases. Examples of this effect can be found in molecular beacons, fluorescent triplex oligos, triplex molecular beacons, triplex FRET probes, and QPNA probes, the operational principles of which can be adapted for use with riboswitches.

Stem activated labels, a form of conformation dependent labels, are labels or pairs of labels where fluorescence is increased or altered by formation of a stem structure. Stem activated labels can include an acceptor fluorescent label and a donor moiety such that, when the acceptor and donor are in proximity (when the nucleic acid strands containing the labels form a stem structure), fluorescence resonance energy transfer from the donor to the acceptor causes the acceptor to fluoresce. Stem activated labels are typically pairs of labels positioned on nucleic acid molecules (such as riboswitches) such that the acceptor and donor are brought into proximity when a stem structure is formed in the nucleic acid molecule. If the donor moiety of a stem activated label is itself a fluorescent label, it can release energy as fluorescence (typically at a different wavelength than the fluorescence of the acceptor) when not in proximity to an acceptor (that is, when a stem structure is not formed). When the stem structure forms, the overall effect would then be a reduction of donor fluorescence and an increase in acceptor fluorescence. FRET probes are an example of the use of stem activated labels, the operational principles of which can be adapted for use with riboswitches.

### H. Detection Labels

To aid in detection and quantitation of riboswitch activation, deactivation or blocking, or expression of nucleic acids or protein produced upon activation, deactivation or blocking of riboswitches, detection labels can be incorporated into detection probes or detection molecules or directly incorporated into expressed nucleic acids or proteins. As used herein, a detection label is any molecule that can be associated with nucleic acid or protein, directly or indirectly, and which results in a measurable, detectable signal, either directly or indirectly. Many such labels are known to those of skill in the art. Examples of detection labels suitable for use in the disclosed method are radioactive isotopes, fluorescent molecules, phosphorescent molecules, enzymes, antibodies, and ligands.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as Quantum Dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard. Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid). Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochronme Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 60, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 40, Sevron Brilliant Red B, Sevron Orange, Sevron Yellow L, SITS (Prinuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Useful fluorescent labels are fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

Labeled nucleotides are a useful form of detection label for direct incorporation into expressed nucleic acids during synthesis. Examples of detection labels that can be incorporated into nucleic acids include nucleotide analogs such as BrdUrd (5-bromodeoxyuridine, Hoy and Schimke, Mutation Research 290:217-230 (1993)), aminoallyideoxyuridine (Henegariu et al., Nature Biotechnology 18:345-348 (2000)), 5-methylcytosine (Sano et al., Biochim. Biophys. Acta 951:157-165 (1988)), bromouridine (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (bromodeoxyuridine, BrdUrd, BrdU, BUdR, Sigma-Aldrich Co). Other useful nucleotide analogs for incorporation of detection label into DNA are AA-dUTP (aminoallyl-deoxyuridine triphosphate, Sigma-Aldrich Co.), and 5-methyl-dCTP (Roche Molecular Biochemicals). A useful nucleotide analog for incorporation of detection label into RNA is biotin-16-UTP (biotin-16-uridine-5′-triphosphate, Roche Molecular Biochemicals). Fluorescein, Cy3, and Cy5 can be linked to dUTP for direct labelling. Cy3.5 and Cy7 are available as avidin or anti-digoxygenin conjugates for secondary detection of biotin- or digoxygenin-labelled probes.

Detection labels that are incorporated into nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo [3.3.1.1^{3,7}]decane]-4-v) phenyl phosphate; Tropix, Inc.). Labels can also be enzymes, such as alkaline phosphatase, soybean peroxidase, horseradish peroxidase and polymerases, that can be detected., for example, with chemical signal amplification or by using a substrate to the enzyme which produces light (for example, a chemiluminescent 1,2-dioxetane substrate) or fluorescent signal.

Molecules that combine two or more of these detection labels are also considered detection labels. Any of the known detection labels can be used with the disclosed probes, tags, molecules and methods to label and detect activated or deactivated riboswitches or nucleic acid or protein produced in the disclosed methods. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. As used herein, detection molecules are molecules which interact with a compound or composition to be detected and to which one or more detection labels are coupled.

### I. Sequence Similarities

It is understood that as discussed herein the use of the terms homology and identity mean the same thing as similarity. Thus, for example, if the use of the word homology is used between two sequences (non-natural sequences, for example) it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed riboswitches, aptamers, expression platforms, genes and proteins herein, is through defining the variants and derivatives in terms of homology to specific known sequences. This identity of particular sequences disclosed herein is also discussed elsewhere herein. In general, variants of riboswitches, aptamers, expression platforms, genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to a stated sequence or a native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### J. Hybridization and Selective Hybridization

The term hybridization typically means a sequence driven interaction between at least two nucleic acid molecules, such as a primer or a probe and a riboswitch or a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting nucleic acid is in for example, 10 or 100 or 1000 fold excess. This type of assay can be performed at under conditions where both the limiting and non-limiting nucleic acids are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of nucleic acid that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions can provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### K. Nucleic Acids

There are a variety of molecules disclosed herein that are nucleic acid based, including, for example, riboswitches, aptamers, and nucleic acids that encode riboswitches and aptamers. The disclosed nucleic acids can be made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if a nucleic acid molecule is introduced into a cell or cell environment through for example exogenous delivery, it is advantageous that the nucleic acid molecule be made up of nucleotide analogs that reduce the degradation of the nucleic acid molecule in the cellular environment.

So long as their relevant function is maintained, riboswitches, aptamers, expression platforms and any other oligonucleotides and nucleic acids can be made up of or include modified nucleotides (nucleotide analogs). Many modified nucleotides are known and can be used in oligonucleotides and nucleic acids. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthine-9-yl (1), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino. 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Base modifications often can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference in its entirety, and specifically for their description of base modifications, their synthesis, their use, and their incorporation into oligonucleotides and nucleic acids.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C1 to C10, alkyl or C2 to C10 alkenyl and alkynyl. 2′ sugar modifications also include but are not limited to —O[(CH₂)n O]m CH₃, —O(CH₂)n OCH₃, —O(CH₂)n NH₂, —O(CH₂)n CH₃, —O(CH₂)n—ONH₂, and —O(CH₂)nON[(CH₂)nCH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications can also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs can also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety, and specifically for their description of modified sugar structures, their synthesis, their use, and their incorporation into nucleotides, oligonucleotides and nucleic acids.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference its entirety, and specifically for their description of modified phosphates, their synthesis, their use, and their incorporation into nucleotides, oligonucleotides and nucleic acids.

It is understood that nucleotide analogs need only contain a single modification, but can also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to (base pair to) complementary nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference its entirety, and specifically for their description of phosphate replacements, their synthesis, their use, and their incorporation into nucleotides, oligonucleotides and nucleic acids.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)).

Oligonucleotides and nucleic acids can be comprised of nucleotides and can be made up of different types of nucleotides or the same type of nucleotides. For example, one or more of the nucleotides in an oligonucleotide can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′—O—-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-O-methyl ribonucleotides. Such oligonucleotides and nucleic acids can be referred to as chimeric oligonucleotides and chimeric nucleic acids.

### L. Solid Supports

Solid supports are solid-state substrates or supports with which molecules (such as trigger molecules) and riboswitches (or other components used in, or produced by, the disclosed methods) can be associated. Riboswitches and other molecules can be associated with solid supports directly or indirectly. For example, analytes (e.g., trigger molecules, test compounds) can be bound to the surface of a solid support or associated with capture agents (e.g., compounds or molecules that bind an analyte) immobilized on solid supports. As another example, riboswitches can be bound to the surface of a solid support or associated with probes immobilized on solid supports. An array is a solid support to which multiple riboswitches, probes or other molecules have been associated in an array, grid, or other organized pattern.

Solid-state substrates for use in solid supports can include any solid material with which components can be associated, directly or indirectly. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

An array can include a plurality of riboswitches, trigger molecules, other molecules, compounds or probes immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

Although useful, it is not required that the solid support be a single unit or structure. A set of riboswitches, trigger molecules, other molecules, compounds andor probes can be distributed over any number of solid supports. For example, at one extreme, each component can be immobilized in a separate reaction tube or container, or on separate beads or microparticles.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

Each of the components (for example, riboswitches, trigger molecules, or other molecules) immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

### M. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for detecting compounds, the kit comprising one or more biosensor riboswitches. The kits also can contain reagents and labels for detecting activation of the riboswitches.

### N. Mixtures

Disclosed are mixtures formed by performing or preparing to perform the disclosed method. For example, disclosed are mixtures comprising riboswitches and trigger molecules.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### O. Systems

Disclosed are systems useful for performing, or aiding in the performance of the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising iosensor riboswitches, a solid support and a signal-reading device.

### P. Data Structures and Computer Control

Disclosed are data structures used in, generated by, or generated from, the disclosed method. Data structures generally are any form of data, information, and/or objects collected, organized, stored, and/or embodied in a composition or medium. Riboswitch structures and activation measurements stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Methods

Disclosed are methods for activating, deactivating or blocking a riboswitch. Such methods can involve, for example, bringing into contact a riboswitch and a compound or trigger molecule that can activate, deactivate or block the riboswitch. Riboswitches function to control gene expression through the binding or removal of a trigger molecule. Compounds can be used to activate, deactivate or block a riboswitch. The trigger molecule for a riboswitch (as well as other activating compounds) can be used to activate a riboswitch. Compounds other than the trigger molecule generally can be used to deactivate or block a riboswitch. Riboswitches can also be deactivated by, for example, removing trigger molecules from the presence of the riboswitch. Thus, the disclosed method of deactivating a riboswitch can involve, for example, removing a trigger molecule (or other activating compound) from the presence or contact with the riboswitch. A riboswitch can be blocked by, for example, binding of an analog of the trigger molecule that does not activate the riboswitch.

Also disclosed are methods for altering expression of an RNA molecule, or of a gene encoding an RNA molecule, where the RNA molecule includes a riboswitch, by bringing a compound into contact with the RNA molecule. Riboswitches function to control gene expression through the binding or removal of a trigger molecule. Thus, subjecting an RNA molecule of interest that includes a riboswitch to conditions that activate, deactivate or block the riboswitch can be used to alter expression of the RNA. Expression can be altered as a result of, for example, termination of transcription or blocking of ribosome binding to the RNA. Binding of a trigger molecule can, depending on the nature of the riboswitch, reduce or prevent expression of the RNA molecule or promote or increase expression of the RNA molecule.

Also disclosed are methods for regulating expression of an RNA molecule, or of a gene encoding an RNA molecule, by operably linking a riboswitch to the RNA molecule. A riboswitch can be operably linked to an RNA molecule in any suitable manner, including, for example, by physically joining the riboswitch to the RNA molecule or by engineering nucleic acid encoding the RNA molecule to include and encode the riboswitch such that the RNA produced from the engineered nucleic acid has the riboswitch operably linked to the RNA molecule. Subjecting a riboswitch operably linked to an RNA molecule of interest to conditions that activate, deactivate or block the riboswitch can be used to alter expression of the RNA.

Also disclosed are methods for regulating expression of a naturally occurring gene or RNA that contains a riboswitch by activating, deactivating or blocking the riboswitch. If the gene is essential for survival of a cell or organism that harbors it, activating, deactivating or blocking the riboswitch can in death, stasis or debilitation of the cell or organism. For example, activating a naturally occurring riboswitch in a naturally occurring gene that is essential to survival of a microorganism can result in death of the microorganism (if activation of the riboswitch turns off or represses expression). This is one basis for the use of the disclosed compounds and methods for antimicrobial and antibiotic effects.

Also disclosed are methods for regulating expression of an isolated, engineered or recombinant gene or RNA that contains a riboswitch by activating, deactivating or blocking the riboswitch. The gene or RNA can be engineered or can be recombinant in any manner. For example, the riboswitch and coding region of the RNA can be heterologous, the riboswitch can be recombinant or chimeric, or both. If the gene encodes a desired expression product, activating or deactivating the riboswitch can be used to induce expression of the gene and thus result in production of the expression product. If the gene encodes an inducer or repressor of gene expression or of another cellular process, activation, deactivation or blocking of the riboswitch can result in induction, repression, or de-repression of other, regulated genes or cellular processes. Many such secondary regulatory effects are known and can be adapted for use with riboswitches. An advantage of riboswitches as the primary control for such regulation is that riboswitch trigger molecules can be small, non-antigenic molecules.

Also disclosed are methods for altering the regulation of a riboswitch by operably linking an aptamer domain to the expression platform domain of the riboswitch (which is a chimeric riboswitch). The aptamer domain can then mediate regulation of the riboswitch through the action of, for example, a trigger molecule for the aptamer domain. Aptamer domains can be operably linked to expression platform domains of riboswitches in any suitable manner, including, for example, by replacing the normal or natural aptamer domain of the riboswitch with the new aptamer domain. Generally, any compound or condition that can activate, deactivate or block the riboswitch from which the aptamer domain is derived can be used to activate, deactivate or block the chimeric riboswitch.

Also disclosed are methods for inactivating a riboswitch by covalently altering the riboswitch (by, for example, crosslinking parts of the riboswitch or coupling a compound to the riboswitch). Inactivation of a riboswitch in this manner can result from, for example, an alteration that prevents the trigger molecule for the riboswitch from binding, that prevents the change in state of the riboswitch upon binding of the trigger molecule, or that prevents the expression platform domain of the riboswitch from affecting expression upon binding of the trigger molecule.

Also disclosed are methods for selecting, designing or deriving new riboswitches and/or new aptamers that recognize new trigger molecules. Such methods can involve production of a set of aptamer variants in a riboswitch, assessing the activation of the variant riboswitches in the presence of a compound of interest, selecting variant riboswitches that were activated (or, for example, the riboswitches that were the most highly or the most selectively activated), and repeating these steps until a variant riboswitch of a desired activity, specificity, combination of activity and specificity, or other combination of properties results. Also disclosed are riboswitches and aptamer domains produced by these methods.

Techniques for in vitro selection and in vitro evolution of functional nucleic acid molecules are known and can be adapted for use with riboswitches and their components. Useful techniques are described by, for example, A. Roth and R. R. Breaker (2003) Selection in vitro of allosteric ribozymes. In: Methods in Molecular Biology Series—Catalytic Nucleic Acid Protocols (Sioud, M., ed.), Humana, Totowa, N.J.; R. R. Breaker (2002) Engineered Allosteric Ribozymes as Biosensor Components. Curr. Opin. Biotechnol. 13:31-39; G. M. Emilsson and R. R. Breaker (2002) Deoxyribozymes: New Activities and New Applications. Cell. Mol. Life Sci. 59:596-607; Y. Li, R. R. Breaker (2001) In vitro Selection of Kinase and Ligase Deoxyribozymes. Methods 23:179-190; G. A. Soukup, R. R. Breaker (2000) Allosteric Ribozymes. In: Ribozymes: Biology and Biotechnology. R. K. Gaur and G. Krupp eds. Eaton Publishing; G. A. Soukup, R. R. Breaker (2000) Allosteric Nucleic Acid Catalysts. Curr. Opin. Struct. Biol. 10:318-325; G. A. Soukup, R. R. Breaker (1999) Nucleic Acid Molecular Switches. Trends Biotechnol. 17:469-476; R. R. Breaker (1999) In vitro Selection of Self-cleaving Ribozymes and Deoxyribozymes. In: Intracellular Ribozyme Applications: Principles and Protocols. L. Couture, J. Rossi eds. Horizon Scientific Press, Norfolk, England; R. R. Breaker (1997) In vitro Selection of Catalytic Polynucleotides. Chem. Rev. 97:371-390; and references cited therein; each of these publications being specifically incorporated herein by reference for their description of in vitro selections and evolution techniques.

Also disclosed are methods for selecting and identifying compounds that can activate, deactivate or block a riboswitch. Activation of a riboswitch refers to the change in state of the riboswitch upon binding of a trigger molecule. A riboswitch can be activated by compounds other than the trigger molecule and in ways other than binding of a trigger molecule. The term trigger molecule is used herein to refer to molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques). Non-natural trigger molecules can be referred to as non-natural trigger molecules.

Deactivation of a riboswitch refers to the change in state of the riboswitch when the trigger molecule is not bound. A riboswitch can be deactivated by binding of compounds other than the trigger molecule and in ways other than removal of the trigger molecule. Blocking of a riboswitch refers to a condition or state of the riboswitch where the presence of the trigger molecule does not activate the riboswitch.

Also disclosed are methods of identifying compounds that activate, deactivate or block a riboswitch. For examples, compounds that activate a riboswitch can be identified by bringing into contact a test compound and a riboswitch and assessing activation of the riboswitch. If the riboswitch is activated, the test compound is identified as a compound that activates the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

Identification of compounds that block a riboswitch can be accomplished in any suitable manner. For example, an assay can be performed for assessing activation or deactivation of a riboswitch in the presence of a compound known to activate or deactivate the riboswitch and in the presence of a test compound. If activation or deactivation is not observed as would be observed in the absence of the test compound, then the test compound is identified as a compound that blocks activation or deactivation of the riboswitch.

Also disclosed are methods of detecting compounds using biosensor riboswitches. The method can include bringing into contact a test sample and a biosensor riboswitch and assessing the activation of the biosensor riboswitch. Activation of the biosensor riboswitch indicates the presence of the trigger molecule for the biosensor riboswitch in the test sample. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a riboswitch that includes a conformation dependent label, the signal from which changes depending on the activation state of the riboswitch. Such a biosensor riboswitch preferably uses an aptamer domain from or derived from a naturally occurring riboswitch.

Biosensor ribsowitches can be used to monitor changing conditions because riboswitch activation is reversible when the concentration of the trigger molecule falls and so the signal can vary as concentration of the trigger molecule varies. The range of concentration of trigger molecules that can be detected can be varied by engineering riboswitches having different dissociation constants for the trigger molecule. This can easily be accomplished by, for example, “degrading” the sensitivity of a riboswitch having high affinity for the trigger molecule. A range of concentrations can be monitored by using multiple biosensor riboswitches of different sensitivities in the same sensor or assay.

Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

Disclosed is a method of detecting a compound of interest, the method comprising bringing into contact a sample and a riboswitch, wherein the riboswitch is activated by the compound of interest, wherein the riboswitch produces a signal when activated by the compound of interest, wherein the riboswitch produces a signal when the sample contains the compound of interest. The riboswitch can change conformation when activated by the compound of interest, wherein the change in conformation produces a signal via a conformation dependent label. The riboswitch can change conformation when activated by the compound of interest, wherein the change in conformation causes a change in expression of an RNA linked to the riboswitch, wherein the change in expression produces a signal. The signal can be produced by a reporter protein expressed from the RNA linked to the riboswitch.

Disclosed is a method comprising (a) testing a compound for inhibition of gene expression of a gene encoding an RNA comprising a riboswitch, wherein the inhibition is via the riboswitch, and (b) inhibiting gene expression by bringing into contact a cell and a compound that inhibited gene expression in step (a), wherein the cell comprises a gene encoding an RNA comprising a riboswitch, wherein the compound inhibits expression of the gene by binding to the riboswitch.

Also disclosed is a method of identifying riboswitches, the method comprising assessing in-line spontaneous cleavage of an RNA molecule in the presence and absence of a compound, wherein the RNA molecule is encoded by a gene regulated by the compound, wherein a change in the pattern of in-line spontaneous cleavage of the RNA molecule indicates a riboswitch.

### A. Identification of Antimicrobial Compounds

Riboswitches are a new class of structured RNAs that have evolved for the purpose of binding small organic molecules. The natural binding pocket of riboswitches can be targeted with metabolite analogs or by compounds that mimic the shape-space of the natural metabolite. Riboswitches are: (1) found in numerous Gram-positive and Gram-negative bacteria including Bacillus anthracis, (2) fundamental regulators of gene expression in these bacteria, (3) present in multiple copies that would be unlikely to evolve simultaneous resistance, and (4) not yet proven to exist in humans. This combination of features make riboswitches attractive targets for new antimicrobial compounds. Further, the small molecule ligands of riboswitches provide useful sites for derivitization to produce drug candidates.

Once a class of riboswitch has been identified and its potential as a drug target assessed. (by, for example, determining how many genes in a target organism are regulated by that class of riboswitch), candidate molecules can be identified. The following provides an illustration of this using the SAM riboswitch (see Example 7 of U.S. Application Publication No. 2005-0053951).

SAM analogs that substitute the reactive methyl and sulfonium ion center with stable sulfur-based linkages (YBD-2 and YBD3) are recognized with adequate affinity (low to mid-nanomolar range) by the riboswitch to serve as a platform for synthesis of additional SAM analogs. In addition, a wider range of linkage analogs (N- and C-based linkages) can be synthesized and tested to provide the optimal platform upon which to make amino acid and nucleoside derivations.

Sulfoxide and sulfone derivatives of SAM can be used to generate analogs. Established synthetic protocols described in Ronald T. Borchardt and Yih Shiong Wu, Potential inhibitor of S-adenosylmethionine-dependent methyltransferase. 1. Modification of the amino acid portion of S-adenosylhomocysteine. J. Med. Chem. 17, 862-868, 1974, can be used, for example. These and other analogs can be synthesized and assayed for binding sequentially or in small groups. Additional SAM analogs can be designed during the progression of compound identification based on the recognition determinants that are established in each round. Simple binding assays can be conducted on B. subtilis and B. anthracis riboswitch RNAs as described elsewhere herein. More advanced assays can also be used.

The most promising SAM analog lead compounds must enter bacterial cells and bind riboswitches while remaining metabolically inert. In addition, useful SAM analogs must be bound tightly by the riboswitch, but must also fail to compete for SAM in the active sites of protein enzymes, or there is a risk of generating an undesirable toxic effect in the patient's cells. As a preliminary assessment of these issues, compounds can be tested for their ability to disrupt B. subtilis growth, but fail to affect E. coli cultures (which use SAM but lack SAM riboswitches). To screen for lead compound candidates, parallel bacterial cultures can be grown as follows:

1. B. subtilis can be cultured in glucose minimal media in the absence of exogenously supplied SAM analogs.

2. B. subtilis can be cultured in glucose minimal media in the presence of exogenously supplied SAM analogs (high doses can be selected, to be followed by repeated experiments designed to test a concentration range of the putative drug compound).

3. E. coli can be cultured in glucose minimal media in the presence of exogenously supplied SAM analogs (high doses will be selected, to be followed by repeated experiments designed to test a concentration range of the putative drug compound).

Fitness of the various cultures can be compared by measurement of cellular doubling times. A range of concentrations for the drug compounds can be tested using cultures grown in microtiter plates and analyzed using a microplate reader from another laboratory. Culture 1 is expected to grow well. Drugs that inhibit culture 2 may or may not inhibit growth of culture 3. Drugs that similarly inhibit both culture 2 and culture 3 upon exposure to a wide range of drug concentrations can reflect general toxicity induced by the exogenous compound (i.e., inhibition of many different cellular processes, in addition or in place of riboswitch inhibition). Successful drug candidates identified in this screen will inhibit E. coli only at very high doses, if at all, and will inhibit B. subtilis at much (>10-fold) lower concentrations.

As derivization points on SAM are identified, efficient identification of lead drug compounds will require larger-scale screening of appropriate SAM analogs or generic chemical libraries. A high-throughput screen can be created by one or two different methods using nucleic acid engineering principles. Adaptation of both fluorescent sensor designs outlined below to formats that are compatible with high-throughput screening assays can be accommodated by using immobilization methods or solution-based methods.

One way to create a reporter is to add a third function to the riboswitch by adding a domain that catalyzes the release of a fluorescent tag upon SAM binding to the riboswitch domain. In the final reporter construct, this catalytic domain can be linked to the yitJ SAM riboswitch through a communication module that relays the ligand binding event by allowing the correct folding of the catalytic domain for generating the fluorescent signal. This can be accomplished as outlined below.

SAM RiboReporter Pool Design: A DNA template for in vitro transcription to RNA was constructed by PCR amplification using the appropriate DNA template and primer sequences. In this construct, stem II of the hammerhead (stem P1 of the SAM aptamer) has been randomized to present more than 250 million possible sequence combinations, wherein some inevitably will permit function of the ribozyme only when the aptamer is occupied by SAM or a related high-affinity analog. Each molecule in the population of constructs is identical in sequence except at the random domain where multiple copies of every possible combination of sequence will be represented in the population.

SAM RiboReporter Selection: The in vitro selection protocol can be a repetitive iteration of the following steps:

1. Transcribe RNA in vitro by standard methods. Include [α-³²P] UTP to incorporate radioactivity throughout the RNA.

2. Purify full length RNA on denaturing PAGE by standard methods.

3. Incubate full length RNA (˜100 pmoles) in negative selection buffer containing sufficient magnesium for catalytic activity (20 mM) but no SAM. Incubate 4 h at room temperature (˜23° C.), with thermocycling or alkaline denaturation as needed to preclude the emergence of selfish molecules.

4. Purify full length RNA on denaturing PAGE and discard RNAs that react in the absence of SAM.

5. Incubate in positive selection buffer containing 20 mM Mg²⁺ and SAM (pH 7.5 at 23° C.). Incubate 20 min at room temperature.

6. Purify cleaved RNA on denaturing PAGE to recover switches that bound SAM and allowed self-cleavage of the RNA.

7. Reverse transcribe RNA to DNA.

8. PCR amplify DNA with primers that reintroduced cleaved portion of RNA.

The concentration of SAM in step 4 can be 100 μM initially and can be reduced as the selection proceeds. The progress of recovering successful communication modules can be assessed by the amount of cleavage observed on the purification gel in step 6. The selection endpoint can be either when the population approaches 100% cleavage in 10 nM SAM (conditions for maximal activity of the parental ribozyme and riboswitch) or when the population approaches a plateau in activity that does not improve over multiple rounds. The end population can then be sequenced. Individual communication module clones can be assayed for generation of a fluorescent signal in the screening construct in the presence of SAM.

A fluorescent signal can also be generated by riboswitch-mediated triggering of a molecular beacon. In this design, riboswitch conformational changes cause a folded molecular beacon tagged with both a fluor and a quencher to unfold and force the fluor away from the quencher by forming a helix with the riboswitch. This mechanism is easy to adapt to existing riboswitches, as this method can take advantage of the ligand-mediated formation of terminator and anti-terminator stems that are involved in transcription control.

To use riboswitches to report ligand binding by binding a molecular beacon, the appropriate construct must be determined empirically. The optimum length and nucleotide composition of the molecular beacon and its binding site on the riboswitch can be tested systematically to result in the highest signal-to-noise ratio. The validity of the assay can be determined by comparing apparent relative binding affinities of different SAM analogs to a molecular beacon-coupled riboswitch (determined by rate of fluorescent signal generation) to the binding constants determined by standard in-line probing.

### EXAMPLES

### A. Example 1

### Glycine-Responsive Riboswitches

A previously unknown riboswitch class was discovered in bacteria that is selectively triggered by glycine. A representative of these glycine-sensing RNAs from Bacillus subtilis operates as a rare genetic on switch for the gcvT operon, which codes for proteins that form the glycine cleavage system. Most glycine riboswitches integrate two ligand-binding domains that function cooperatively to more closely approximate a two-state genetic switch. This advanced form of riboswitch may have evolved to ensure that excess glycine is efficiently used to provide carbon flux through the citric acid cycle and maintain adequate amounts of the amino acid for protein synthesis. Thus, riboswitches perform key regulatory roles and exhibit complex performance characteristics that previously had been observed only with protein factors.

Genetic control by riboswitches located within the noncoding regions of mRNAs is widespread among bacteria (Winkler and Breaker, ChemBioChem 4, 1024 (2003); Vitreschak et al., Trends Genet. 20, 44 (2004); Nudler and Mironov, Trends Biochem. Sci. 29, 11 (2004)). About 2% of the genes in Bacillus subtilis are regulated by these metabolite-binding RNA domains (Mandal et al., Cell 113, 577 (2003)). All riboswitches discovered thus far use a single highly structured aptamer as a sensor for their corresponding target molecules. Selective binding of metabolite by the aptamer causes allosteric modulation of the secondary and tertiary structures of the mRNA 5′-untranslated region (5′-UTR), which changes gene expression by one or more mechanisms that influence transcription termination (Mironov et al., Cell 111, 747 (2002); Winkler et al., Proc. Natl. Acad. Sci. U.S.A. 99, 15908 (2002)), translation initiation (Nahvi et al., Chem. Biol. 9, 1043 (2002); Winkler et al., Nature 419, 952 (2002)), or mRNA processing (Sudarsan et al., RNA 9, 644 (2003); Winkler et al., Nature 428, 281 (2004)).

The existence of riboswitches in modern cells implies that RNA molecules have considerable potential for forming intricate structures that are comparable to protein receptors. Furthermore, riboswitches do not have an obligate need for additional protein factors to carry out their gene control tasks and thus serve as economical genetic switches that sense and respond to changes in metabolite concentrations. However, prior to the riboswitches described herein, higher-ordered functions exhibited by some protein factors had not been observed with natural riboswitches. For example, many protein enzymes, receptors, and gene control factors make use of cooperative binding to provide the cell with a means to rapidly respond to small changes in ligand concentrations (for example, Ptashne and Gann, Genes & Signals (Cold Spring Harbor Press, Cold Spring Harbor, N.Y., 2002); Kurganov, Allosteric Enzymes (Wiley, New York, 1978); Antson et al., Nature 374, 693 (1995)).

Highly conserved RNA motifs in numerous bacterial species that have features similar to known riboswitches were identified (Barrick et al., Proc. Natl. Acad. Sci. U.S.A. 101, 6421 (2004)). One of these motifs, termed gcvT (FIG. 1A), is found in many bacteria, where it typically resides upstream of genes that express protein components of the glycine cleavage system. In B. subtilis, a three-gene operon (gcvT-gcvPA-gcvPB) codes for components of this protein complex, which catalyzes the initial reactions for use of glycine as an energy source (Kikuchi, Mol. Cell Biochem. 1, 169 (1973); Duce et al., Trends Plant Sci. 6, 167 (2001)). This example describes analysis of some properties of glycine-responsive riboswitches.

1. Materials and Methods

i. Chemicals and Oligonucleotides.

Glycine, L-alanine, D-alanine, L-serine, L-threonine, sarcosine, β-alanine, glycine hydroxamate, glycyl-glycine, and glycine-2-³H were purchased from Sigma. Mercaptoacetic acid, glycine methyl ester, glycine tert-butyl ester, glycinamide hydrochloride, and aminomethane sulfonic acid were obtained from Aldrich. Oligonucleotides were synthesized by the HHMI Keck Foundation Biotechnology Resource Center at Yale University and purified by denaturing PAGE. DNA was eluted from the gel by crush-soaking in a buffer containing 10 mM Tris-HCl (pH 7.5 at 23° C.), 200 mM NaCl, and 1 mM EDTA, followed by precipitation with ethanol.

ii. Bioinformatics.

Additional gcvT motifs were identified by creating a covariance model (Eddy and Durbin, Nucleic Acids Res. 22, 2079 (1994)) incorporating the conserved sequence and secondary structures derived from the original phylogeny of gcvT-like RNAs (Barrick, et al., Proc. Natl. Acad. Sci. USA 101, 6421 (2004)). Filtering techniques (Weinberg and Ruzzo, Proceedings of the Eight Annual International Conference on Computational Molecular Biology, ACM Press, pp. 243-251 (2004); Weinberg and Ruzzo, Bioinformatics 20 (Suppl. 1), i334 (2004)) were applied to make the scans of bacterial genomes run rapidly, and new motifs were incorporated into the phylogeny to iteratively generate refined covariance models for subsequent scans.

iii. In-Line Probing Assays.

In-line probing of the VC I-II construct (FIG. 1B), derived from the VC 1422 gene from V. cholerae, was carried out with trace amounts of 5′³²P-labeled RNA using methods that are similar to those described elsewhere in Nahvi et al., Chem. Bio 9, 1043 (2002), and Winkler et al., Nature 419, 952 (2002). RNAs were prepared by transcription from the appropriate DNA template carrying a T7 RNA polymerase promoter, which was generated by PCR from V. cholerae (C6706-st2) genomic DNA using the primers 5′-TAATACGACTCACTATAGGGTTGA-AGACTGCAGGAGAGTGG (SEQ ID NO:8) and 5′-TCCTCTGTCCTTTTGCCTGA SEQ ID NO:9). The underlined nucleotides identify sequences corresponding to the promoter for T7 RNA polymerase.

In a typical in line probing assay, ˜15 nM of labeled RNA is incubated in buffer (20 mM MgCl2, 50 mM Tris, pH 8.3 at 25° C., 100 mM KCl) in the absence or presence of ligand for 40 hrs at 23° C. After incubation, spontaneously cleaved products were separated using 10% denaturing PAGE and were visualized and quantitated using a PhosphorImager (Molecular Dynamics). Nucleotides beyond 210 (FIG. 1A) were not sufficiently resolved to accurately map sites of spontaneous cleavage.

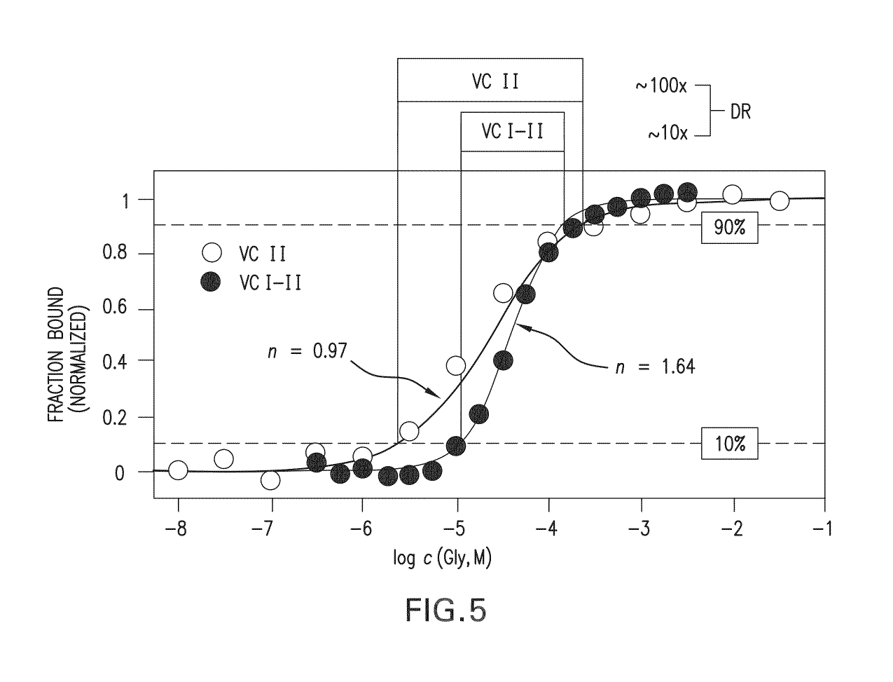

In-line probing of the tandem aptamer construct from B. subtilis (FIG. 8) was carried out using similar methods. PCR DNA template was prepared from B. subtilis genomic DNA (1A2) using the DNA primers 5′-TAATACGACTCACTATAGGGATATGAGCGAATGACAGCAAGGG (SEQ ID NO: 10) and 5′-GGTT CTCTGTCCTGGCACCTGAAAGITTTACTTTGC (SEQ ID NO: 11). Lowercase letters in FIG. 1B and FIG. 8A identify nucleotides that were added to the construct to permit efficient transcription in vitro using RiboMAX transcription (Promega). For the data presented in FIG. 5, fraction bound equals 1 minus the normalized fraction cleaved in the in-line probing assay at U207-C208 for VC II and U74 for VC I-II.

iv. Equilibrium Dialysis Assays.

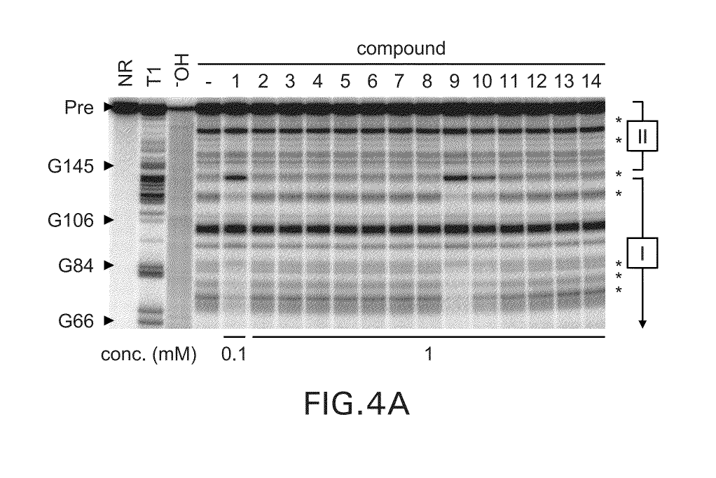

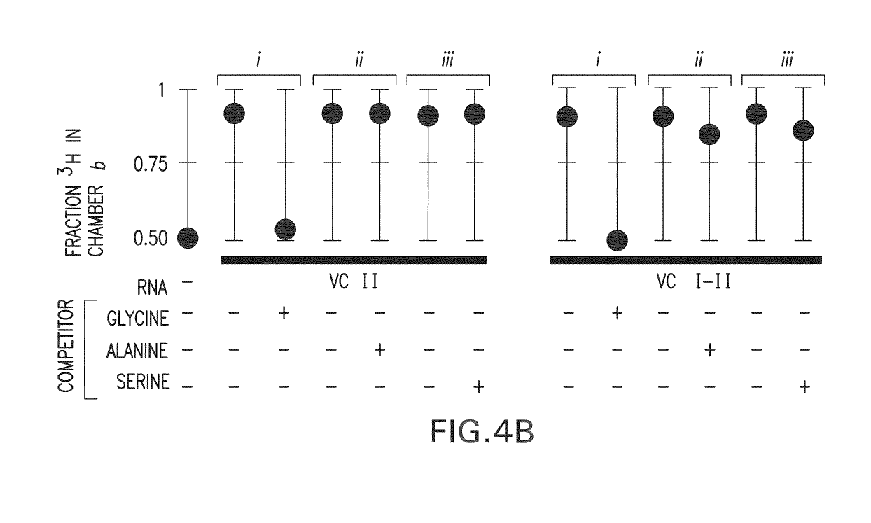

Methods used for equilibrium dialysis were similar to those described in Mandal et al., Cell 113, 577 (2003). Specifically, equilibrium dialysis assays were conducted using a DispoEquilibrium Dialyzer (Harvard Biosciences), wherein chamber a and b are separated by a 5,000 MWCO membrane. Chamber a contained 10 nM of glycine-2-³H in a buffer containing 50 mM Tris-HCl (pH 8.5 at 25° C.), 20 mM MgCl₂ and 100 mM KCl. Chamber b contained VC II or VC I-II RNA transcripts at 100 μM concentration suspended in the same buffer. Equilibrations were allowed to proceed for 10 hrs at 23° C. Subsequently 5 μL of sample was drawn from each chamber and quantitated by liquid scintillation counting. When indicated (FIG. 4B), an excess of 1 mM unlabeled glycine, alanine or serine was delivered to chamber b. For both RNAs, experiments i-iii (FIG. 4B) were conducted by first pre-equilibrating the chambers (left data point), and then adding unlabeled competitor as indicated followed by a second equilibration (right data point). RNAs were prepared by in vitro transcription using the appropriate PCR DNA templates as described above.

v. Single-Round Transcription Assays.

Transcription termination assays were conducted as described previously (Sudarsan et al., Genes Dev. 17, 2688 (2003); Landick et al., Methods Enzymol. 274, 334 (1996)). Transcriptions routinely produced a spurious RNA product band that is labeled “+” in FIG. 8B. This band appears to be caused by spurious transcription initiation at the start of the PCR-generated transcription template, as opposed to initiation at the RNA polymerase promoter sequence. This band is replaced by a slower-migrating product band when additional DNA sequence is present between the promoter sequence and the PCR DNA terminus upstream of the promoter. Analogous spurious transcription products are produced from numerous other PCR-generated transcription templates that are subjected to similar transcription assays, and have not been found to adversely affect function of appropriate-sized riboswitch RNAs.

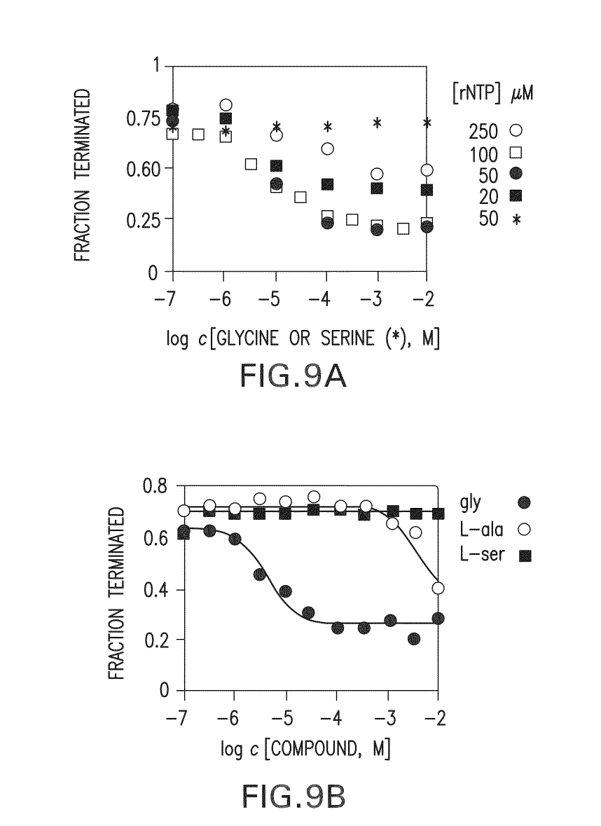

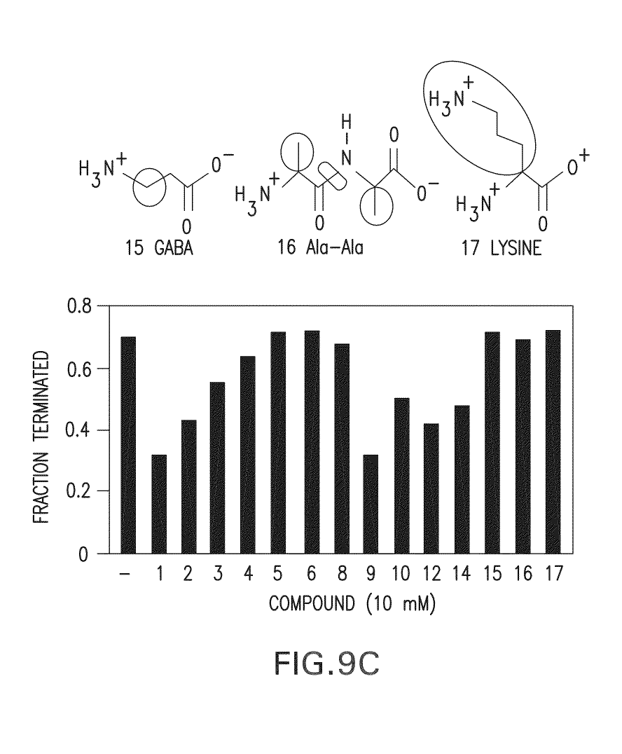

The leakiness of terminator read-through as observed in FIG. 8B can be tuned by adjusting the concentrations of NTPs in the transcription mixture, indicating that conditions in vivo will allow for a more tightly controlled level of production of full-length RNAs (see FIG. 9).

vi. In Vivo Gene Expression Reporter Assays.

A tandem gcvT motif from B. subtilis was fused with a β-galactosidase reporter gene and integrated into the genome of B. subtilis (strain 1A2) using methods described in Mandal et al., Cell 113, 577 (2003); Winkler et al., Nat. Struct. Biol. 10, 701 (2003). Specifically, nucleotides −429 to +7 relative to the B. subtilis gcvT translation start site of the first open reading frame of the gcvT operon was PCR amplified as an EcoR1-BamHI fragment from B. subtilis strain 1A2 (Bacillus Genetic Stock Center, Columbus, Ohio). The wild type construct was cloned into pDG1661 at a site directly upstream of the lacZ reporter gene. The integrity of the constructs were confirmed by sequencing and were used as templates for creating mutants using appropriate primers and Quick Change site-directed mutagenesis kit (Stratagene). The IGR used for this study (FIG. 8A) differed in sequence at three nucleotides (151-153, TTT to AAA) relative to the genomic database. Plasmids generated were integrated into the amyE locus of strain 1A2. Transformants were selected for chloramphenicol (5 μg/ml) resistance and screened for sensitivity to spectinomycin (100 μg/ml). Cells were grown in defined media (0.5% w/v glucose, 2 g/L (NH₄)₂SO₄, 25 g/L K₂HPO₄-3H₂O, 6 g/L KH₂ PO₄, 1 g/L sodium citrate, 0.2 g/L MgSO₄-7H₂O, 2 μM MnCl₂, 15 mM glutamate, and 5 mg/L chloramphenicol) to an A₅₉₅ of 0.1, pelleted, and resuspended in minimal media supplemented with 500 μg/L of amino acid as indicated for each experiment. Cultures were incubated for an additional 3 hr and β-galactosidase assays were performed as described previously (Miller, A Short Course in Bacterial Genetics (Cold Spring Harbor Laboratory Press, Cold Spring Harbor, N.Y., 1992)). Miller units plotted are the average of six values (three assays conducted in duplicate).

2. Results

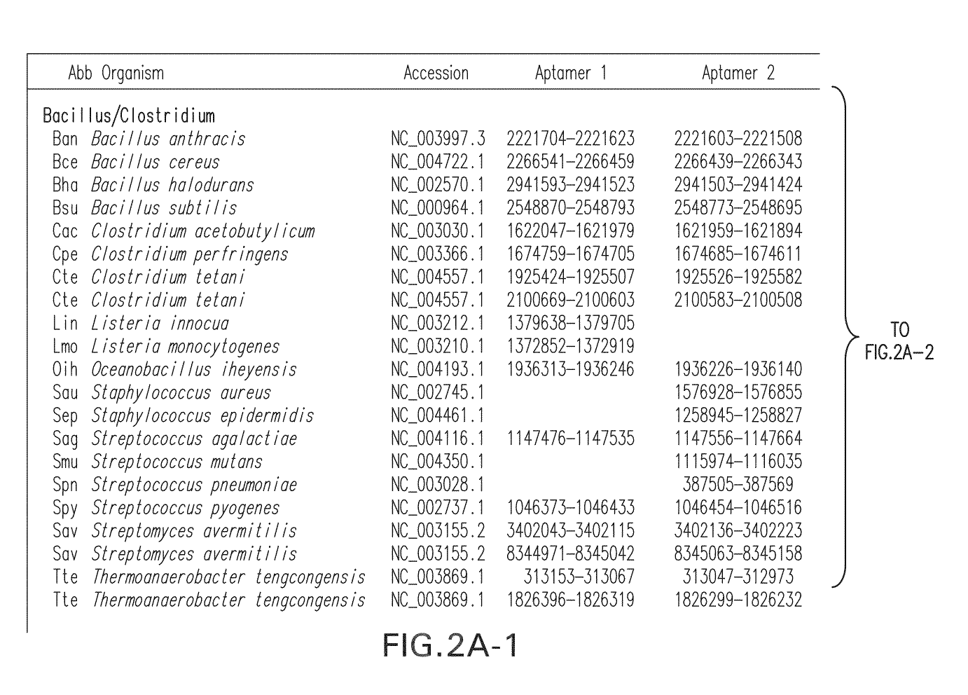

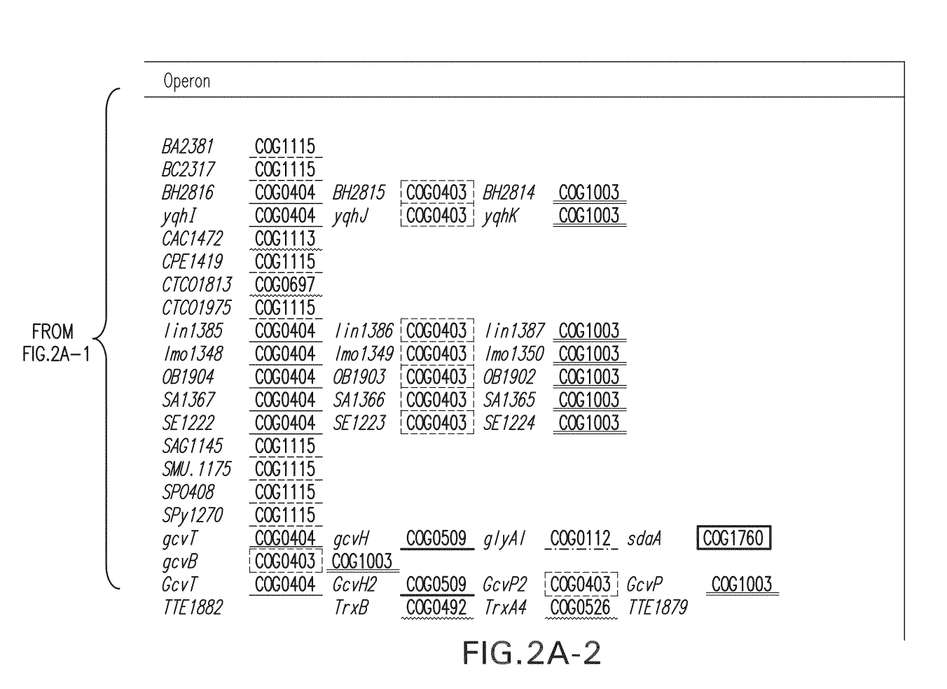

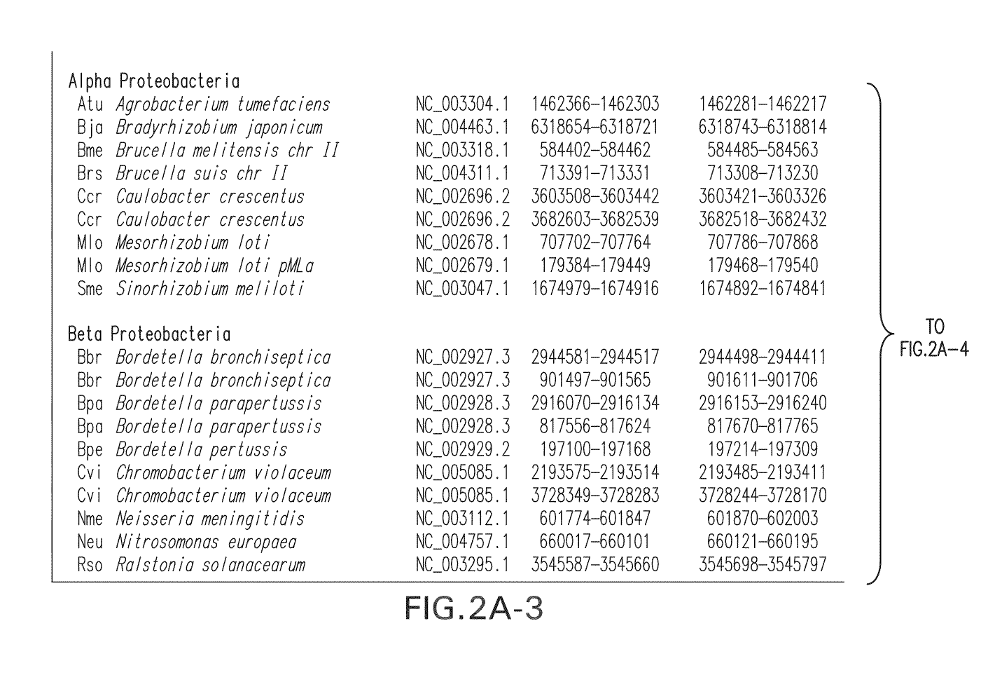

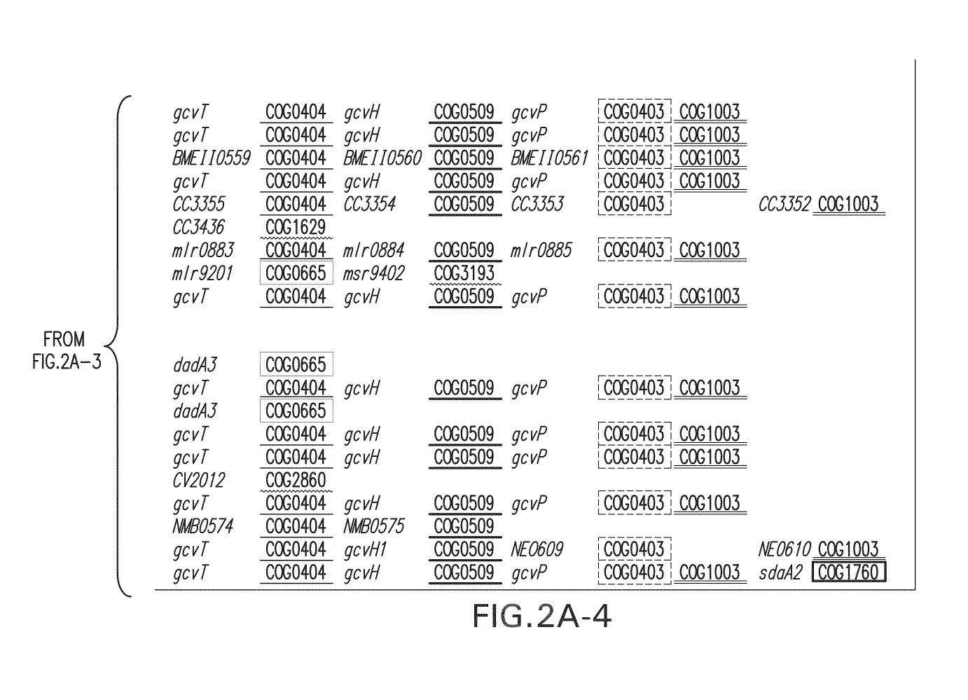

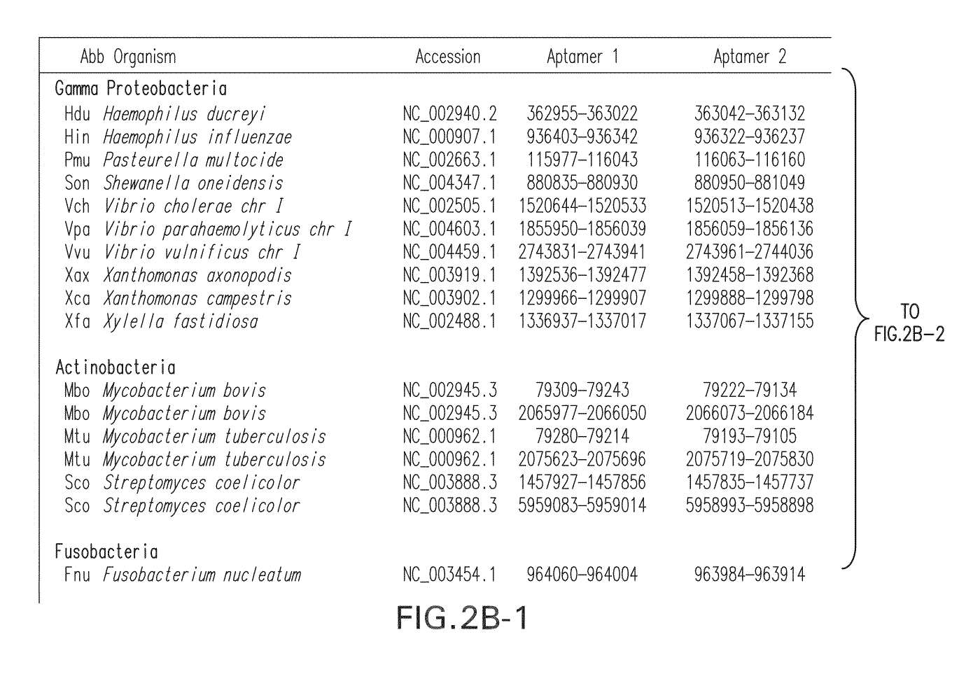

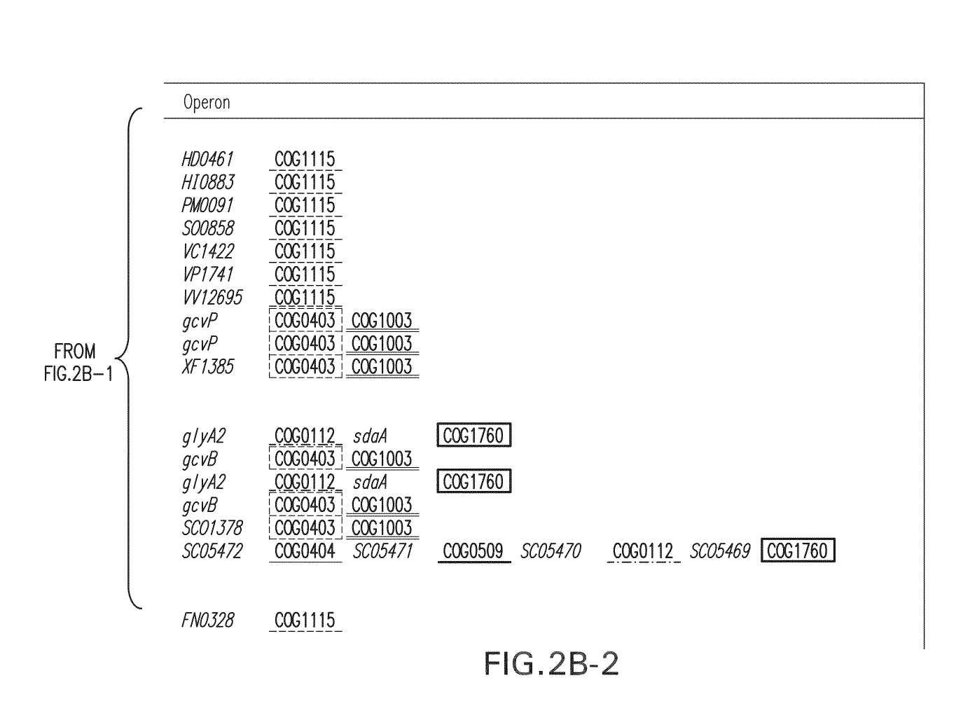

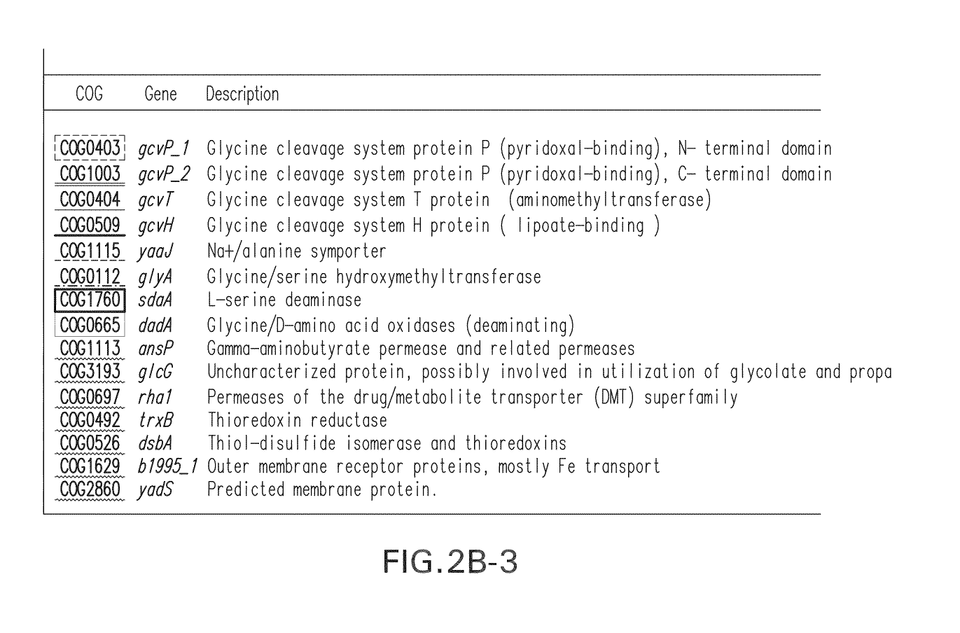

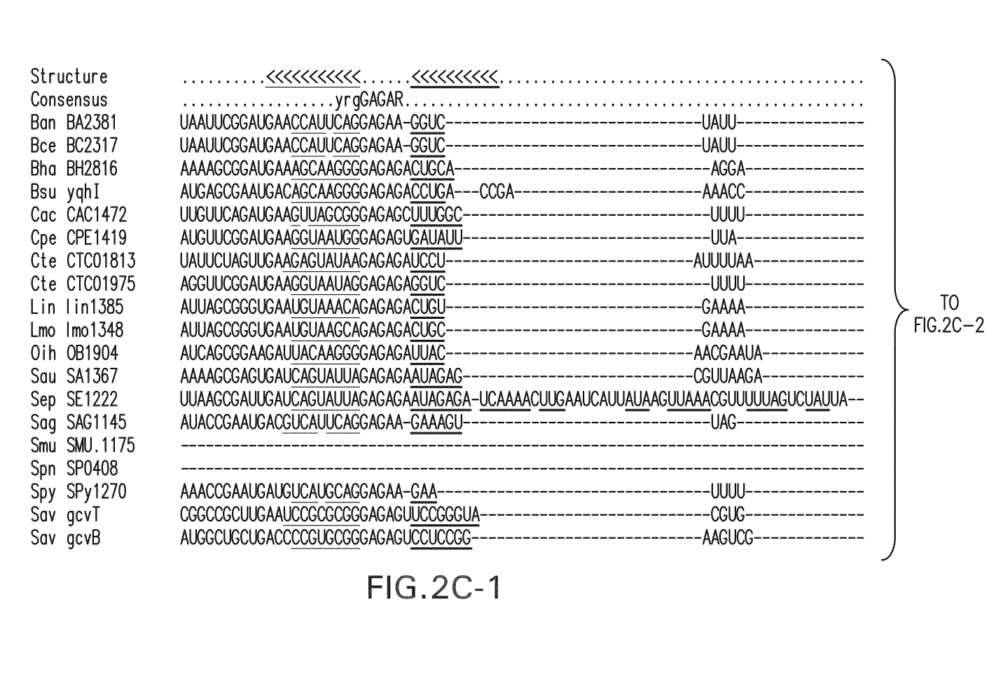

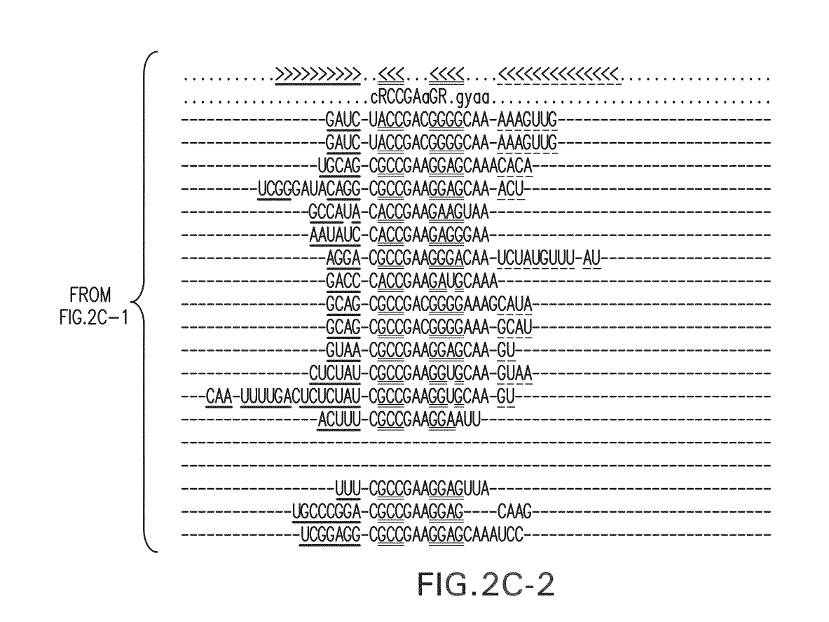

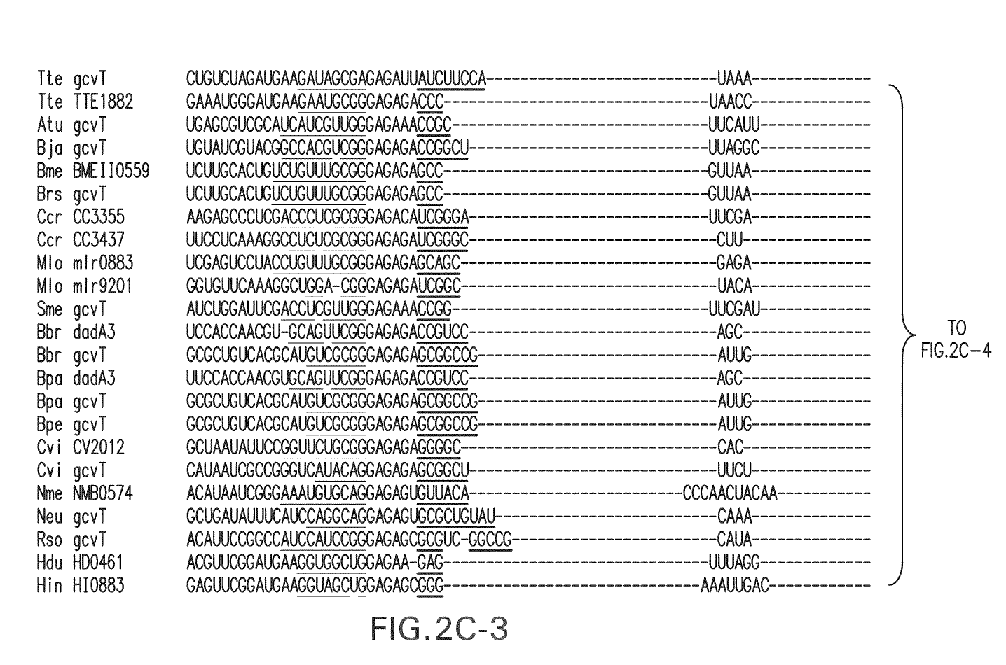

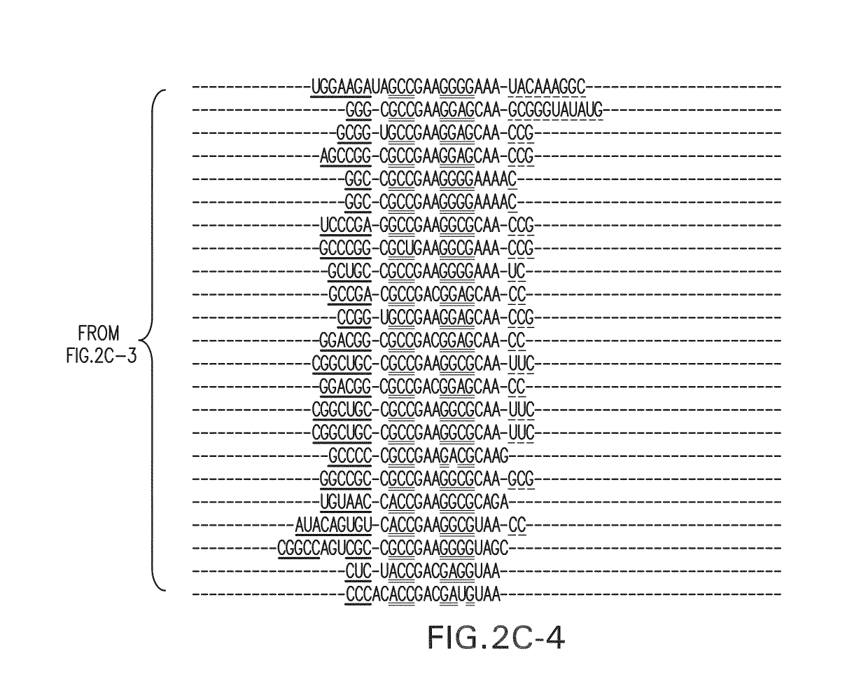

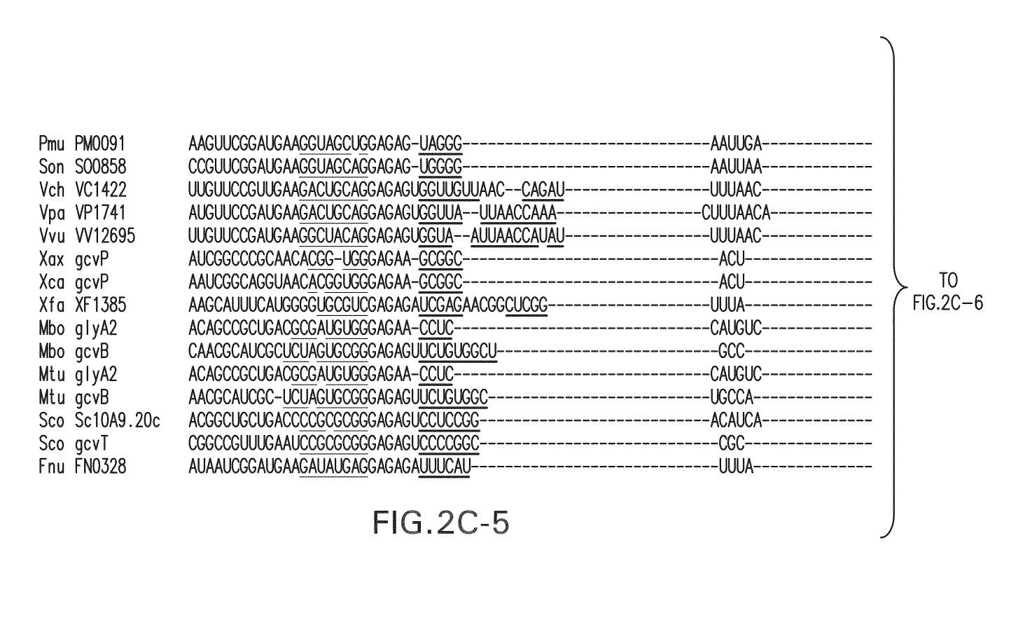

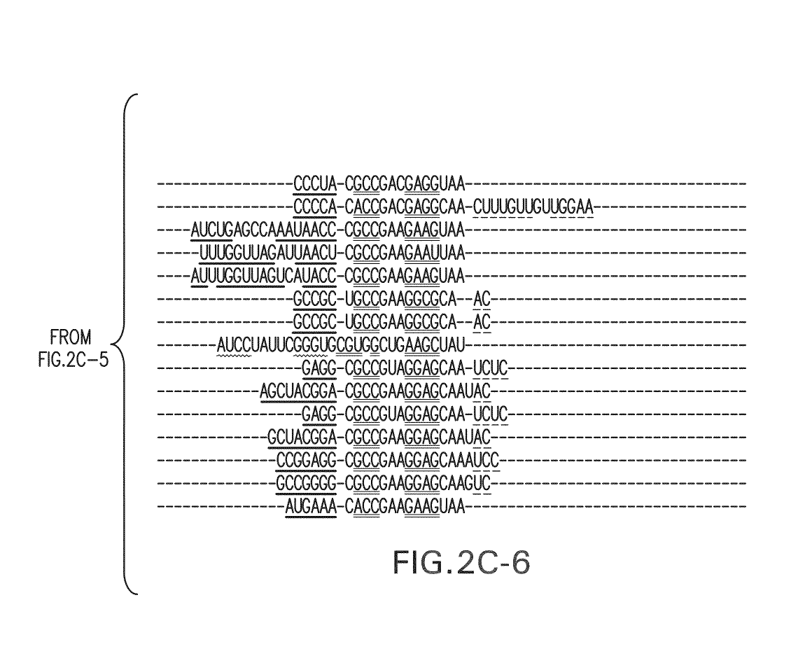

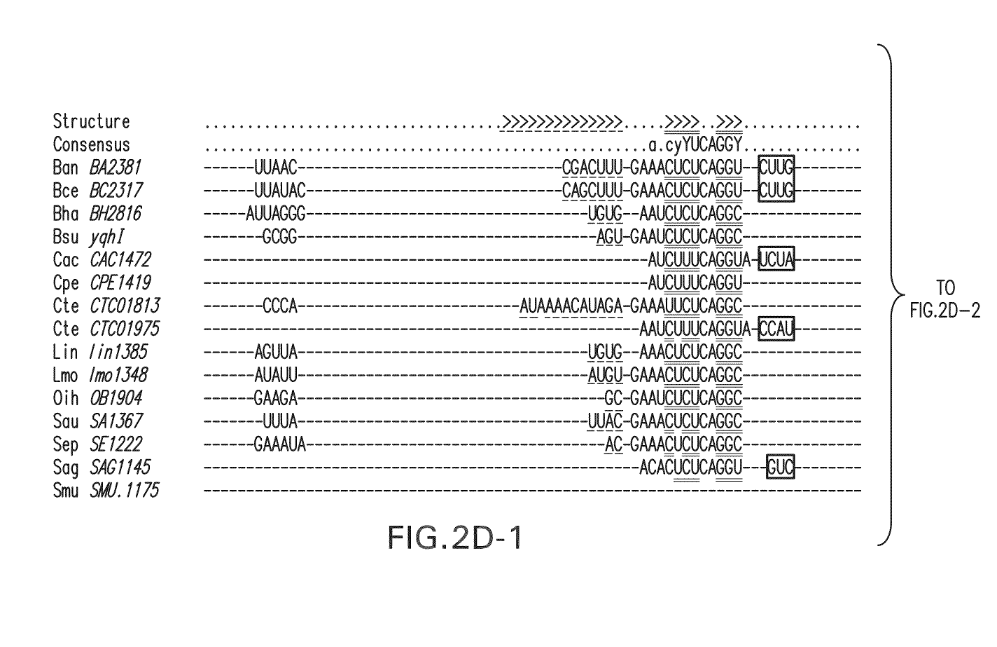

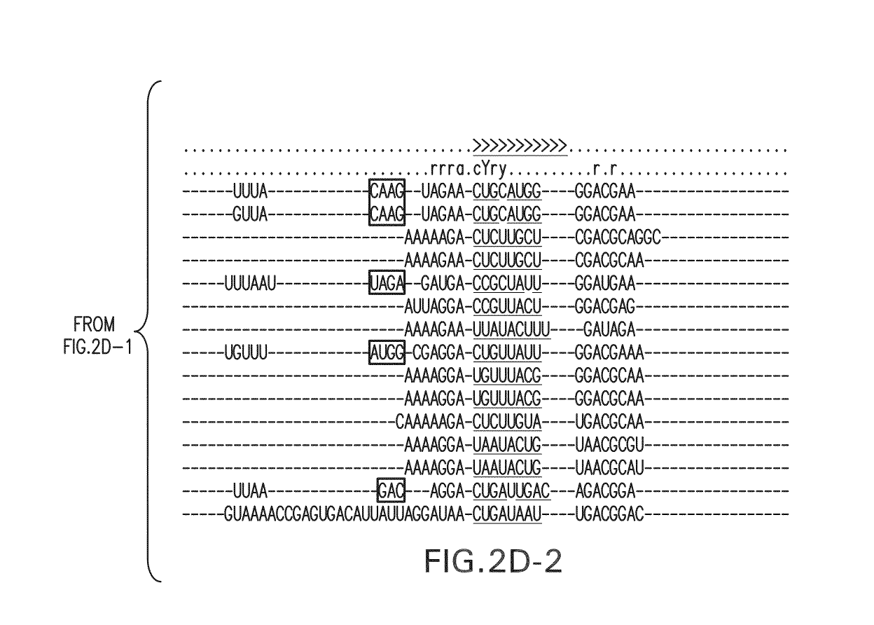

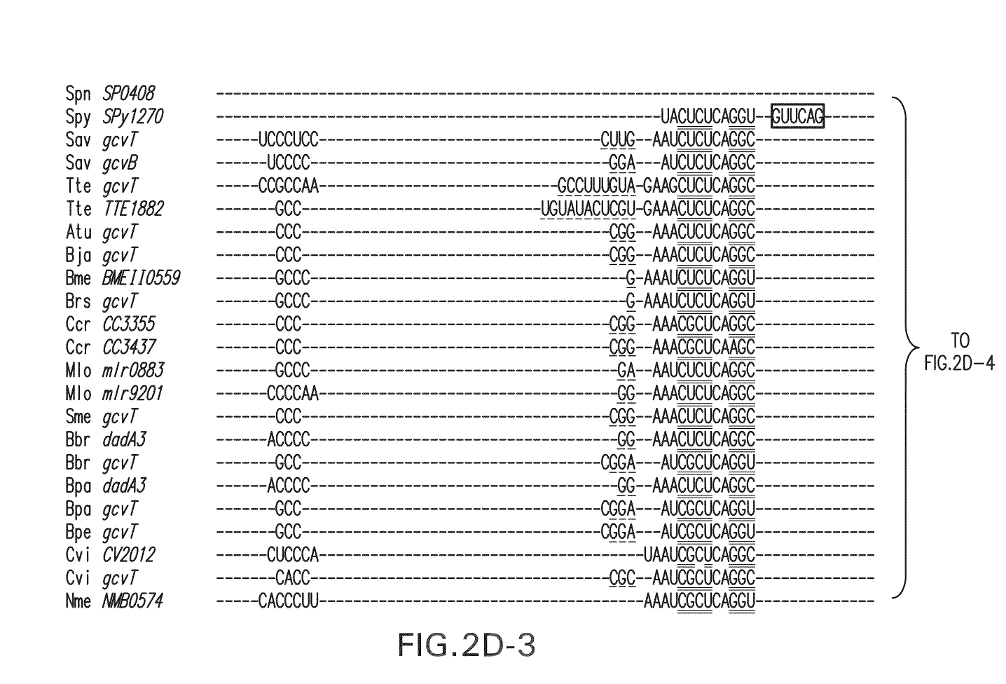

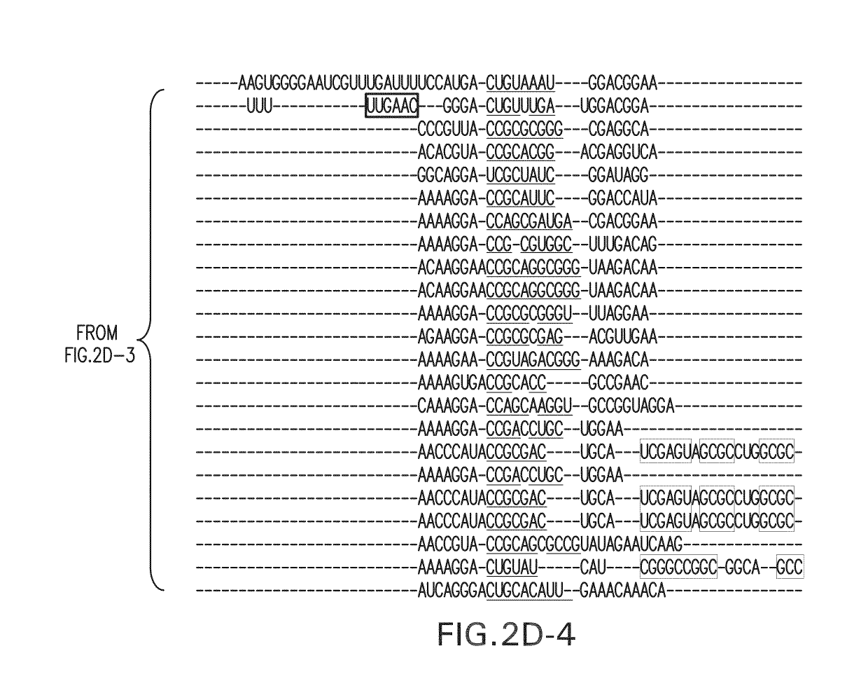

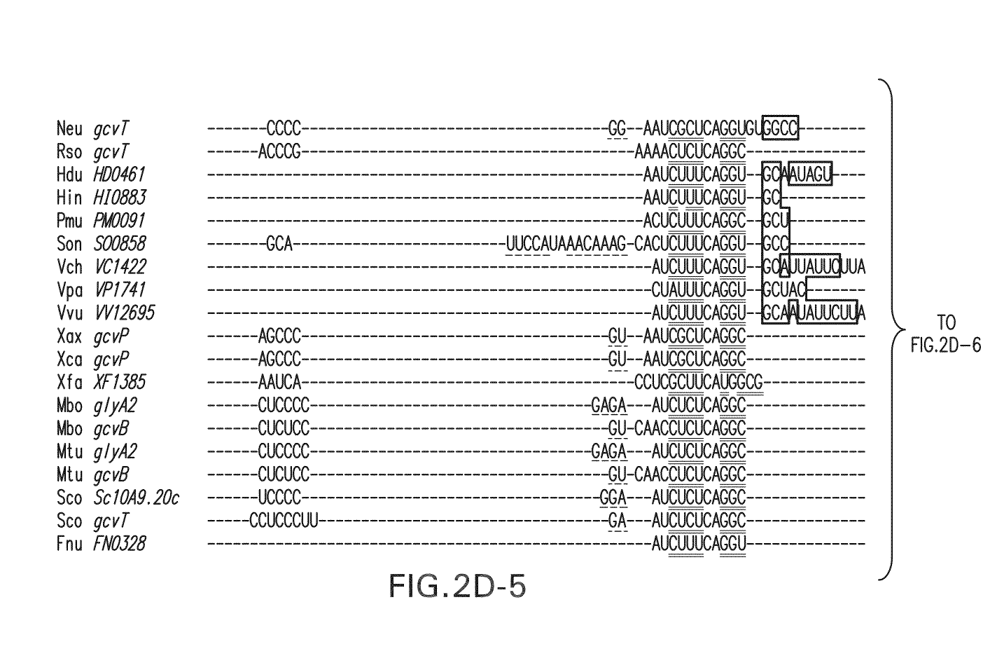

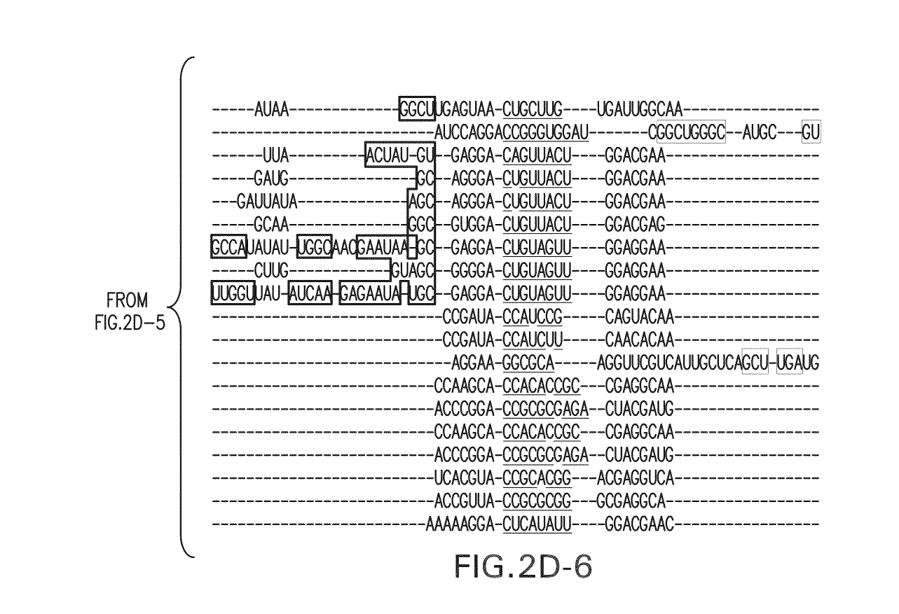

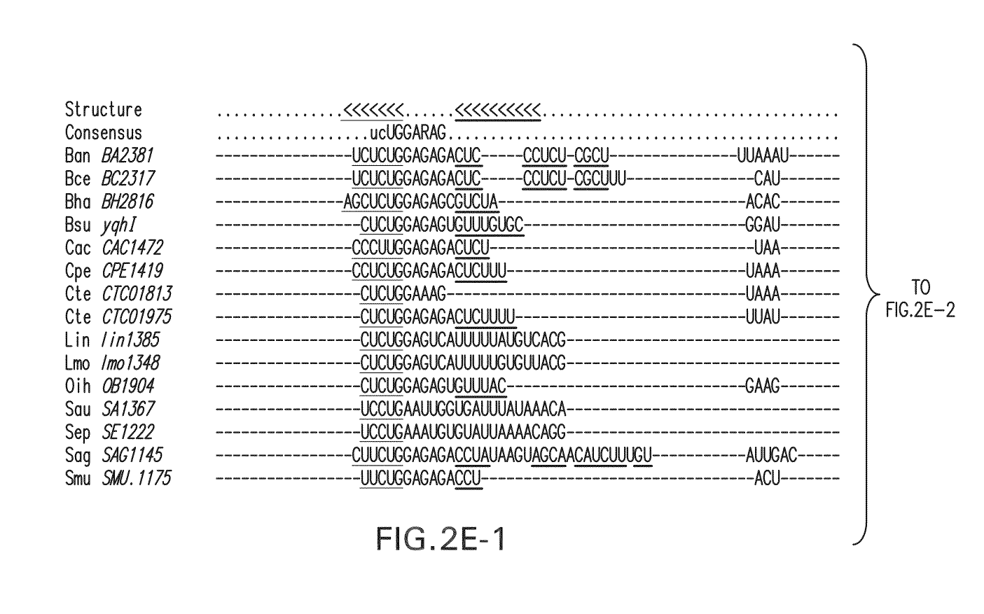

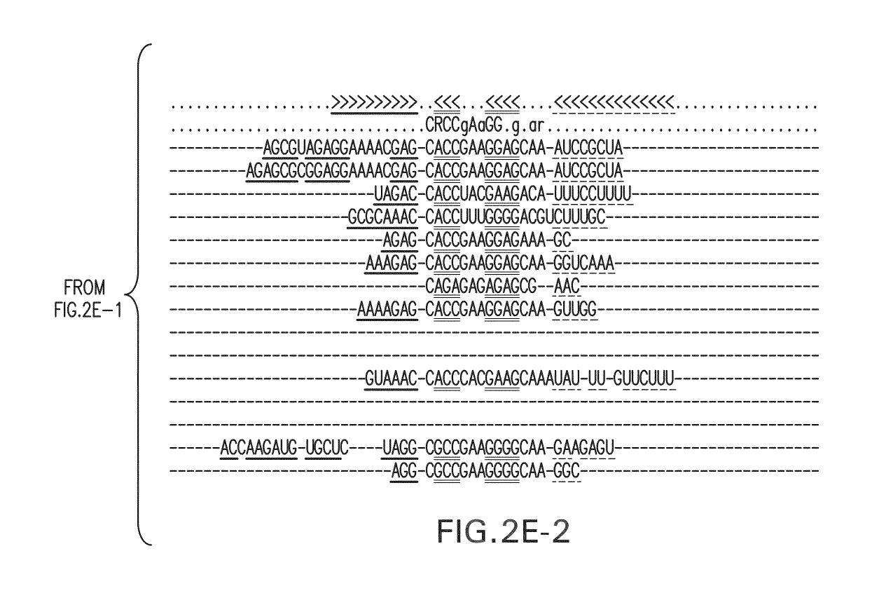

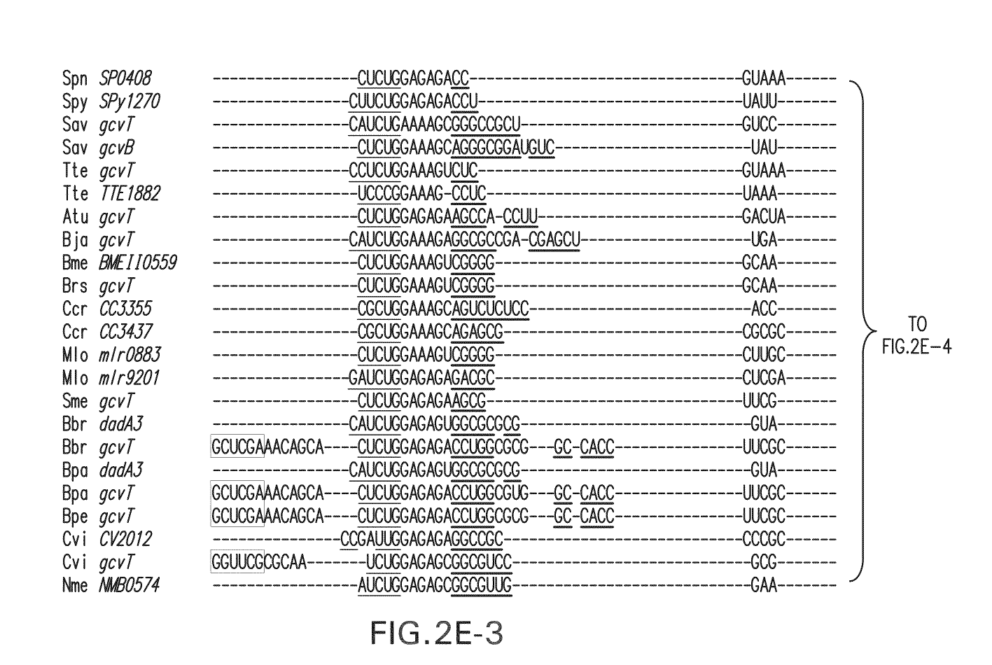

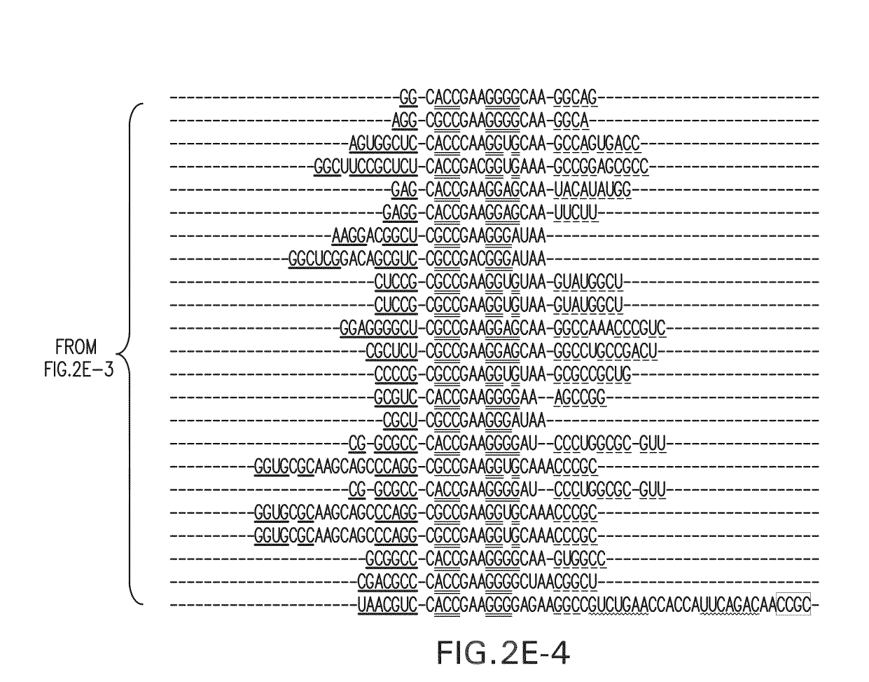

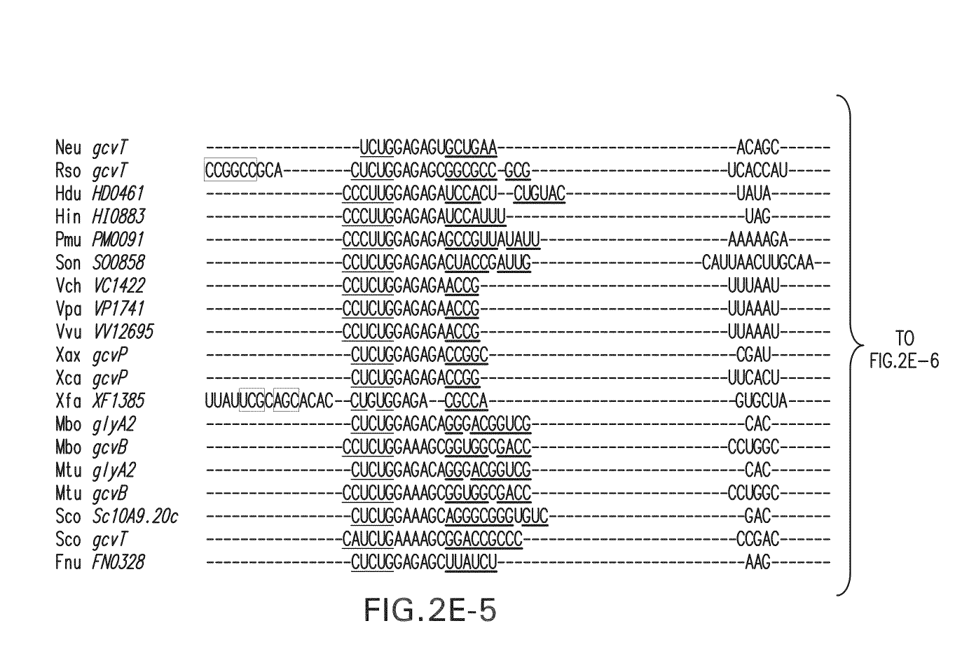

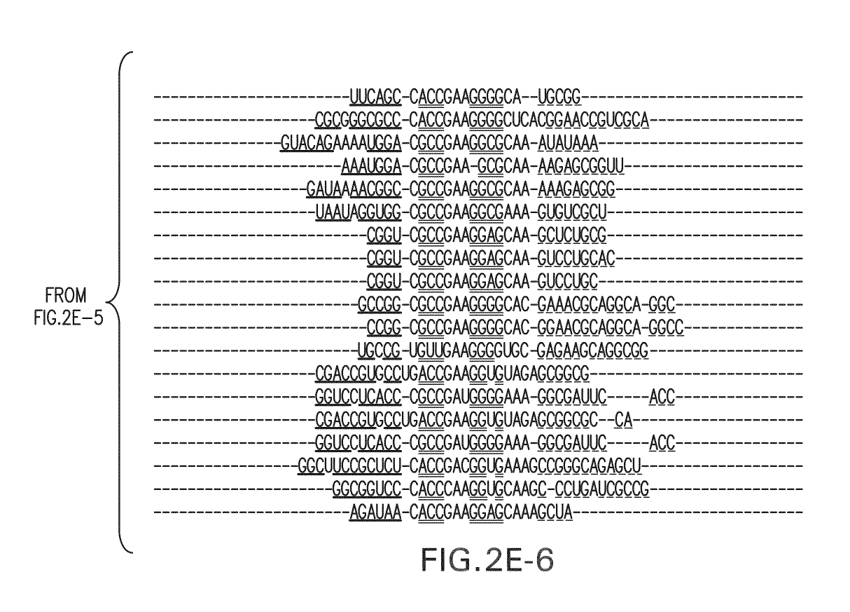

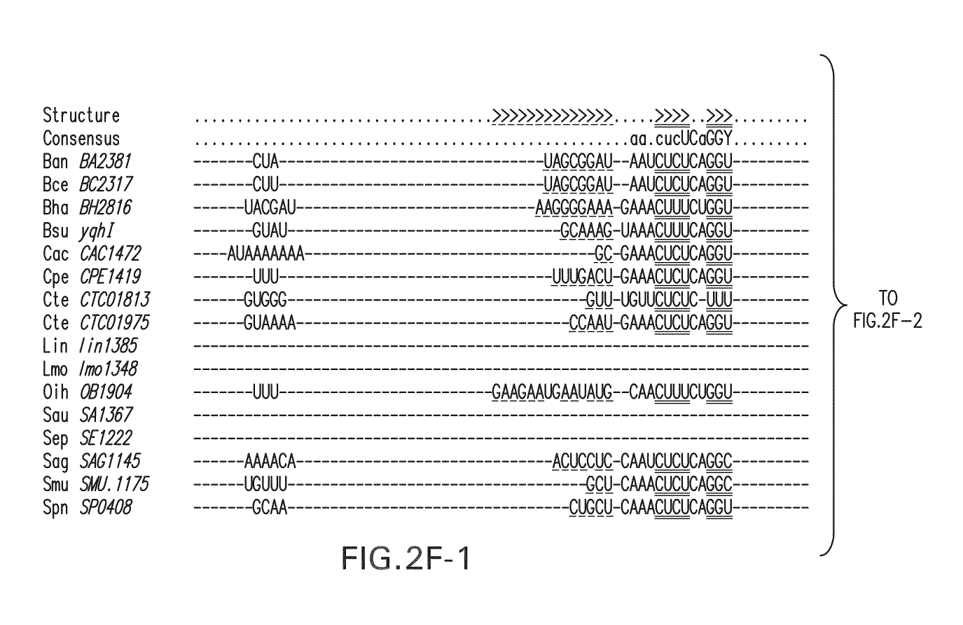

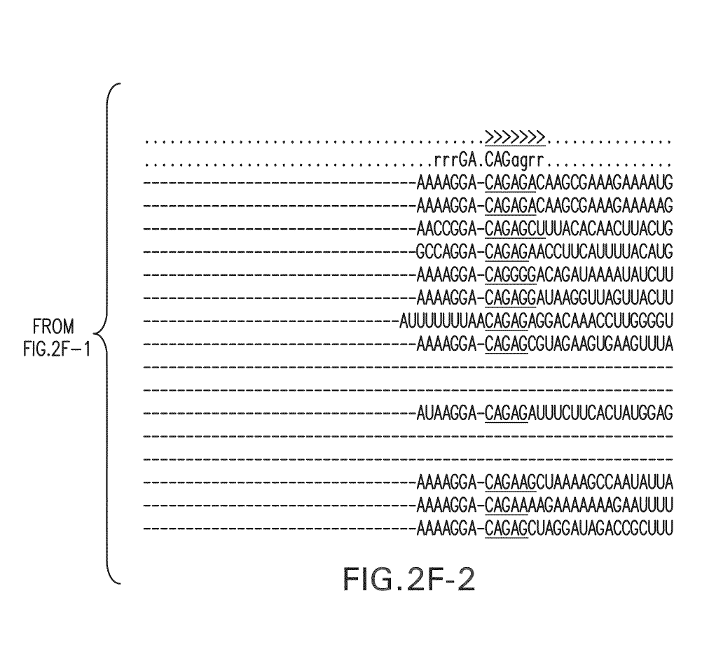

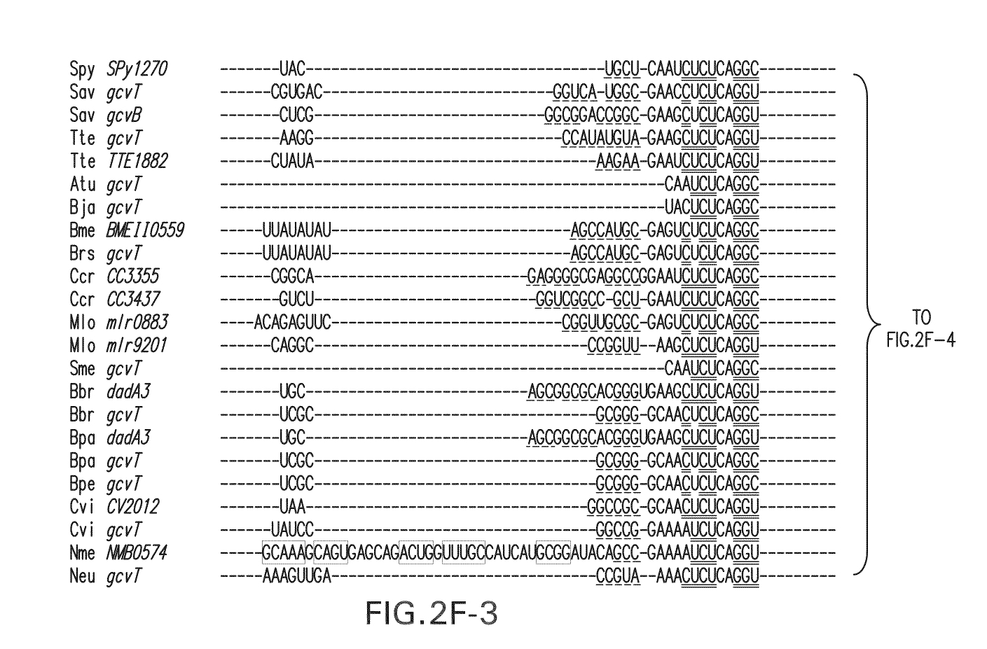

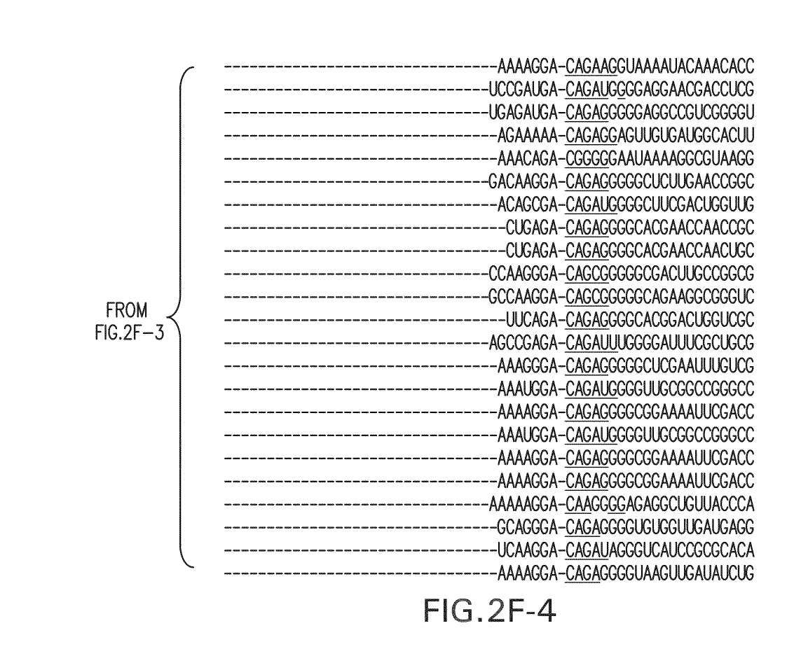

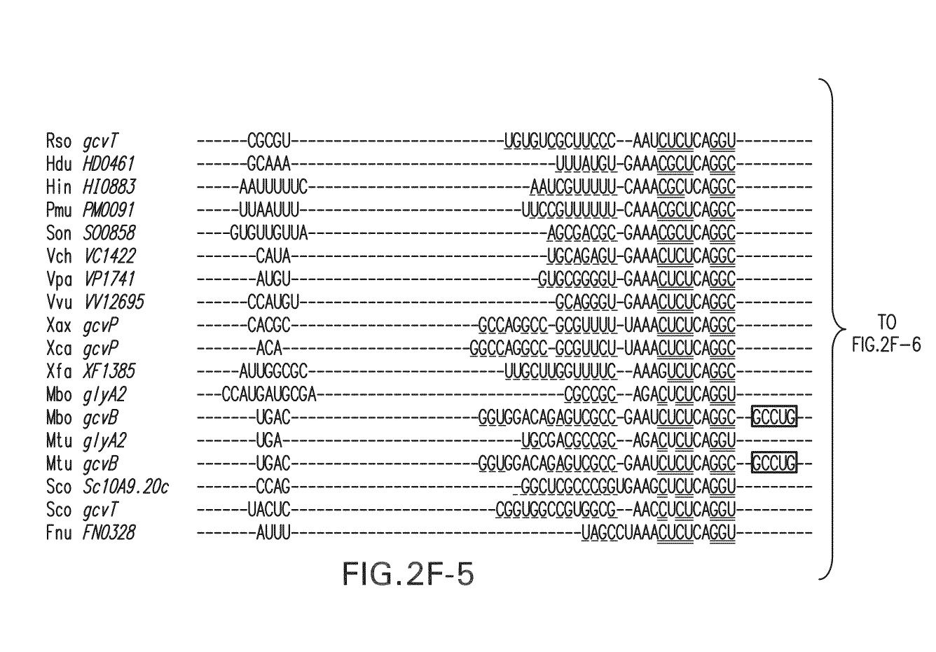

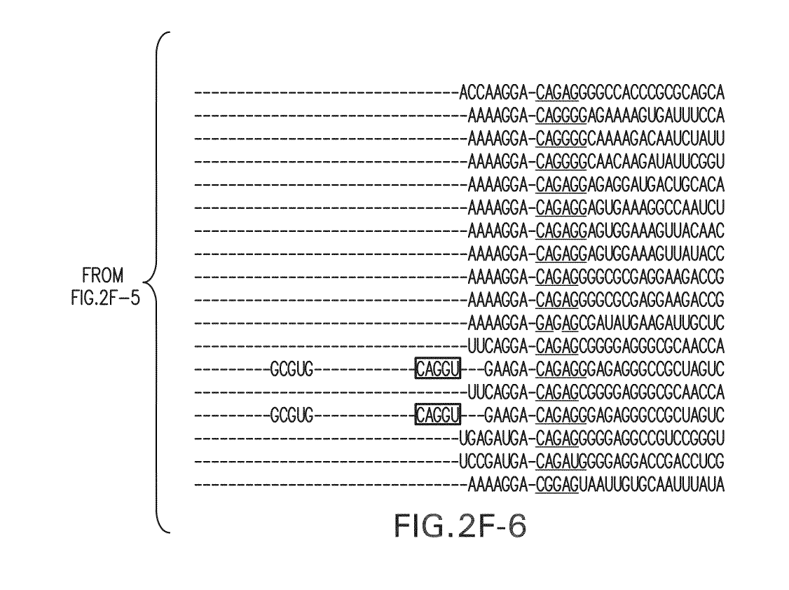

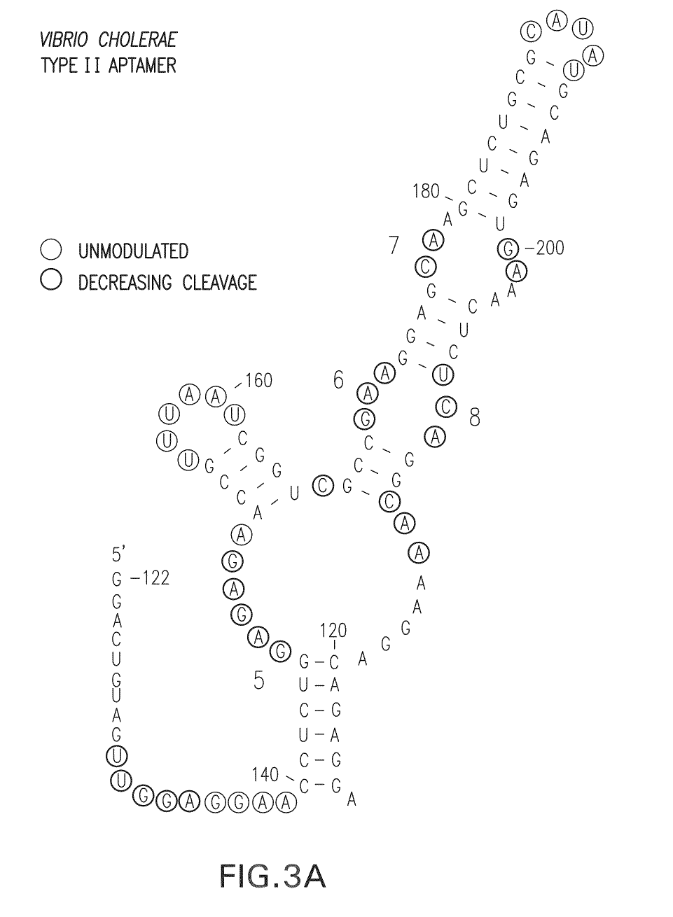

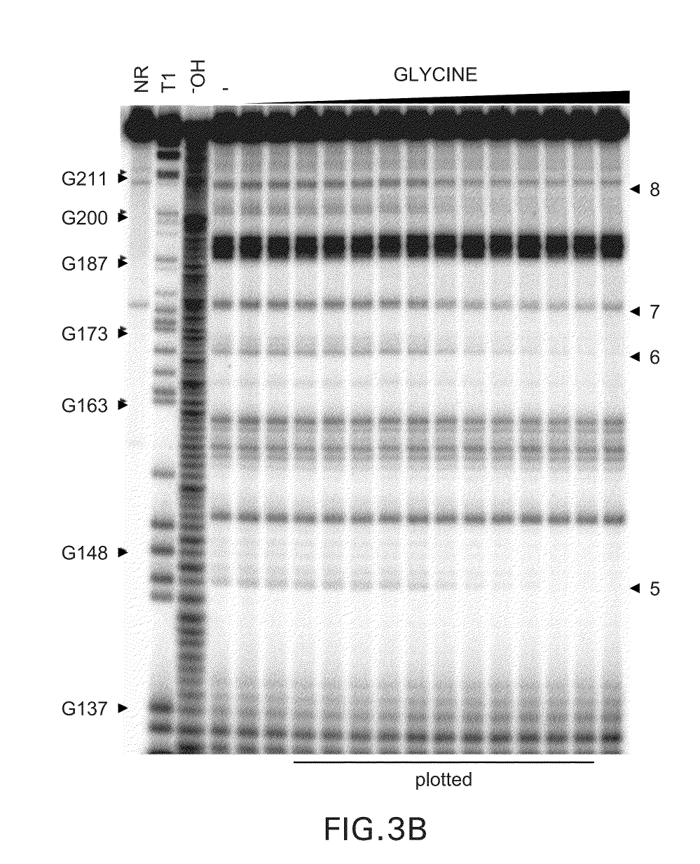

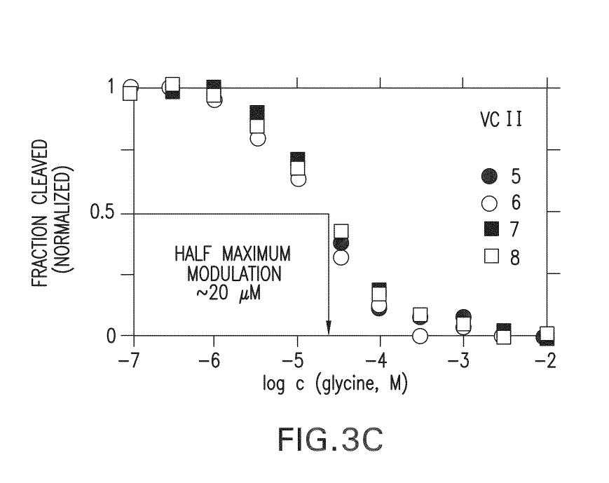

Type I and type II gcvT motifs are natural RNA aptamers for glycine. FIG. 1A shows consensus nucleotides present in more than 80% or 95% of sequences. Representative sequences were identified by bioinformatics (see Materials and Methods; FIG. 2). Circles and thick lines represent nucleotides whose base identities are not conserved. P1 through P4 identify common base-paired elements. ORF refers to open reading frame. FIG. 1B shows patterns of spontaneous cleavage that occur with VC I-II in the absence and presence of glycine are depicted. Numbers adjacent to sites of changing spontaneous cleavage correspond to gel bands denoted with asterisks in FIG. 1C and data sets in FIG. 1D. FIG. 1C shows spontaneous cleavage products of VC I-II upon separation by polyacrylamide gel electrophoresis (PAGE (Nahvi et al., Chem. Biol. 9, 1043 (2002); Winkler et al., Nature 419, 952 (2002)); FIG. 3). NR, T1, and —OH represent no reaction, partial digest with RNase T1, and partial digest with alkali, respectively. Pre refers to precursor RNA. Some fragment bands corresponding to T1 digestion (cleaves after G residues) are labeled. Numbered asterisks identify locations of major structural modulation in response to glycine. The two rightmost lanes carry 1 mM of the amino acids noted. Brackets labeled I and II identify RNA fragments that correspond to cleavage events in the type I and type II aptamers, respectively. FIG. 1D shows plots of the extent of spontaneous cleavage products versus increasing concentrations of glycine for aptamer I (sites 1 through 3), aptamer II (sites 5 through 7), and the linker sequence (site 4). C refers to concentration.

Two forms of the gcvT RNA motif, type I and type II (FIG. 1A), had been identified on the basis of differences in the sequences that flank their conserved cores (Barrick et al., Proc. Natl. Acad. Sci. U.S. 101, 6421 (2004)). More sensitive computational scans (see Materials and Methods) revealed that both motif types reside adjacent to each other, as represented by the architecture of the region immediately upstream of the VC1422 gene (a putative sodium and alanine symporter) from Vibrio cholerae (FIG. 1B). Individually, the type I and type II elements appear to represent separate aptamer domains, wherein each binds a separate target molecule. Furthermore, the linker sequence between the two aptamers exhibits some conservation of both sequence and length, indicating that the aptamers are functionally coupled (FIG. 2).

The metabolite-binding capabilities of V. cholerae RNAs were assessed by using a method termed inline probing (Soukup and Breaker, RNA 5, 1308 (1999)), which can reveal metabolite-induced changes in aptamer structure by monitoring changes in the levels of spontaneous RNA cleavage (Mandal et al., Cell 113, 577 (2003); Winkler et al., Proc. Natl. Acad. Sci. U.S.A. 99, 15908 (2002); Nahvi et al., Chem. Biol. 9, 1043 (2002); Winkler et al., Nature 419, 952 (2002); Sudarsan et al., RNA 9, 644 (2003)). For example, the addition of glycine at 1 mM caused changes in the pattern of spontaneous cleavage of a 226-nucleotide RNA construct (VC I-II) that carries both aptamer types (FIG. 1C), whereas 1 mM L-alanine did not induce change.

Similar results were observed when a 105-nucleotide RNA (VC II) carrying the type II aptamer alone was used for inline probing (FIG. 3). Because both type I and type II domains undergo similar structural changes upon introduction of glycine and because VC II alone exhibits ligand-dependent structural change, each domain serves as a separate glycine binding aptamer. Furthermore, all three sections of the VC I-II construct (aptamer T, linker, and aptamer II) responded to glycine equally at various concentrations (FIG. 1D). This concerted response to glycine indicates that the two aptamers bind glycine in a highly cooperative manner (it is also possible that the two aptamers have perfectly matched affinities for glycine, but cooperative binding is consistent with other properties of the riboswitch).

FIG. 2 shows glycine riboswitch distribution and alignment. FIGS. 2A-2B show Distribution. The indicated positions of each aptamer are for the innermost base pair of the P1 stem in Genbank records. Gene names are from the original sequence files with COGs assigned as previously described (Barrick et al., Proc. Natl. Acad. Sci. U.S.A. 101, 6421 (2004)). FIG. 2C-2F show Alignment. The structure line shows conserved base pairing. Underline style and boxes indicate base pairing in individual aligned sequences. The consensus line shows positions with >95% (uppercase) and >80% (lowercase) sequence conservation (R=A, G; Y═C, U). Representatives that share >90% sequence identity over their entire conserved elements were eliminated before consensus determination.

FIG. 3 shows in-line probing of the VC II RNA construct. FIG. 3A shows sequence, secondary structure, and modulation of the VC II construct. The sites of modulation used for quantitation of glycine-mediated changes in spontaneous cleavage are labeled 5 through 8 as in FIG. 1. FIG. 3B shows PAGE analysis of VC II RNA upon in-line probing with increasing concentrations of glycine. 5′³²P-labeled RNA (˜15 nM) was incubated at 23° C. for 40 hr in 50 mM Tris-HCl (pH 8.3 at 25° C.), 20 mM MgCl₂, and 100 mM KCl in the absence or presence of glycine as indicated. Annotations are as described for FIG. 1C. FIG. 3C shows plot of the normalized fraction of RNA cleaved versus the logarithm of glycine concentration as derived from FIG. 3B. Methods are those as described for FIG. 1.

The molecular recognition specificity of VC I-II was examined by using inline probing with a variety of glycine analogs. The RNA exhibited measurable structural modulation with the methyl ester and tertiary butyl ester analogs of glycine but rejected all other analogs when tested at 1 mM (FIG. 4A). The concentrations of ligand needed to cause half-maximal structure modulation of VC II are about 10 μM for glycine, 100 μM for glycine methyl ester, 1 mM for glycine tertiary butyl ester, and 1 mM for glycine hydroxamate. Specificity for glycine also was observed by using equilibrium dialysis. For example, when an equilibrium dialysis system is preequilibrated with either VC II or VC I-II RNAs, excess glycine restored an equal distribution of 3H-glycine upon subsequent incubation (FIG. 4B). However, the addition of either L-alanine or L-serine failed to restore equal distribution, confirming that the RNAs serve as precise sensors for glycine.

FIG. 4 shows ligand specificity of VC II and VC I-II RNAs. FIG. 4A shows inline probing of VC I-II in the absence (−) or presence of glycine (compound 1) or the analogs L-alanine (2), D-alanine (3), L-serine (4), L-threonine (5), sarcosine (6), mercaptoacetic acid (7), β-alanine (8), glycine methyl ester (9), glycine tert-butyl ester (10), glycine hydroxamate (11), glycinamide (12), aminomethane sulfonic acid (13), and glycyl-glycine (14). Other notations are the same as those described for FIG. 1C. FIG. 4B shows equilibrium dialysis data for VC II and VC I-II (100 μM) in the absence (−) or presence (+) of excess (1 mM) unlabeled glycine, alanine, or serine as indicated. Fraction of 3H-glycine in chamber b reflects the amount of glycine bound by RNA plus half the total amount of free glycine in chambers a and b versus the total amount of 3H-glycine. i to iii represent separate experiments where RNA and 3H are equilibrated (left) and competitor is subsequently added.

The stoichiometry of glycine binding to these RNAs was explored by using equilibrium dialysis with high glycine concentrations. When three equivalents of the amino acid were present versus one equivalent of VC II RNA (100 μM), we observed a shift in glycine distribution that indicates 0.8 equivalents (1 expected) of glycine were bound by RNA. In contrast, when one equivalent of the VC I-II RNA was present (two aptamer equivalents), there is a 1.6-fold increase (2 expected) in the amount of glycine that was bound by RNA. These data provide evidence for a stoichiometry of 1:1 between glycine and each individual aptamer.

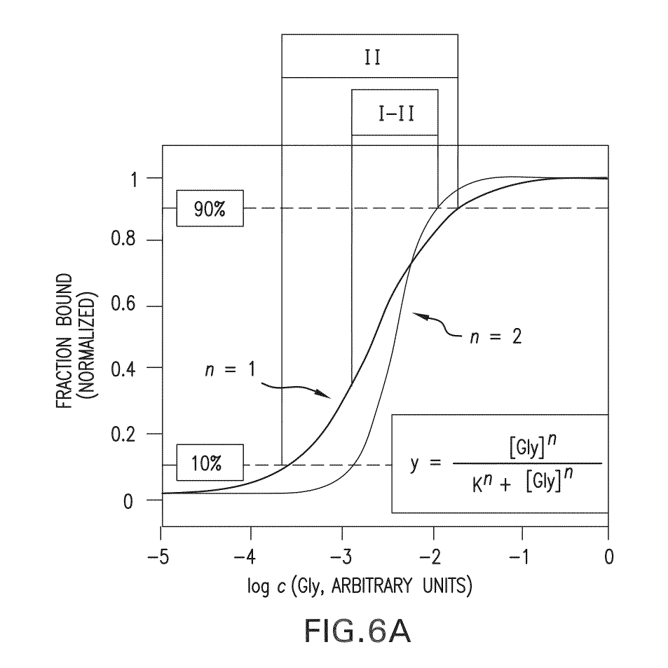

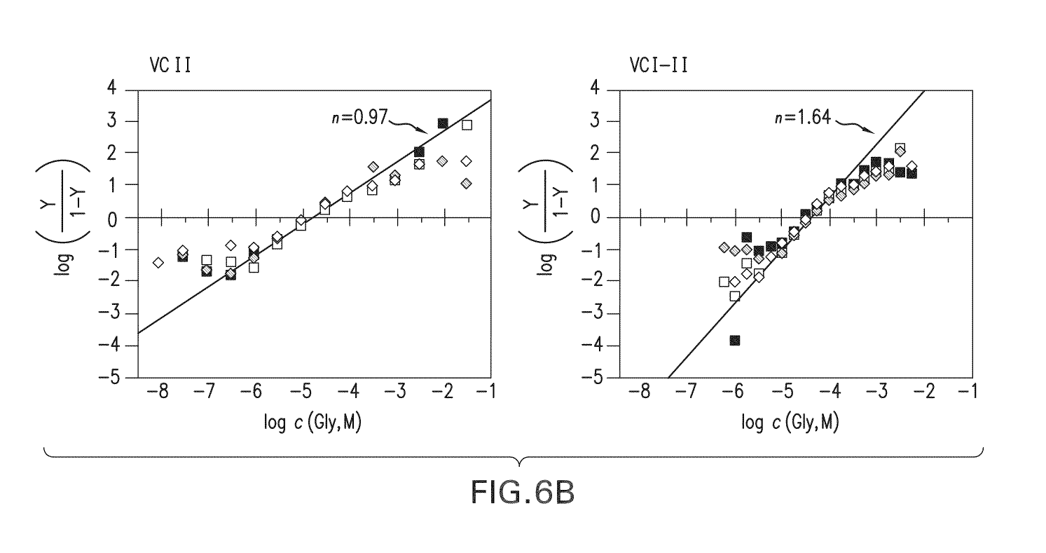

With the two aptamers of VC I-II functioning cooperatively, then structural changes in the RNA will be atypically responsive to increasing glycine concentrations compared with those of a single glycine aptamer. The ligand-dependent modulation of VC II structure by glycine (FIG. 5) was typical of that observed for single aptamer domains of known riboswitches (Mandal et al., Cell 113, 577 (2003); Winkler et al., Proc. Natl. Acad. Sci. U.S.A. 99, 15908 (2002); Nahvi et al., Chem. Biol. 9, 1043 (2002); Winkler et al., Nature 419, 952 (2002); Sudarsan et al., RNA 9, 644 (2003); Winkler et al., Nature Struct. Biol. 10, 701 (2003); Winkler et al., Nature Struct. Biol. 10, 701 (2003); Mandal and Breaker, Nature Struct. Mol. Biol. 11, 29 (2004); Nahvi et al., Nucleic Acids Res. 32, 143 (2004)). The change from 10% to 90% ligand-bound VC I RNA occurred over a 100-fold increase in glycine concentration, which corresponds with the response predicted for a receptor that binds a single ligand (FIG. 6).

In contrast, VC I-II underwent the same change in ligand occupancy over only a 10-fold increase in glycine concentration (FIG. 5). This reduction in the dynamic range for the glycine-mediated response is consistent with glycine binding at one site substantially improving the affinity for glycine binding to the other site. The Hill coefficient (Hill, J. Physiol. 40, iv (1910); Weissbluth, in Molecular Biology Biochemistry and Biophysics, A. Kleinzeller, Ed. (Springer-Verlag, New York, 1974), vol. 15, pp. 27-41) calculated for VC I-II is 1.64, whereas the maximum value for two binding sites is 2. In comparison, the Hill coefficient for the oxygen-carrying protein hemoglobin is 2.8 (Edelstein, Annu. Rev. Biochem. 44, 209 (1975)), whereas the maximum value for four binding sites is 4. Thus, the degree of cooperativity per binding site with the two VC I-II aptamers is equal to or greater than that derived for each of the four sites in hemoglobin.

FIG. 5 shows cooperative binding of two glycine molecules by the VC I-II RNA. Plot depicts the fraction of VC II (open) and VC I-II (solid) bound to ligand versus the concentration of glycine. The constant, n, is the Hill coefficient for the lines as indicated that best fit the aggregate data from four different regions (FIG. 6). Shaded boxes demark the dynamic range (DR) of glycine concentrations needed by the RNAs to progress from 10%- to 90%-bound states.

FIG. 6 shows expected and measured responses to ligand binding with RNA constructs carrying one aptamer or carrying two aptamers that exhibit cooperativity. FIG. 6A shows curves reflecting the empirical equation shown in the inset (Nahvi et al., Nucleic Acids Res. 32, 143 (2004); Hill, J. Physiol. 40, iv (1910)) where the constant, K, is in arbitrary units. Curves for the absence of cooperativity (n=1) and presence of perfect cooperativity at two binding sites (n=2) are depicted. The bars labeled “II” and “I-II” depict the expected range of glycine concentrations needed for the RNA constructs such as VC II and VC I-II to progress from 10% to 90% ligand-bound states. FIG. 6B shows Hill plots for the single glycine riboswitch (VC iH, left panel) or the tandem glycine riboswitch (VC I-II, right panel). The fraction of RNA cleaved at four regions in both RNA constructs was determined by in-line probing. The amount of cleavage was normalized to range from 0 to 1 using the minimum and maximum amounts cleaved for each region. Minimum and maximum amounts cleaved were determined by averaging the 3 lowest and 2 highest glycine concentrations for VC II, and the amount cleaved for the 5 lowest and 4 highest glycine concentrations for the tandem VC I-II RNA (representing the regions where cleavage is essentially constant). For regions that become less ordered upon glycine binding, the fraction of RNA bound to ligand (Y) equals the normalized fraction of RNA cleaved in the in-line probing assay. For regions that become more ordered upon glycine binding, Y equals 1 minus the normalized fraction cleaved. For each group of 4 data sets, the constant, K, and the Hill constant, n, were established to achieve a best fit line to the equation in panel A. For VC II, these values were 24±12 μM and 0.97±0.04, respectively. For VC I-II, these values were 40±1 μM and 1.64±0.07, respectively. The diagonal line in each plot has a slope that reflects these Hill constants. The regions plotted are as follows for VC II: U207-C208, open diamonds; A178, hashed diamonds, G170, open squares, G146, black squares (FIG. 3). The linkages plotted are as follows for VC I-II: G133-G137, open diamonds; A121-G123, hashed diamonds, U74, open squares, U20, black squares (FIG. 1).

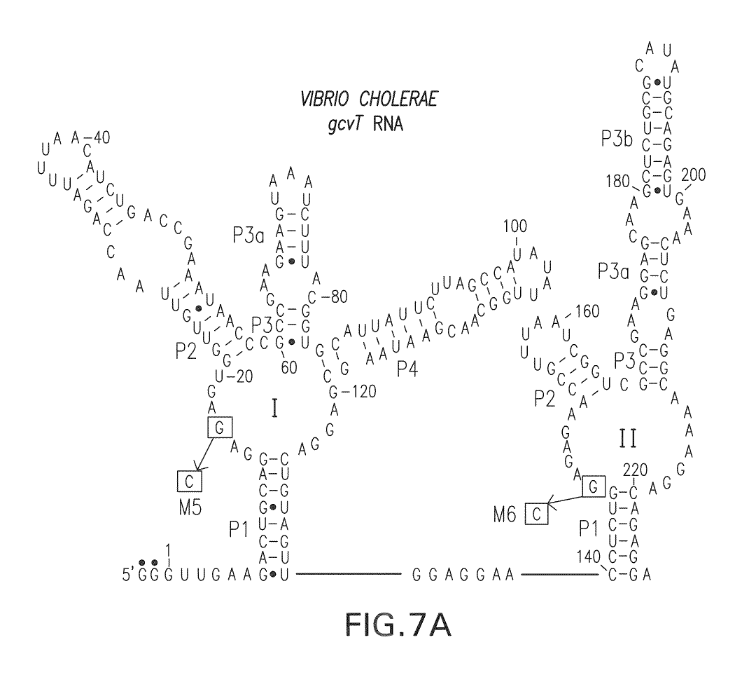

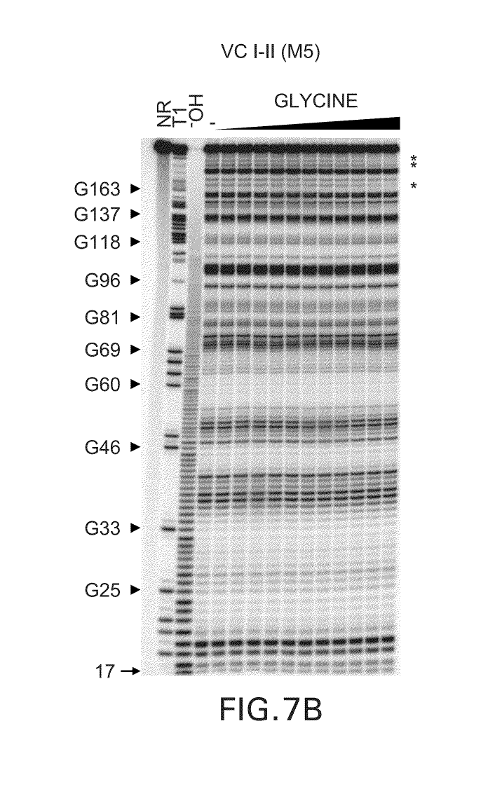

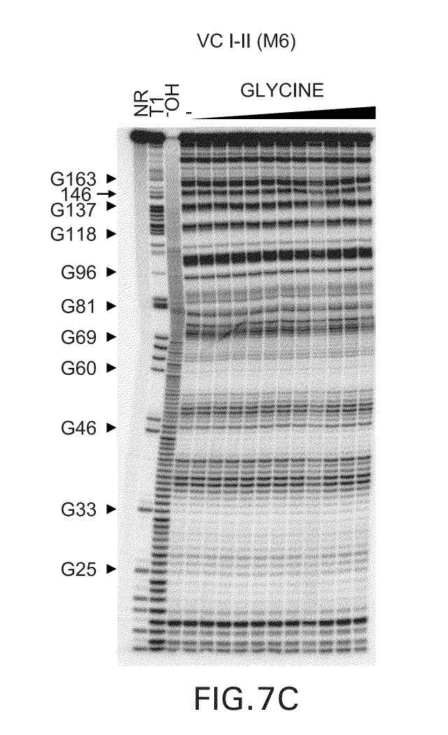

A cooperative mechanism for ligand binding is further supported by the observation that single-point mutations made to either of the conserved cores of VC I-II cause substantial loss of glycine-binding affinity to the mutated aptamer and also cause a dramatic loss of affinity to the unaltered aptamer (FIG. 7). Thus, the binding of glycine at one site induces the adjacent site to exhibit an improvement in ligand binding affinity by 100- to 1000-fold.

FIG. 7 shows evidence for cooperative binding between the type I and type II aptamers of V. cholerae. FIG. 7A shows locations of nucleotide changes that define mutants M5 and M6 for the VC I-II construct. FIG. 7B shows in-line probing of the M5 variant of VC I-II wherein aptamer I has been mutated. Note that G-specific cleavage in the T 1 lane at nucleotide 17 is now absent (arrow). Asterisks identify positions in the unaltered aptamer II domain that modulate upon glycine addition, but at concentrations that are ˜100-fold higher than when aptamer II is in the context of the wild-type VC I-II RNA. Glycine concentrations range from 100 nM to 10 mM. FIG. 7C shows in-line probing of a variant VC I-II construct wherein aptamer II has been mutated. Note that G-specific cleavage in the T1 lane at nucleotide 146 is now absent (arrow). The loss of affinity in the unaltered aptamer I is more than 1,000 fold.

Tandem aptamer architecture (FIG. 8) and selective glycine recognition are also observed with RNA corresponding to the 5′-UTR of the gcvT operon from B. subtilis. This provided a construct that is more amenable to experiments that assess the importance of the gcvT RNA for genetic control. Single-round transcription assays (see Materials and Methods) were used to determine whether a DNA construct corresponding to the intergenic region (IGR) upstream of the B. subtilis gcvT operon yields transcripts whose termination sites are influenced by glycine. In the absence of glycine, only 30% of the RNA products generated by in vitro transcription were full-length (FIG. 8). The remaining 70% were premature termination products that correspond in length to that expected if RNA polymerase stalls at a putative intrinsic terminator (Gusarov and Nudler, Mol. Cell 3, 495 (1999); Yarnell and Roberts, Science 284, 611 (1999)) that partially overlaps the second glycine aptamer (also FIG. 9).

FIG. 8 shows control of B. subtilis gcvT RNA expression in vitro and in vivo. FIG. 8A shows the IGR between the yqhH and gcvT genes of B. subtilis encompassing both aptamers I and II was used for in vitro transcription and in vivo expression assays. Inline probing results were mapped, and mutations used to assess riboswitch function are indicated with boxes. The putative intrinsic terminator stein is labeled “terminator” and is boxed in (bottom right corner). It is expected to exhibit mutually exclusive formation of aptamer II when bound to glycine. nt represents nucleotide. FIG. 8B shows single-round in vitro transcription assays demonstrating that full-length (Full) transcripts are favored when >10 μM glycine is added to the transcription mixture, whereas serine and most glycine analogs (FIG. 9) are rejected by the riboswitch. The line reflects a best-fit curve to an equation reflecting cooperative binding with a Hill coefficient of 1.4. An additional transcription product, termed “+,” appears to be due to spurious transcription initiation (see Materials and Methods). FIG. 8C shows plot of the expression of a 3-galactosidase reporter gene fused to wild-type (WT) gcvT IGR or to a series of mutant IGRs (M1-M6). Data reflect the averages of three assays with two replicates each. Error bars indicate±two standard deviations.

The addition of glycine caused a substantial increase in the amount of full-length RNA transcript relative to the amount of truncated RNA (FIG. 8B). This improvement is induced only by glycine or by other analogs that cause RNA structure modulation. Compounds such as serine, alanine, and other analogs that do not induce modulation also failed to trigger an increase in the production of full-length transcripts (FIG. 9).

Furthermore, the glycine-dependent increase in the yield of full-length transcripts corresponded with that expected for a cooperative RNA switch requiring two ligand binding events. Fitting the transcription data yields a curve that corresponded to cooperative ligand binding, with a Hill coefficient of 1.4 (FIG. 8B). Therefore, transcription control by the gcvT 5′-UTR of B. subtilis responds to glycine with characteristics that parallel those observed when conducting inline probing of the cooperative VC I-II RNA.

To assess whether glycine binding and in vitro transcription control correspond to genetic control events in vivo, reporter constructs were generated by fusing the IGR upstream of the gcvT operon from B. subtilis to a β-galactosidase reporter gene and integrated them into the bacterial genome (see Materials and Methods). The reporter fusion construct carrying the wild-type IGR expresses a high amount of β-galactosidase when glycine is present in the growth medium, whereas a low amount of gene expression results when alanine is present (FIG. 8C). These results indicate that the gcvT motif is part of a glycine-responsive riboswitch with a default state that is off. Glycine binding is required to activate gene expression, as was also observed with the in vitro transcription assays (FIG. 8B).

The importance of several conserved features of the motif were examined by mutating the P1 and P2 stems of the first aptamer domain to disrupt (variants M1 and M3, respectively) and restore (M2 and M4, respectively) base pairing (FIG. 8A). Resulting gene expression levels from constructs carrying the mutant IGRs are consistent with base-paired elements predicted from phylogenetic analyses (Barrick et al., Proc. Natl. Acad. Sci. U.S.A. 101, 6421 (2004)) (FIG. 2). Furthermore, the introduction of mutations into the conserved cores of either aptamer I or aptamer II (variants M5 and M6, respectively) caused a complete loss of reporter gene activation. This latter result indicates that glycine binding to both aptamers is necessary to trigger gene activation, which is consistent with a model wherein cooperative glycine binding is important for riboswitch function.

FIG. 9 shows single-round in vitro transcription of the gcvT 5′-UTR from B. subtilis in the presence of glycine, L-alanine, L-serine, and various glycine analogs. FIG. 9A shows the effect of ribonucleoside triphosphate (rNTP) concentrations on the yields of terminated versus full length RNA transcripts in single-round transcription reactions. Transcription assays were performed using a method adapted from that described earlier (Edelstein, Annu. Rev. Biochem. 44, 209 (1975)). DNA templates were generated by PCR with a primer sequence (5′-CAGCCTATGCAAGAGATT AGAATCTTGATATAATTTATTACAAGATGAATAATATAAGAAAAATCTG; SEQ ID NO: 12) which carries a promoter sequence (underlined) from the xpt-pbuX operon from B. subtilis (Mandal et al., Cell 113, 577 (2003)). DNA templates encompassing nucleotides −406 to +7 relative to the translation start site for the gcvT operon in B. subtilis were used. Transcription assays included 20 mM Tris-HCl (pH 8.0 at 23° C.), 20 mM NaCl, 14 mM MgCl₂, 0.1 mM EDTA, 0.01 mg/mL BSA, and 1% v/v glycerol. Each reaction (10 μL) contained 1 pmole of template DNA and 9 U E. coli RNA polymerase (Epicenter) and was conducted with the type and concentration of target molecule as indicated for each experiment. Transcription was initiated by the addition of the dinucleotide ApA (135 μM), GTP and UTP (2.5 μM each), ATP (1 μM), and [α-³²P]-ATP (4_Ci). After 5 min incubation at 37° C., 50 μM of each NTP was added along with 0.1 mg/mL heparin to prevent re-initiation by RNA polymerase. Transcription products generated after a 10 minute incubation were separated by denaturing 6% PAGE and visualized by using a PhosphorImager. FIG. 9B shows the effects of increasing glycine, L-alanine, and L-serine on transcription termination. Lines depicted for glycine and L-alanine reflect a curve with a Hill coefficient of 1.4, as was determined from the data in FIG. 8B. Single-round transcription assays were conducted as described for FIG. 9A. FIG. 9C shows the specificity of the B. subtilis glycine riboswitch in the presence of 10 mM of test ligands (also see FIG. 4A). Single-round transcription assays were conducted as described for FIG. 9A with 50 μM rNTPs. Analogs of glycine were obtained from Sigma-Aldrich.

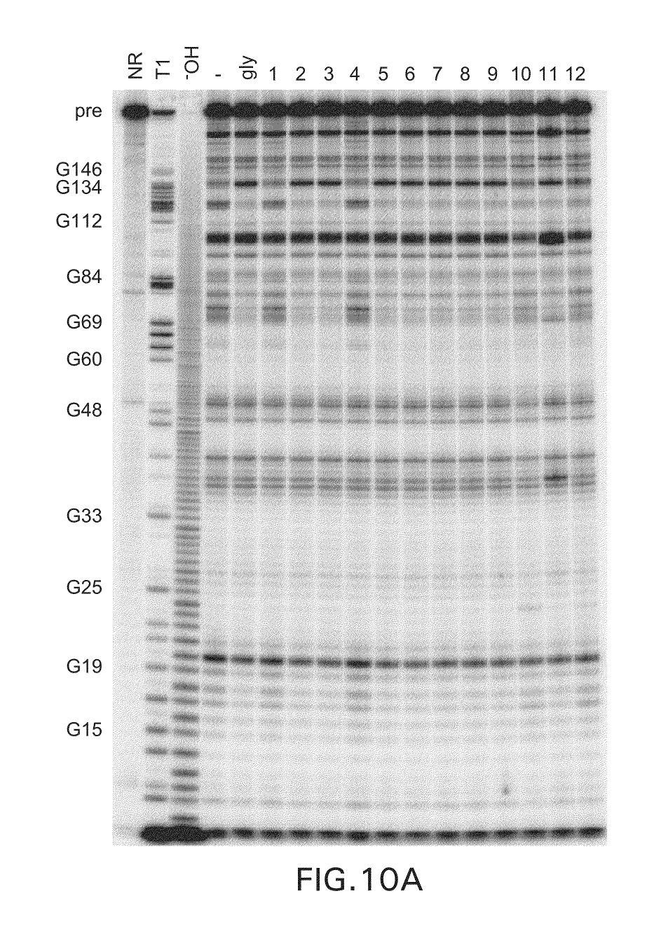

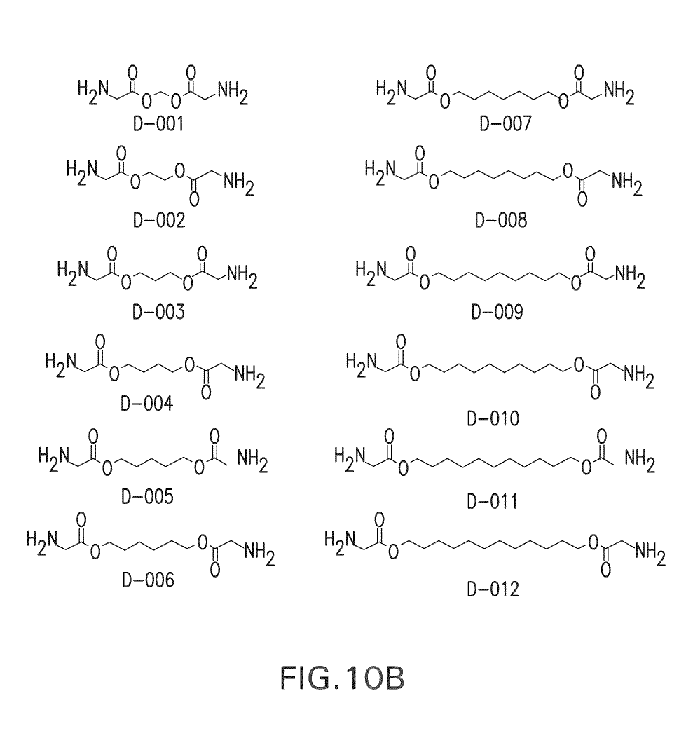

FIGS. 10A and 10B show compounds with conjoined glycine moieties that are bound by a glycine riboswitch. FIG. 10A shows regions of the glycine riboswitch associated with Vibrio cholerae gcvT undergoing structural modulation were determined using in-line probing assays with a 5′³²P-labeled version of the RNA shown. Individual incubations were performed in the absence of ligand (−) or in the presence of 1 mM glycine (gly) and analogs D-001 through D-012 (1-12) as depicted in FIG. 10B. Lanes designated NR, T1, and OH contain RNA that was not reacted, subjected to partial digestion with RNase T1, or subjected to partial alkaline digestion, respectively. Selected RNase T1 cleavage products are identified and correspond to the numbering scheme in FIG. 1B. Pre indicates the position of the full length precursor RNA. Dissociation constants for glycine, D-002 and D-009, derived from separate in-line probing experiments, are approximately 30 μM, approximately 50 μM and approximately 150 μM, respectively.

The glycine-dependent riboswitch is a remarkable genetic control element for several reasons. First, glycine riboswitches form selective binding pockets for a ligand composed of only 10 atoms and thus bind the smallest organic compound among known natural and engineered RNA aptamers. This observation is consistent with the hypothesis that RNA has sufficient structural potential to selectively bind a wide range of biomolecules.

Second, the 5′-UTR of the B. subtilis gcvT operon is a genetic on switch, and thus joins the adenine riboswitch (Mandal and Breaker, Nature Struct. Mol. Biol. 11, 29 (2004)) as a rare type of RNA that has been proven to harness ligand binding and activate gene expression. In most instances, riboswitches cause repression of their associated genes, which is to be expected because many of these genes are involved in biosynthesis or import of the target metabolites. However, the glycine riboswitch from B. subtilis controls the expression of three genes required for glycine degradation. A ligand-activated riboswitch would be required to determine whether sufficient amino acid substrate is present to warrant production of the glycine cleavage system, thereby providing a rationale for why this rare on switch is used.

Third, this is the only known metabolite-binding riboswitch class that regularly makes use of a tandem aptamer configuration. In both V. cholerae and B. subtilis, the juxtaposition of aptamers enables the cooperative binding of two glycine molecules. For the B. subtilis riboswitch, this characteristic results in unusually rapid activation and repression of genes encoding the glycine cleavage system in response to rising and failing concentrations of glycine, respectively. Given the prevalence of the tandem architecture of glycine riboswitches, this more “digital” switch likely gives the bacterium an important selective advantage by controlling gene expression in response to small changes in glycine.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a riboswitch” includes a plurality of such riboswitches, reference to “the riboswitch” is a reference to one or more riboswitches and equivalents thereof known to those skilled in the art, and so forth, “Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

1. A regulatable gene expression construct comprising a nucleic acid molecule encoding an RNA comprising a glycine-responsive riboswitch operably linked to a coding region, wherein the riboswitch regulates expression of the RNA, wherein the riboswitch and coding region are heterologous.

2. The construct of claim 1 wherein the riboswitch comprises an aptamer domain and an expression platform domain, wherein the aptamer domain and the expression platform domain are heterologous.

3. The construct of claim 1 wherein the riboswitch comprises an aptamer domain and an expression platform domain, wherein the aptamer domain comprises a P1 stem, wherein the P1 stem comprises an aptamer strand and a control strand, wherein the expression platform domain comprises a regulated strand, wherein the regulated strand, the control strand, or both have been designed to form a stem structure.

4. The construct of claim 1 wherein the riboswitch comprises two or more aptamer domains and an expression platform domain, wherein at least one of the aptamer domains and the expression platform domain are heterologous.

5. The construct of claim 4 wherein at least two of the aptamer domains exhibit cooperative binding.

6. The construct of claim 1 wherein the riboswitch comprises two or more aptamer domains and an expression platform domain, wherein at least one of the aptamer domains comprises a P1 stem, wherein the P1 stem comprises an aptamer strand and a control strand, wherein the expression platform domain comprises a regulated strand, wherein the regulated strand, the control strand, or both have been designed to form a stem structure.

7. The construct of claim 6 wherein at least two of the aptamer domains exhibit cooperative binding.

8. A riboswitch, wherein the riboswitch is a non-natural derivative of a naturally-occurring glycine-responsive riboswitch.

9. The riboswitch of claim 8 wherein the riboswitch comprises an aptamer domain and an expression platform domain, wherein the aptamer domain and the expression platform domain are heterologous.

10. The riboswitch of claim 9 wherein the riboswitch further comprises one or more additional aptamer domains.

11. The riboswitch of claim 10 wherein at least two of the aptamer domains exhibit cooperative binding.

12. The riboswitch of claim 8 wherein the riboswitch is activated by a trigger molecule, wherein the riboswitch produces a signal when activated by the trigger molecule.

13. A method of detecting a compound of interest, the method comprising bringing into contact a sample and a riboswitch, wherein the riboswitch is activated by the compound of interest, wherein the riboswitch produces a signal when activated by the compound of interest, wherein the riboswitch produces a signal when the sample contains the compound of interest, wherein the riboswitch comprises a glycine-responsive riboswitch or a derivative of a glycine-responsive riboswitch.

14. The method of claim 13 wherein the riboswitch changes conformation when activated by the compound of interest, wherein the change in conformation produces a signal via a conformation dependent label.

15. The method of claim 13 wherein the riboswitch changes conformation when activated by the compound of interest, wherein the change in conformation causes a change in expression of an RNA linked to the riboswitch, wherein the change in expression produces a signal.

16. The method of claim 15 wherein the signal is produced by a reporter protein expressed from the RNA linked to the riboswitch.

17. The construct of claim 13 wherein the riboswitch comprises two or more aptamer domains and an expression platform domain, wherein at least one of the aptamer domains and the expression platform domain are heterologous.

18. The construct of claim 17 wherein at least two of the aptamer domains exhibit cooperative binding.

19. A method comprising
(a) testing a compound for inhibition of gene expression of a gene encoding an RNA comprising a riboswitch, wherein the inhibition is via the riboswitch, wherein the riboswitch comprises a glycine-responsive riboswitch or a derivative of a glycine-responsive riboswitch,
(b) inhibiting gene expression by bringing into contact a cell and a compound that inhibited gene expression in step (a),
wherein the cell comprises a gene encoding an RNA comprising a riboswitch, wherein the compound inhibits expression of the gene by binding to the riboswitch.

20. A method of identifying glycine-responsive riboswitches, the method comprising
assess in-line spontaneous cleavage of an RNA molecule in the presence and absence of glycine, wherein the RNA molecule is encoded by a gene regulated by the compound,
wherein a change in the pattern of in-line spontaneous cleavage of the RNA molecule indicates a riboswitch.

21. (canceled)
